(19) United States
(12) Patent Application Publication
(10) Pub. No.: US 2025/0000993 A1
(43) Pub. Date: Jan. 2, 2025

(54) ANTI-HUMAN CD33 BET DEGRADER ANTIBODY-DRUG CONJUGATES

(71) Applicant: AbbVie Inc., North Chicago, IL (US)

(72) Inventors: Milan Bruncko, Vernon Hills, IL (US); Daniel T. Cohen, Lincolnwood, IL (US); Emily J. Faivre, Lake Forest, IL (US); John E. Harlan, Lake Zurich, IL (US); Matthew M. Ravn, Round Lake Beach, IL (US); Yu Shen, Buffalo Grove, IL (US); George S. Sheppard, Wilmette, IL (US); Le Wang, Vernon Hills, IL (US); Xinxin Zhang, Lake Bluff, IL (US)

(21) Appl. No.: 18/680,477

(22) Filed: May 31, 2024

Related U.S. Application Data
(60) Provisional application No. 63/505,672, filed on Jun. 1, 2023.
(60) Provisional application No. 63/584,449, filed on Sep. 21, 2023.

(51) Int. Cl. A61K 47/68; C07K 16/28

(52) U.S. Cl. CPC A61K 47/6849; A61K 47/6803; A61K 47/6889; C07K 16/2803

(57) ABSTRACT

The present disclosure provides anti-human CD33 bromo- and extra-terminal domain degrader antibody-drug conjugates, including compositions and methods of using such antibody-drug conjugates.

### CROSS REFERENCE TO RELATED APPLICATIONS

This application is a non-provisional application which claims the benefit of, and priority to, U.S. Provisional Application No. 63/505,672, filed on Jun. 1, 2023, as well as U.S. Provisional Application No. 63/584,449, filed on Sep. 21, 2023, the content of which is hereby incorporated by reference in its entirety.

### TECHNICAL FIELD

The present application pertains to anti-human CD33 bromo- and extra-terminal domain degrader antibody-drug conjugates.

### SEQUENCE LISTING

The contents of the electronic sequence listing (ABV21568USO1_ST26.xml; Size: 22,359 bytes; and Date of Creation: May 23, 2024) is herein incorporated by reference in its entirety.

### BACKGROUND

The transcriptional upregulation of oncogenes is a driving force behind the progression of many tumors. Proteins containing bromo- and extra-terminal domain (BET) motifs are key activators of oncogenic networks in a spectrum of cancers. While multiple BET bromodomain inhibitors (BETi) have progressed to clinical development, primarily because of on-target dose-limiting-toxicities such as thrombocytopenia and adverse gastrointestinal effects, promising clinical activities of BETi have only been observed in myeloproliferative neoplasms.

Degraders are bifunctional molecules that employ endogenous protein degradation machinery to degrade targets of interest. BET degradation elicits an inhibition of the core transcriptional elongation complex that is mechanistically distinct from blocking/inhibiting bromodomain interaction with acetylated histones by BETi.

Antibody-drug conjugates (ADCs) represent a class of therapeutics comprising an antibody conjugated to a cytotoxic drug via a chemical linker. The therapeutic concept of ADCs is to combine binding capabilities of an antibody with a drug, where the antibody is used to deliver the drug to a tumor cell by means of binding to a target surface antigen, including target surface antigens that are overexpressed or amplified in the tumor cells.

CD33 is a transmembrane protein that is expressed in acute myeloid leukemia. ADCs that target CD33 have been used to treat AML. Therefore, there is a need for anti-human CD33 BETd ADCs and, in particular, there is a need for anti-human CD33 BETd ADCs that can be used for therapeutic purposes, such as in the treatment of acute myeloid leukemia.

### SUMMARY

The present disclosure provides ADCs that specifically bind to human CD33.

A first aspect comprises anti-human CD33 BETd ADC comprising the following structure:

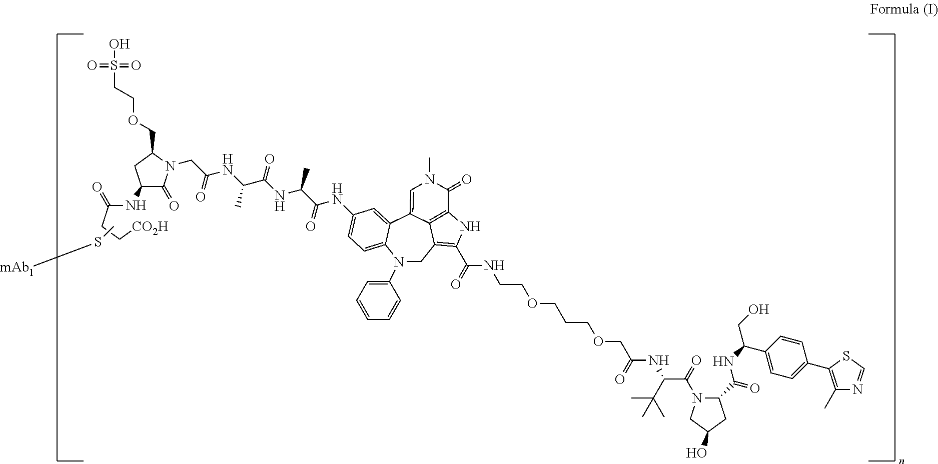

wherein n is an integer from 1 to 10, and wherein mAb1 is an IgG₁ anti-human CD33 antibody comprising a heavy chain variable region comprising a CDR-H1, a CDR-H2, and a CDR-H3; and a light chain variable region comprising a CDR-L1, a CDR-L2, and a CDR-L3; and wherein:
CDR-H1 has the amino acid sequence of SEQ ID NO: 1;
CDR-H2 has the amino acid sequence of SEQ ID NO: 2;
CDR-H3 has the amino acid sequence of SEQ ID NO: 3;
CDR-L1 has the amino acid sequence of SEQ ID NO: 4;
CDR-L2 has the amino acid sequence of SEQ ID NO: 5; and
CDR-L3 has the amino acid sequence of SEQ ID NO: 6.

Another aspect comprises the anti-human CD33 BETd ADC of the first aspect, wherein mAb1 comprises: a heavy chain variable region having the amino acid sequence of SEQ ID NO: 7; and a light chain variable region having the amino acid sequence of SEQ ID NO: 8.

Another aspect comprises anti-human CD33 BETd ADC of the first or second aspect, wherein mAb1 comprises: a heavy chain having the amino acid sequence of SEQ ID NO: 9 or SEQ ID NO: 10; and a light chain having the amino acid sequence of SEQ ID NO: 11.

Another aspect comprises the anti-human CD33 BETd ADC of the first through third aspects, wherein n is about 2.

Another aspect comprises the anti-human CD33 BETd ADC of the first through third aspects, wherein 296C of SEQ ID NO:9 or SEQ ID NO: 10 is the site of conjugation.

Another aspect comprises anti-human CD33 BETd ADC comprising the following structure:

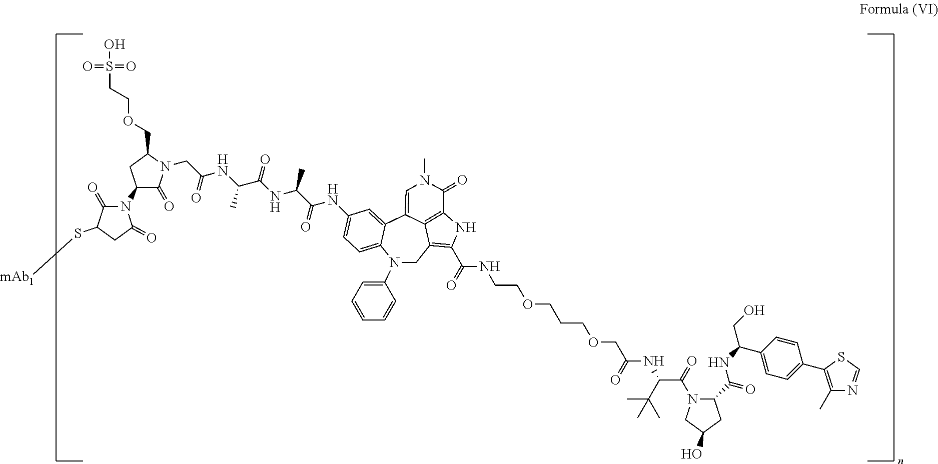

wherein n is about 2, and wherein mAb1 is an IgG₁ anti-human CD33 antibody comprising: a heavy chain having the amino acid sequence of SEQ ID NO: 9 or SEQ ID NO: 10; and a light chain having the amino acid sequence of SEQ ID NO: 11.

Another aspect comprises the anti-human CD33 BETd ADC of the sixth aspect, wherein 296C is the site of conjugation.

Another aspect comprises a compound of Formula (III):

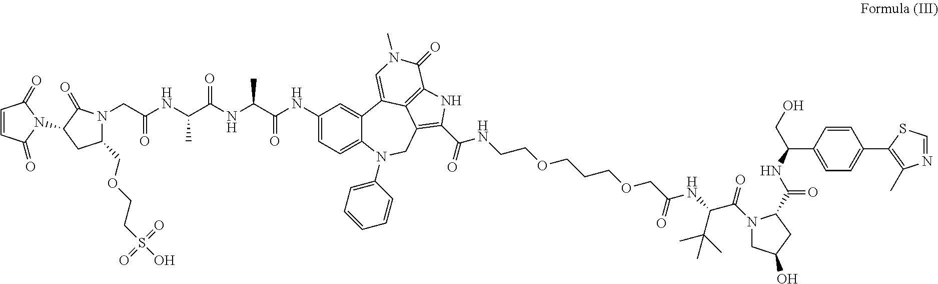 [SMILES: Cc1ncsc1-c1ccc([C@H](CO)NC(=O)[C@@H]2C[C@@H](O)CN2C(=O)[C@@H](NC(=O)COCCCOCCNC(=O)c2[nH]c3c(=O)n(C)cc4c3c2CN(c2ccccc2)c2ccc(NC(=O)[C@H](C)NC(=O)[C@H](C)NC(=O)CN3C(=O)[C@@H](N5C(=O)C=CC5=O)C[C@H]3COCCS(=O)(=O)O)cc2-4)C(C)(C)C)cc1]

Another aspect comprises a pharmaceutical composition comprising the anti-human CD33 BETd ADC of any of first through seventh aspects and further comprises a pharmaceutically acceptable carrier.

Another aspect comprises a method of treating acute myeloid leukemia, the method comprising: administering the anti-human CD33 BETd ADC according to any of the first through seventh aspects to a patient in need thereof.

Another aspect comprises a method of treating acute myeloid leukemia, the method comprising: administering the pharmaceutical composition of the ninth aspect to a patient in need thereof.

Another aspect comprises polynucleotide comprising a nucleotide sequence encoding an anti-human CD33 antibody, wherein the antibody comprises a heavy chain variable region comprising a CDR-H1, a CDR-H2, and a CDR-H3; and a light chain variable region comprising a CDR-L1, a CDR-L2, and a CDR-L3; and wherein:
CDR-H1 has the amino acid sequence of SEQ ID NO: 1;
CDR-H2 has the amino acid sequence of SEQ ID NO: 2;
CDR-H3 has the amino acid sequence of SEQ ID NO: 3;
CDR-L1 has the amino acid sequence of SEQ ID NO: 4;
CDR-L2 has the amino acid sequence of SEQ ID NO: 5; and
CDR-L3 has the amino acid sequence of SEQ ID NO: 6.

Another aspect comprises a method for producing an anti-human CD33 bromo- and extra-terminal domain degrader (BETd) antibody-drug conjugate (ADC) comprising the structure of Formula (I), wherein n is an integer from 1 to 10, and wherein mAb1 is an IgG₁ anti-human CD33 antibody comprising: a heavy chain having the amino acid sequence of SEQ ID NO: 9 or SEQ ID NO: 10; and a light chain having the amino acid sequence of SEQ ID NO: 11, wherein the method comprises reducing an antibody with an excess of reducing agent and then conjugating the antibody with an excess of drug-linker.

### BRIEF DESCRIPTION OF THE DRAWINGS

FIG. 1 depicts the results of mAb1-LD3 Activity in Patient Derived Xenograft (“PDX”) Systemic Models.

FIG. 2 depicts efficacy results displayed by mAb1-LD3 in CDX mouse models.

### DETAILED DESCRIPTION

Disclosed herein are embodiments of bromo- and extra-terminal domain degraders (BETd) and anti-human CD33 BETd ADCs comprising a BETd. Certain embodiments also include linker-drugs useful for synthesizing the anti-human CD33 BETd ADCs, methods of making the anti-human CD33 BETd ADCs, and methods of using the anti-human CD33 BETd ADCs.

### 1.1. BET Degraders (Structural Formula II)

BET proteins play a crucial role in regulating gene transcription through epigenetic interactions between bromodomains and acetylated histones during cellular proliferation and differentiation. Presented herein is a BET degrader drug (BETd drug) according to Formula (II), which may be purposed for targeted delivery to cells by conjugation to an anti-human CD33 antibody.

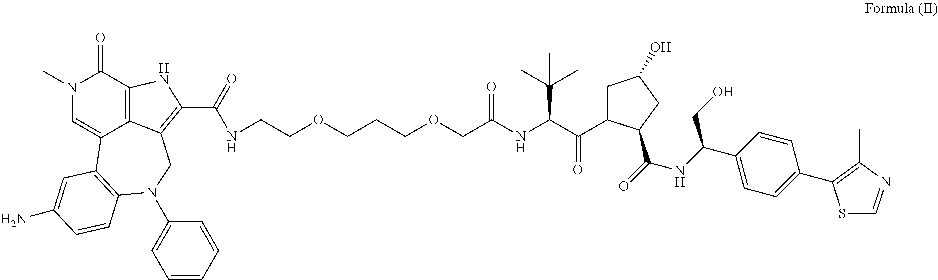 [SMILES: Cc1ncsc1-c1ccc([C@H](CO)NC(=O)[C@@H]2C[C@@H](O)CC2C(=O)[C@@H](NC(=O)COCCCOCCNC(=O)c2[nH]c3c(=O)n(C)cc4c3c2CN(c2ccccc2)c2ccc(N)cc2-4)C(C)(C)C)cc1]

In embodiments, the BETd drug is a compound according to Formula (II). In embodiments, the BETd drug is N-[(3-{2-[(10-amino-2-methyl-3-oxo-7-phenyl-3,4,6,7-tetrahydro-2H-2,4,7-triazadibenzo[cd, f]azulene-5-carbonyl)amino]ethoxy}propoxy)acetyl]-3-methyl-L-valyl-(4R)-4-hydroxy-N-{(1R)-2-hydroxy-1-[4-(4-methyl-1,3-thiazol-5-yl)phenyl]ethyl}-L-prolinamide. 1.2. BETd Linker Drugs (Structural Formula III)

In certain embodiments, the BETd linker drug is a compound according to Formula (III):

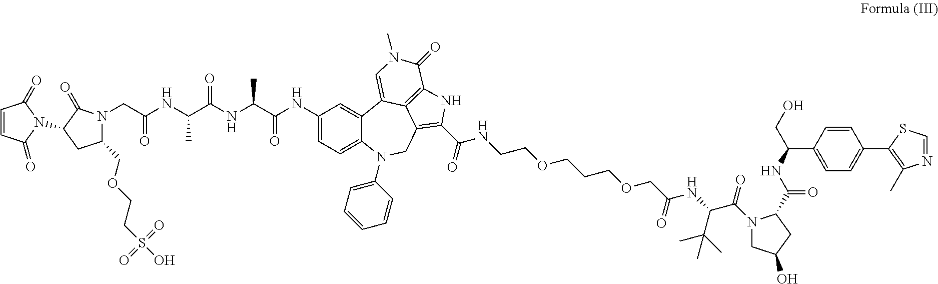 [SMILES: Cc1ncsc1-c1ccc([C@H](CO)NC(=O)[C@@H]2C[C@@H](O)CN2C(=O)[C@@H](NC(=O)COCCCOCCNC(=O)c2[nH]c3c(=O)n(C)cc4c3c2CN(c2ccccc2)c2ccc(NC(=O)[C@H](C)NC(=O)[C@H](C)NC(=O)CN3C(=O)[C@@H](N5C(=O)C=CC5=O)C[C@H]3COCCS(=O)(=O)O)cc2-4)C(C)(C)C)cc1]

In embodiments, the BETd linker drug is N-[(3-{2-[(10-{[N-({(3S,5S)-3-(2,5-dioxo-2,5-dihydro-1H-pyrrol-1-yl)-2-oxo-5-[(2-sulfoethoxy)methyl]pyrrolidin-1-yl}acetyl)-L-alanyl-L-alanyl]amino}-2-methyl-3-oxo-7-phenyl-3,4,6,7-tetrahydro-2H-2,4,7-triazadibenzo[cd, f]azulene-5-carbonyl)amino]ethoxy}propoxy)acetyl]-3-methyl-L-valyl-(4R)-4-hydroxy-N-{(1R)-2-hydroxy-1-[4-(4-methyl-1,3-thiazol-5-yl)phenyl]ethyl}-L-prolinamide.

### 1.3. Anti-Human CD33 BETd ADCs

BET degraders as described herein may be conjugated to an anti-human CD33 antibody to form an anti-human CD33 BETd ADC. ADCs selectively deliver one or more drug moiety(s) to target tissues, such as CD33 expressing tumors. Thus, in embodiments, the present disclosure provides anti-human CD33 BETd ADCs for therapeutic use in the treatment of acute myeloid leukemia.

In embodiments of the anti-human CD33 BETd ADCs described herein, BETd drugs are conjugated to the anti-human CD33 antibody by way of a linker moiety. In embodiments, the linker moiety connects the BETd drug to the anti-human CD33 antibody by forming a covalent linkage to the BETd drug at one location and a covalent linkage to the anti-human CD33 antibody at another. The covalent linkages are formed by reaction between functional groups on the linker and functional groups on the BETd drug and the anti-human CD33 antibody. In certain embodiments, the anti-human CD33 ADC of the present disclosure is comprised of an anti-human CD33 antibody conjugated to the BETd linker drug of Formula (III).

1.3.1. Antibody mAb1

In certain embodiments, mAb1 is a humanized recombinant IgG₁ that targets the extracellular domain of CD33, resulting in CD33 binding, which may also be referred to as an anti-human CD33 antibody.

In certain embodiments, mAb1 comprises variable regions and CDRs (complementary determining regions) identified according to rules developed in the art and/or by aligning sequences against a database of known variable regions.

Methods for identifying these regions are described in Kontermann and Dubel, eds., Antibody Engineering, Springer, New York, N.Y., 2001 and Dinarello et al., Current Protocols in Immunology, John Wiley and Sons Inc., Hoboken, N.J., 2000. For example, CDRs may be identified in accordance with one of the schemes provided by Kabat et al. (1991) Sequences of Proteins of Immunological Interest (5^{th} Ed.), U.S. Dept. of Health and Human Services, PHS, NIH, NIH Publication No. 91-3242 (referred to herein as “Kabat”); or in accordance with AbM (Oxford Molecular/MSI Pharmacopia) (referred to herein as “AbM”). AbM can be obtained from the Abysis database at www.bioinf.org.uk/abs (maintained by A. C. Martin in the Department of Biochemistry & Molecular Biology University College London).

In embodiments, mAb1 comprises a CDR-H1 having the amino acid sequence of SEQ ID NO: 1, a CDR-H2 having the amino acid sequence of SEQ ID NO: 2; a CDR-H3 having the amino acid sequence of SEQ ID NO: 3, a CDR-L1 having the amino acid sequence of SEQ ID NO: 4, a CDR-L2 having the amino acid sequence of SEQ ID NO: 5; and a CDR-L3 having the amino acid sequence of SEQ ID NO: 6.

In embodiments, mAb1 comprises a heavy chain variable region comprising the amino acid sequence of SEQ ID NO: 7: EVQLVESGGGVVQPGRSLRLSCAASGFTLSDYAMAWVRQAPGKGLEWVATISYDAGR TYYRDAVKGRFTISRDNSKNTLYLQMNSLRAEDTAVYYCERPPGVYYGSYWGQGTMV TVSS (SEQ ID NO: 7); and a light chain variable region comprising the amino acid sequence of SEQ ID NO: 8: DIQMTQSPSSLSASVGDRVTITCRASQDISNFLNWYQQKPGKVPKLLIYYTSNLQSGVPS RFSGSGSGTDFTLTISSLQPEDVATYYCQQYDESPPTFGQGTKLEIK (SEQ ID NO: 8).

In embodiments, mAb1 comprises a heavy chain sequence comprising the amino acid sequence of SEQ ID NO: 9 (constant regions are bold; CDRs are underlined (disclosed as SEQ ID NOS: 1-3, respectively, in order of appearance); CH2 mutation=Y296C/N297A is italicized and underlined):

EVQLVESGGG VVQPGRSLRL SCAASGFTLS DYAMAWVRQA PGKGLEWVAT
ISYDAGRTYYRDAVKGRFTI SRDNSKNTLY LQMNSLRAED TAVYYCERPP
GVYYGSYWGQ GTMVTVSSAS TKGPSVFPLA PSSKSTSGGT AALGCLVKDY
FPEPVTVSWN SGALTSGVHT FPAVLQSSGL YSLSSVVTVP SSSLGTQTYI
CNVNHKPSNT KVDKKVEPKS CDKTHTCPPC PAPELLGGPS VFLFPPKPKD
TLMISRTPEV TCVVVDVSHE DPEVKFNWYV DGVEVHNAKTKPREEQCAST
YRVVSVLTVLHQDWLNGKEYKCKVSNKALP APIEKTISKAKGQPREPQVY
TLPPSREEMTKNQVSLTCLV KGFYPSDIAV EWESNGQPEN NYKTTPPVLD
SDGSFFLYSK LTVDKSRWQQGNVFSCSVMHEALHNHYTQKSLSLSPGK
(full-length sequence disclosed as SEQ ID NO: 9).

In embodiments, the predominant species of the heavy chain of mAb1 comprises a heavy chain sequence w ich lacks a C-terminal lysine that comprises a heavy chain sequence comprising the amino acid sequence of SEQ ID NO: 10 (constant regions are bold; CDRs are underlined (disclosed as SEQ ID NOS: 1-3, respectively, in order of appearance); CH2 mutation=Y296C/N297A is italicized and underlined):

EVQLVESGGG VVQPGRSLRL SCAASGFTLS DYAMAWVRQA PGKGLEWVAT
ISYDAGRTYYRDAVKGRFTI SRDNSKNTLY LQMNSLRAED TAVYYCERPP
GVYYGSYWGQ GTMVTVSSAS TKGPSVFPLA PSSKSTSGGT AALGCLVKDY
FPEPVTVSWN SGALTSGVHT FPAVLQSSGL YSLSSVVTVP SSSLGTQTYI
CNVNHKPSNT KVDKKVEPKS CDKTHTCPPC PAPELLGGPS VFLFPPKPKD
TLMISRTPEV TCVVVDVSHE DPEVKFNWYV DGVEVHNAKTKPREEQCAST
YRVVSVLTVLHQDWLNGKEYKCKVSNKALP APIEKTISKAKGQPREPQVY
TLPPSREEMTKNQVSLTCLV KGFYPSDIAV EWESNGQPEN NYKTTPPVLD
SDGSFFLYSK LTVDKSRWQQGNVFSCSVMHEALHNHYTQKSLSLSPG
(full-length sequence disclosed as SEQ ID NO: 10)

and a light chain of mAb1 comprising the amino acid sequence of SEQ ID NO: 11 (constant regions are bold; CDRs are underlined (disclosed as SEQ ID NOS: 4-6, respectively, in order of appearance)):

DIQMTQSPSS LSASVGDRVTITCRASQDISNFLNWYQQKP GKVPKLLIYY
TSNLQSGVPS RFSGSGSGTD FTLTISSLQP EDVATYYCQQYDESPPTFGQ
GTKLEIKRTVAAPSVFIFPP SDEQLKSGTASVVCLLNNFYPREAKVQWKV
DNALQSGNSQESVTEQDSKDSTYSLSSTLT LSKADYEKHKVYACEVTHQG
LSSPVTKSFNRGEC (full-length sequence disclosed as SEQ ID NO: 11).

In embodiments, the heavy chain of mAb1 is encoded by the following nucleotide sequence (full-length sequence disclosed as SEQ ID NO: 12):

(SEQ ID NO: 12)
atggagttcggcctcagctggctgtttctggtcgctattctgaaagg
cgtgcagtgtgaagtccagctggtggagtccggaggaggagtggt
ccagcccggaaggtctctgaggctgagctgtgccgctagcggattta
cactgagcgactacgccatggcttgggtcagacaagcccccgg
caaaggactggagtgggtggctacaatctcctacgacgctggaagga
catactatagggacgccgtgaaaggaaggttcacaatctctag
ggacaactccaagaacacactgtacctccagatgaactctctgaga
gccgaggatactgccgtctactactgcgaaaggcctcccggcgtc
tactatggcagctactggggacaaggcacaatggtgactgtgtcca
gcgctagcacaaaaggcccatccgtctttcctctggcccctagca
gcaaaagcacttccggcggaactgctgctctcggatgtctggtcaa
ggattacttcccagagccagtgacagtgagctggaacagcggag
ctctcacttccggcgtgcatactttcccagccgtcctccaatccag
cggactgtattctctgtcctccgtggtcactgtgccttccagcagcctc
ggcacacagacttacatctgcaatgtgaatcacaagccttccaacac
aaaggtcgacaagaaggtcgagcctaaaagctgtgacaagactc
acacatgccctccttgtccagctccagagctgctgggcggaccatcc
gtgttcctcttccctcctaagcctaaggacactctgatgatctctag
gactccagaggtcacatgtgtcgtggtcgacgtcagccacgaggacc
cagaggtgaaattcaactggtacgtcgacggcgtggaggtgc
acaatgccaaaactaagcctagggaggagcaatgtgccagcacatat
agggtcgtgtccgtgctgacagtgctgcaccaagactggctca
acggaaaggagtacaagtgtaaggtctccaataaggccctcccagct
ccaatcgagaagactatctccaaggccaagggccagcctagg
gaaccacaagtgtacactctcccaccttctagggaagagatgacaa
agaaccaagtgtctctgacttgtctcgtcaagggcttctacccaag
cgacattgctgtggagtgggaaagcaacggccagccagaaaacaa
ctacaagacaacaccaccagtgctcgacagcgatggcagcttct
tcctctactccaagctgacagtggacaagtctaggtggcagcaagg
caacgtgttcagctgcagcgtcatgcacgaagctctccacaatca
ctacactcagaagtctctgtctctgtcccccggcaagtga.

In embodiments, the light chain of mAb1 is encoded by the following nucleotide sequence (full-length sequence disclosed as SEQ ID NO: 13):

(SEQ ID NO: 13)
atggacatgcgcgtgcccgcccagctgctgggcctgctgctgctgt
ggttccccggctcgcgatgcgacatccagatgacccagtctccat
cctcactgtctgcatctgtaggagacagagtcaccatcacttgtcg
ggcgagtcaggatattagcaatttcttaaattggtatcagcagaaacc
agggaaagtccctaagctcctgatctattatacatccaacttgcaa
agtggggtcccatcaaggttcagcggcagtggatctgggacagattt
cactctcaccatcagcagcctgcagcctgaagatgttgcaacttatt
actgccaacagtatgacgagagccctcccacttttggccagggga
ccaagctggagatcaaacgtacggtggctgcaccatctgtcttcatc
ttcccgccatctgatgagcagttgaaatctggaactgcctctgttgt
gtgcctgctgaataacttctatcccagagaggccaaagtacagtgga
aggtggataacgccctccaatcgggtaactcccaggagagtgtc
acagagcaggacagcaaggacagcacctacagcctcagcagcaccc
tgacgctgagcaaagcagactacgagaaacacaaagtctac
gcctgcgaagtcacccatcagggcctgagctcgcccgtcacaaagag
cttcaacaggggagagtgttga

### 1.3.2. Number of Linked Drugs

The ADCs disclosed herein comprise drug molecules linked to antibody moieties in various stoichiometric molar ratios depending on the configuration of the antibody and, at least in part, the method used to effect conjugation.

The terms “drug load” or “drug loading” refer to the number of drug molecules per antibody in an individual ADC molecule. The number of BETd drugs linked to an anti-human CD33 antibody can vary and will be limited by the number of available attachments sites on the anti-human CD33 antibody. As contemplated for the anti-human CD33 BETd ADCs disclosed herein, a linker will link a single BETd drug to the anti-human CD33 antibody to comprise an anti-human CD33 ADC. As long as the anti-human CD33 BETd ADC does not exhibit unacceptable levels of aggregation under the conditions of use and/or storage, anti-human CD33 BETd ADCs (i.e., Formula (I)) having an n of up to 10 are contemplated. In some embodiments, the anti-human CD33 BETd ADCs have an n of in the range of from 1-10. In embodiments, the anti-human CD33 BETd ADCs have an n selected from 1, 2, 3, 4, 5, 6, 7, 8, 9, or 10. In embodiments, n is 2, 4, 6, 8, or 10. In embodiments, n is 2. In embodiments, n is 4. In embodiments, n is 6. In embodiments, the drug loading may comprise 1 drug molecule, 2 drug molecules, 3 drug molecules, 4 drug molecules, 5 drug molecules, 6 drug molecules, 7 drug molecules, 8 drug molecules, 9 drug molecules, or 10 drug molecules.

Provided herein is a conjugation method of producing an anti-human CD33 BETd ADC composition with the predominant species of ADC having an n of 2, and with the composition having a drug-antibody ratio (DAR) of 2 or about 2. The DAR is the average number of drugs linked to each antibody in the composition. Other methods of conjugation are known in the art and can be utilized to produced ADCs having different numbers of conjugated drugs (with an n of, e.g., 1, 2, 3, 4, 5, 6, or 7) and compositions of different DARs (e.g., a DAR of 1, 2, 3, or about 1, about 2, or about 3).

### 1.3.3. Exemplary Anti-Human CD33 BETd ADCs

In embodiments, the anti-human CD33 BETd ADC comprises an anti-human CD33 antibody conjugated to a BETd drug through a cleavable alanine-alanine (aa) linker, where the anti-human CD33 antibody comprises six complementarity determining regions (CDRs) corresponding to the CDRs of mAb1. In embodiments, the linker comprises a maleimide functional group for conjugation to a sulfhydryl of a reduced cysteine from the anti-human CD33 BETd antibody of the ADC.

In embodiments, an anti-human CD33 BETd ADC comprises an anti-human CD33 antibody comprising six complementarity determining regions (CDRs) corresponding to the CDRs of antibody mAb1, which is conjugated to linker-drug LD1 (Formula (III)) via a linkage formed with a sulfhydryl group of a cysteine residue of the anti-human CD33 antibody. In embodiments, the anti-human CD33 antibody comprises the variable heavy (VH) chain and variable light (VL) chain region sequences of antibody mAb1. In embodiments, the anti-human CD33 antibody comprises the heavy chain (HC) and light chain (LC) sequences of antibody mAb1.

In embodiments, an anti-human CD33 BETd ADC has the following Formula (IV):

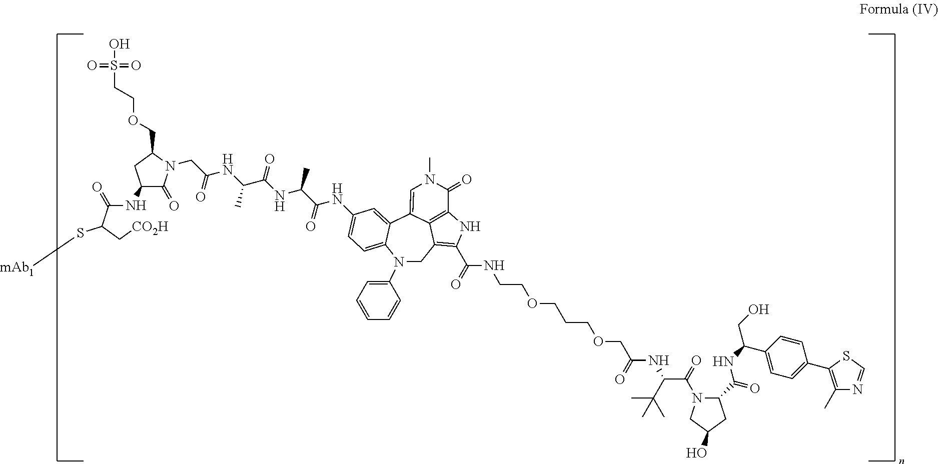

wherein n is an integer from 1-10, and wherein mAb1 is an IgG₁ anti-human CD33 antibody comprising a heavy chain CDR1 of SEQ ID NO: 1, a heavy chain CDR2 of SEQ ID NO: 2, a heavy chain CDR3 of SEQ ID NO: 3, a light chain CDR1 of SEQ ID NO: 4, a light chain CDR2 of SEQ ID NO: 5, and a light chain CDR3 of SEQ ID NO: 6. In embodiments, the antibody mAb1 is an IgG₁ anti-human CD33 antibody comprising a heavy chain variable region comprising the amino acid sequence of SEQ ID NO: 7 and a light chain variable region comprising the amino acid sequence of SEQ ID NO: 8. In embodiments, the antibody Ab is an anti-human CD33 antibody comprising a heavy chain comprising the amino acid sequence of SEQ ID NO: 9 or SEQ ID NO: 10 and a light chain comprising the amino acid sequence of SEQ ID NO: 11. In embodiments, conjugation of the linker-drug to the antibody is via a linkage formed with a sulfhydryl group of a cysteine residue of the antibody. In embodiments, n has a value of 1, 2, 3, 4, 5, 6, 7, 8, 9, or 10. In embodiments, n has a value of 2, 4, 6, 8, or 10. In embodiments, n is 2.

In certain embodiments, an anti-human CD33 BETd ADC comprises an anti-human CD33 antibody comprising six complementarity determining regions (CDRs) corresponding to the CDRs of antibody mAb1, which is conjugated to linker-drug LD1 (Formula (III)) via a linkage formed with a sulfhydryl group of a cysteine residue of the anti-human CD33 antibody. In some embodiments, the anti-human CD33 antibody comprises the variable heavy (VH) chain and variable light (VL) chain region sequences of antibody mAb1. In some embodiments, the anti-human CD33 antibody comprises the heavy chain (HC) and light chain (LC) sequences of antibody mAb1.

In some embodiments, an anti-human CD33 BETd ADC has the following structural Formula (V):

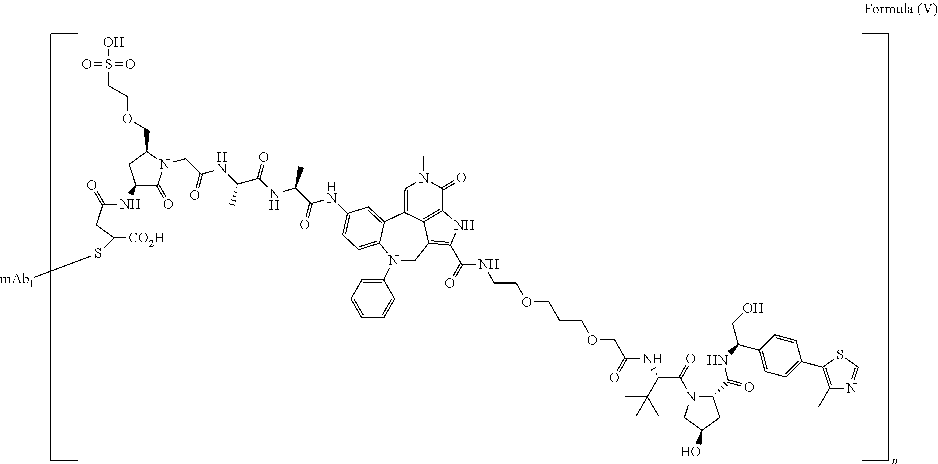

wherein n is an integer from 1-10, and wherein mAb1 is an IgG₁ anti-human CD33 antibody comprising a heavy chain CDR1 of SEQ ID NO: 1, a heavy chain CDR2 of SEQ ID NO: 2, a heavy chain CDR3 of SEQ ID NO: 3, a light chain CDR1 of SEQ ID NO: 4, a light chain CDR2 of SEQ ID NO: 5, and a light chain CDR3 of SEQ ID NO: 6. In embodiments, the antibody mAb1 is an IgG₁ anti-human CD33 antibody comprising a heavy chain variable region comprising the amino acid sequence of SEQ ID NO: 7 and a light chain variable region comprising the amino acid sequence of SEQ ID NO: 8. In embodiments, the antibody Ab is an anti-human CD33 antibody comprising a heavy chain comprising the amino acid sequence of SEQ ID NO: 9 or SEQ ID NO: 10 and a light chain comprising the amino acid sequence of SEQ ID NO: 11. In embodiments, conjugation of the linker-drug to the antibody is via a linkage formed with a sulfhydryl group of a cysteine residue of the antibody. In embodiments, n has a value of 1, 2, 3, 4, 5, 6, 7, 8, 9, or 10. In embodiments, n has a value of 2, 4, 6, 8, or 10. In embodiments, n is 2.

In certain embodiments, an anti-human CD33 BETd ADC comprises an anti-human CD33 antibody comprising six complementarity determining regions (CDRs) corresponding to the CDRs of antibody mAb1, which is conjugated to linker-drug LD1 (Formula (III)) via a linkage formed with a sulfhydryl group of a cysteine residue of the anti-human CD33 antibody. In some embodiments, the anti-human CD33 antibody comprises the variable heavy (VH) chain and variable light (VL) chain region sequences of antibody mAb1. In some embodiments, the anti-human CD33 antibody comprises the heavy chain (HC) and light chain (LC) sequences of antibody mAb1.

In some embodiments, an anti-human CD33 BETd ADC has the following Formula (VI):

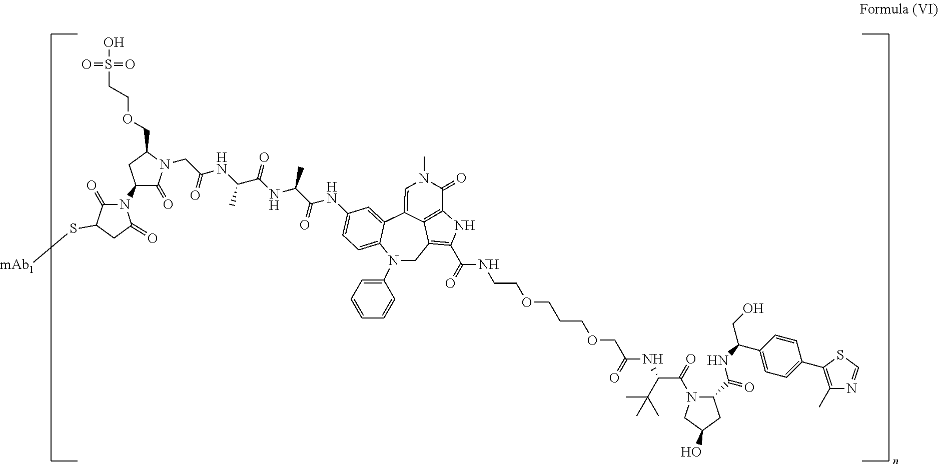

wherein n is an integer from 1-10, and wherein mAb1 is an IgG₁ anti-human CD33 antibody comprising a heavy chain CDR1 of SEQ ID NO: 1, a heavy chain CDR2 of SEQ ID NO: 2, a heavy chain CDR3 of SEQ ID NO: 3, a light chain CDR1 of SEQ ID NO: 4, a light chain CDR2 of SEQ ID NO: 5, and a light chain CDR3 of SEQ ID NO: 6. In embodiments, the antibody mAb1 is an IgG₁ anti-human CD33 antibody comprising a heavy chain variable region comprising the amino acid sequence of SEQ ID NO: 7 and a light chain variable region comprising the amino acid sequence of SEQ ID NO: 8. In embodiments, the antibody Ab is an anti-human CD33 antibody comprising a heavy chain comprising the amino acid sequence of SEQ ID NO: 9 or SEQ ID NO: 10 and a light chain comprising the amino acid sequence of SEQ ID NO: 11. In embodiments, conjugation of the linker-drug to the antibody is via a linkage formed with a sulfhydryl group of a cysteine residue of the antibody. In embodiments, n has a value of 1, 2, 3, 4, 5, 6, 7, 8, 9, or 10. In embodiments, n has a value of 2, 4, 6, 8, or 10. In embodiments, n is 2.

Embodiments of the anti-human CD33 BETd ADCs of this disclosure may be provided as a composition suitable for administration to a subject. In some embodiments, the composition is a pharmaceutical composition, comprising an anti-human CD33 BETd ADC of the present disclosure and a pharmaceutically acceptable carrier.

### 1.4. Methods of Use

Provided herein are embodiments of methods for treating acute myeloid leukemia (AML) comprising administering to a subject in need thereof a therapeutically effective amount of the anti-human CD33 BETd ADCs disclosed herein.

The term “subject,” as used herein, refers to a human. The terms “human,” “patient,” and “subject” are used interchangeably herein.

The terms “treat,” “treating,” and “treatment,” as used herein refer to a method of alleviating or abrogating a disease and/or its attendant symptoms.

The phrase “therapeutically effective amount” refers to an amount sufficient to alleviate to some extent one or more of the symptoms of the condition or disorder being treated when administered for treatment in a particular subject or subject population.

### EXAMPLES

The following Examples highlight certain features and properties of the embodiments described herein.

### Abbreviations

APCI for atmospheric pressure chemical ionization; DCI for desorption chemical ionization; DMSO for dimethyl sulfoxide; ESI for electrospray ionization; HATU for 1-((dimethylamino)(dimethyliminio)methyl)-1H-[1,2,3]triazolo[4,5-b]pyridine 3-oxide hexafluorophosphate(V); HPLC for high performance liquid chromatography; MS for mass spectrum; m/z for mass to charge ratio; NMR for nuclear magnetic resonance; ppm for parts per million; and psi for pounds per square inch.

### Preparative HPLC Conditions—Method A

Samples were purified by preparative HPLC using a Phenomenex® Luna® preparative column (10 μm, C18(2), 250 mm×50 mm, 100 Å) at a flow rate of 100 mL/minute eluted with 20-100% acetonitrile in 0.1% trifluoroacetic acid/water over 30 minutes. Product-containing fractions were collected and freeze-dried to give the desired material.

### Example 1. BETd1

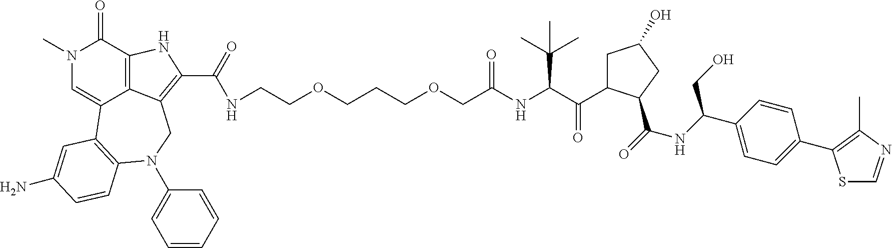 [SMILES: Cc1ncsc1-c1ccc([C@H](CO)NC(=O)[C@@H]2C[C@@H](O)CC2C(=O)[C@@H](NC(=O)COCCCOCCNC(=O)c2[nH]c3c(=O)n(C)cc4c3c2CN(c2ccccc2)c2ccc(N)cc2-4)C(C)(C)C)cc1]

### N-[(3-{2-1[(10-amino-2-methyl-3-oxo-7-phenyl-3,4,6,7-tetrahydro-2H-2,4,7-triazadibenzo[cd, f]azulene-5-carbonyl)amino]ethoxy}propoxy)acetyl]-3-methyl-L-valyl-(4R)-4-hydroxy-N-{(1R)-2-hydroxy-1-[4-(4-methyl-1,3-thiazol-5-yl)phenyl]ethyl}-L-prolinamide

### (Formula (II), BETd1)

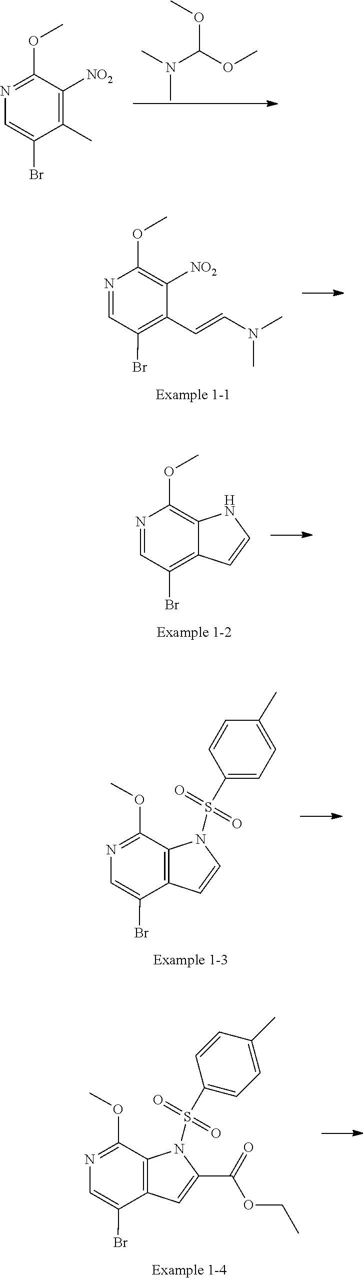

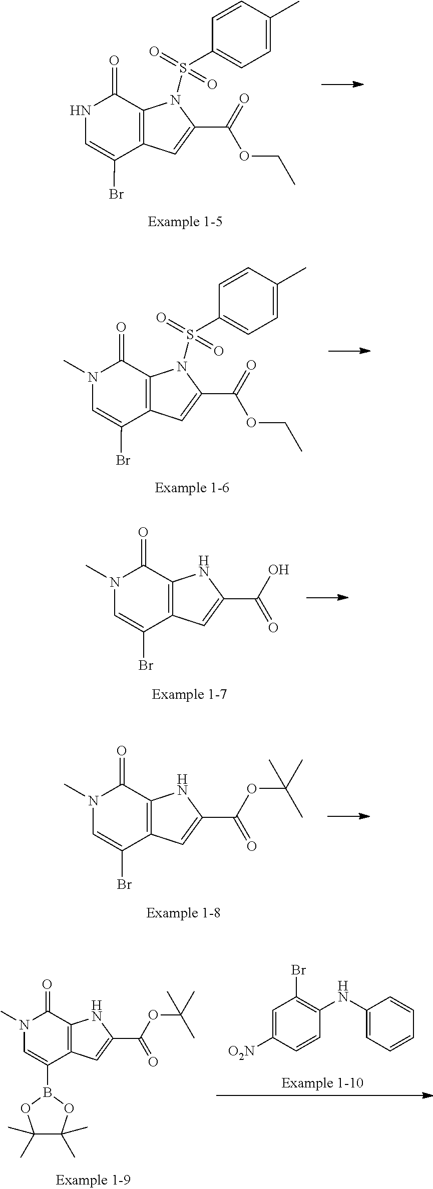

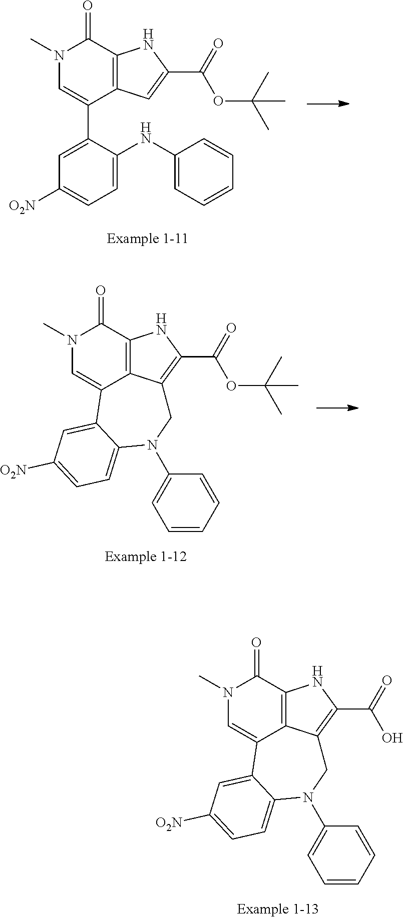 [SMILES: Cn1cc(-c2cc([N+](=O)[O-])ccc2Nc2ccccc2)c2cc(C(=O)OC(C)(C)C)[nH]c2c1=O ; Cn1cc2c3c(c(C(=O)OC(C)(C)C)[nH]c3c1=O)CN(c1ccccc1)c1ccc([N+](=O)[O-])cc1-2 ; Cn1cc2c3c(c(C(=O)O)[nH]c3c1=O)CN(c1ccccc1)c1ccc([N+](=O)[O-])cc1-2]

### Example 1-1

### (E)-2-(5-bromo-2-methoxy-3-nitropyridin-4-yl)-N,N-dimethylethen-1-amine

5-Bromo-2-methoxy-4-methyl-3-nitropyridine (15.0 g, 60.7 mmol) was dissolved in N,N-dimethylformamide (300 mL), and lithium methanolate (6.07 mL, 6.07 mmol, 1 M in methanol) was added. The reaction mixture was heated at 100° C. To this mixture, 1,1-dimethoxy-N,N-dimethylmethanamine (64.5 mL, 486 mmol) was added dropwise over 10 minutes. The reaction mixture was stirred at 95° C. for 16 hours. The reaction mixture was cooled to ambient temperature, and water was added (300 mL, exothermic). The resulting precipitate was collected by vacuum filtration, washed with water, and dried to provide the title compound (13.9 g, 76% yield). ¹H NMR (400 MHz, DMSO-d₆) δ ppm 8.25 (s, 1H), 7.05 (d, J=13.5 Hz, 1H), 4.81 (d, J=13.5 Hz, 1H), 3.88 (s, 3H), 2.91 (s, 6H); MS (ESI⁺) m/z 302.0 (M+H)⁺.

### Example 1-2

### 4-bromo-7-methoxy-1H-pyrrolo[2,3-c]pyridine

Example 1-1 (13.9 g, 45.8 mmol) and ethyl acetate (150 mL) were added to Raney®-nickel 2800 (pre-washed with ethanol), water slurry (6.90 g, 118 mmol) in a stainless steel pressure bottle and stirred for 30 minutes at 30 psi of H₂ and ambient temperature. The reaction mixture was filtered and concentrated. The residue was triturated with dichloromethane and filtered to provide the title compound (5.82 g). The mother liquor was concentrated, and the residue was triturated with dichloromethane and filtered to provide an additional 1.63 g of the title compound (7.45 g total, 72% yield). ¹H NMR (400 MHz, DMSO-d₆) δ ppm 12.15 (s, 1H), 7.76 (s, 1H), 7.56 (t, J=2.8 Hz, 1H), 6.43 (dd, J=2.9, 2.0 Hz, 1H), 4.01 (s, 3H); MS (ESI⁺) m/z 227.0 (M+H)⁺.

### Example 1-3

### 4-bromo-7-methoxy-1-(4-methylbenzene-1-sulfonyl)-1H-pyrrolo[2,3-c]pyridine

A solution of Example 1-2 (7.42 g, 32.7 mmol) in N,N-dimethylformamide (235 mL) was stirred at ambient temperature. Sodium hydride (1.18 g, 1.96 g of 60% dispersion in oil, 49.0 mmol) was added to the solution, and the reaction mixture was stirred for 10 minutes. p-Toluenesulfonyl chloride (9.35 g, 49.0 mmol) was then added portionwise, and the mixture was stirred at ambient temperature under nitrogen for 16 hours. The reaction mixture was quenched carefully with water, vacuum filtrated on a Buchner funnel and washed with water. The title compound was then produced after collection and drying in a vacuum oven at 50° C. (12.4 g, 99% yield). ¹H NMR (400 MHz, DMSO-d₆) δ ppm 8.18 (d, J=3.6 Hz, 1H), 8.00 (s, 1H), 7.90-7.82 (m, 2H), 7.50-7.42 (m, 2H), 6.81 (d, J=3.7 Hz, 1H), 3.82 (s, 3H), 2.39 (s, 3H); MS (APCI⁺) m/z 383.1 (M+H)⁺.

### Example 1-4

### ethyl 4-bromo-7-methoxy-1-(4-methylbenzene-1-sulfonyl)-1H-pyrrolo[2,3-c]pyridine-2-carboxylate

To a solution of Example 1-3 (12 g, 32 mmol) in tetrahydrofuran (150 mL) lithium diisopropyl amide (LDA, 1.95 M, 24.3 mL, 47.2 mmol) was added dropwise at −70° C., and the mixture was stirred at −70° C. to −50° C. for 45 minutes followed by dropwise addition of ethyl carbonochloridate (5.12 g, 47.2 mmol). After 1.5 hours, the reaction mixture was quenched with saturated ammonium chloride solution. Volatiles were removed under reduced pressure, and the residue was extracted with ethyl acetate (3×300 mL). The combined organic fractions were dried over anhydrous sodium sulfate, filtered, and concentrated under reduced pressure to give the crude title compound, which was triturated with dichloromethane and methanol (1:10) to give the title compound (13 g, 91% yield). ¹H NMR (400 MHz, DMSO-d₆) δ ppm 8.17-8.08 (m, 3H), 7.25 (m, 2H), 7.25 (s, 1H), 4.39 (q, J=7.1 Hz, 2H), 3.82 (s, 3H), 2.41 (s, 3H), 1.33 (t, J=7.1 Hz, 3H); MS (ESI⁺) m/z 454.9 (M+H)⁺.

### Example 1-5

### ethyl 4-bromo-1-(4-methylbenzene-1-sulfonyl)-7-oxo-6,7-dihydro-1H-pyrrolo[2,3-c]pyridine-2-carboxylate

Chlorotrimethylsilane (10.43 g, 96 mmol) was added dropwise to a mixture of Example 1-4 (29 g, 64 mmol) and sodium iodide (14.38 g, 96 mmol) in acetonitrile (400 mL). The resulting mixture was stirred at ambient temperature for 1 hour, then water (0.576 g, 32.0 mmol) was added dropwise and the mixture stirred at 65° C. for 3 hours. The reaction mixture was cooled to ambient temperature and filtered. The precipitate was dissolved in dichloromethane, filtered, concentrated, and washed with petroleum ether and dichloromethane to give the title compound (27.9, 99% yield). ¹H NMR (400 MHz, DMSO-d₆) δ ppm 11.76 (s, 1H), 8.27-8.20 (m, 2H), 7.50-7.43 (m, 3H), 6.99 (s, 1H), 4.35 (q, J=7.2 Hz, 2H), 2.39 (s, 3H), 1.31 (t, J=7.1 Hz, 3H); MS (ESI⁺) m/z 440.9 (M+H)⁺.

### Example 1-6

### ethyl 4-bromo-6-methyl-1-(4-methylbenzene-1-sulfonyl)-7-oxo-6,7-dihydro-1H-pyrrolo[2,3-c]pyridine-2-carboxylate

Iodomethane (3.20 mL, 51.1 mmol) was added dropwise to a mixture of Example 1-5 (18.72 g, 42.6 mmol) and cesium carbonate (16.66 g, 51.1 mmol) in anhydrous N,N-dimethylformamide (200 mL). The reaction mixture was stirred for 72 hours at ambient temperature. Water was added (500 mL), and the precipitate was collected by filtration. The collected material was washed with water and dried overnight under vacuum at 55° C. to provide the title compound (17.9 g, 90% yield). ¹H NMR (400 MHz, DMSO-d₆) δ ppm 8.28 (d, J=8.1 Hz, 2H), 7.91 (s, 1H), 7.50 (d, J=8.1 Hz, 2H), 7.02 (s, 1H), 4.37 (q, J=7.1 Hz, 2H), 3.43 (s, 3H), 2.41 (s, 3H), 1.32 (t, J=7.1 Hz, 3H); MS (ESI⁺) m/z 454.9 (M+H)⁺.

### Example 1-7

### 4-bromo-6-methyl-7-oxo-6,7-dihydro-1H-pyrrolo[2,3-c]pyridine-2-carboxylic acid

A solution of Example 1-6 (35 g, 80 mmol) in dioxane (400 mL) was treated with 1.0 M sodium hydroxide solution (159 mL, 319 mmol), and the mixture was stirred at 80° C. for 4 hours. The reaction mixture was adjusted to pH 1 with 2 N HCl. The precipitate was collected by filtration, washed with water (50 mL), and dried under vacuum to give the title compound (20.8 g, 77 mmol, 96% yield). ¹H NMR (500 MHz, DMSO-d₆) δ ppm 13.17 (s, 1H), 12.99 (s, 1H), 7.60 (s, 1H), 6.77 (s, 1H), 3.49 (s, 3H); MS (DCI⁺) m/z 270.9 (M+H)⁺.

### Example 1-8

### tert-butyl 4-bromo-6-methyl-7-oxo-6,7-dihydro-1H-pyrrolo[2,3-c]pyridine-2-carboxylate

To Example 1-7 (10 g, 36.9 mmol) in dioxane (200 mL), (E)-tert-butyl N,N′-diisopropylcarbamimidate (14.78 g, 73.8 mmol) was added, and the reaction mixture was sealed and heated at 110° C. for 18 hours. Then another batch of (E)-tert-butyl N,N′-diisopropylcarbamimidate (14.78 g, 73.8 mmol) was added, and the reaction mixture was stirred at 110° C. for 24 hours. The reaction mixture was cooled to ambient temperature and partitioned between ethyl acetate (600 mL) and water (100 mL). The aqueous layer was extracted with ethyl acetate (200 mL) again. The combined organic layers were washed with saturated aqueous sodium chloride (100 mL), dried over anhydrous sodium sulfate, filtered and concentrated. The residue was purified by flash chromatography (silica gel, eluted with 25% to 60% ethyl acetate in petroleum ether) to give the title compound (11.7 g, 90% yield). ¹H NMR (500 MHz, DMSO-d₆) δ ppm 13.09 (s, 1H), 7.61 (s, 1H), 6.75 (s, 1H), 3.51 (s, 3H), 1.55 (s, 9H); MS (ESI⁺) m/z 327.1 (M+H)⁺.

### Example 1-9

### tert-butyl 6-methyl-7-oxo-4-(4,4,5,5-tetramethyl-1,3,2-dioxaborolan-2-yl)-6,7-dihydro-1H-pyrrolo[2,3-c]pyridine-2-carboxylate

A mixture of Example 1-8 (9.7 g, 28 mmol)), potassium acetate (8.29 g, 84 mmol), dicyclohexyl(2′,4′,6′-triisopropyl-[1,1′-biphenyl]-2-yl)phosphine (0.537 g, 1.13 mmol), tris(dibenzylideneacetone)dipalladium(0) (0.258 g, 0.282 mmol) and 4,4,4′,4′,5,5,5′,5′-octamethyl-2,2′-bi(1,3,2-dioxaborolane) (21.46 g, 84 mmol) in dioxane (150 mL) was degassed and back-filled with nitrogen several times. The reaction mixture was heated at 80° C. for 16 hours. The reaction mixture cooled to ambient temperature and filtered through a pad of diatomaceous earth. The pad was washed with ethyl acetate and the filtrate was concentrated. The crude material was washed with petroleum ether to provide the title compound (9.0 g, 79% yield). ¹H NMR (400 MHz, DMSO-d₆) δ ppm 12.53 (s, 1H), 7.53 (s, 1H), 7.00 (s, 1H), 3.51 (s, 3H), 1.51 (s, 9H), 1.27 (s, 12H); MS (ESI⁺) m/z 375.4 (M+H)⁺.

### Example 1-10

### 2-bromo-4-nitro-N-phenylaniline

A mixture of 2-bromo-1-fluoro-4-nitrobenzene (30.0 g, 136 mmol), aniline (25.4 g, 273 mmol) and N,N-diisopropylethylamine (47.6 mL, 273 mmol) was stirred at 140° C. under reflux for 12 hours. After cooling to ambient temperature, N,N-diisopropylethylamine was removed under reduced pressure. The residue was diluted with ethyl acetate (500 mL). The resultant mixture was washed sequentially with aqueous 2 N HCl (200 mL) and saturated aqueous sodium chloride (500 mL), and then dried over anhydrous sodium sulfate, filtered and concentrated under reduced pressure. The title compound (25.5 g, 63.3% yield) was obtained after washing with cold methanol (100 mL). ¹H NMR (400 MHz, DMSO-d₆) δ ppm 8.47 (s, 1H), 8.39 (d, J=2.65 Hz, 1H), 8.05 (dd, J=9.26, 2.65 Hz, 1H), 7.39-7.51 (m, 2H), 7.32 (d, J=7.50 Hz, 2H), 7.18-7.26 (m, 1H), 7.02 (d, J=9.26 Hz, 1H); MS (ESI⁺) m/z 293.2 (M+H)⁺.

### Example 1-11

### tert-butyl 4-(2-anilino-5-nitrophenyl)-6-methyl-7-oxo-6,7-dihydro-1H-pyrrolo[2,3-c]pyridine-2-carboxylate

A mixture of Example 1-10 (1.15 g, 3.92 mmol), Example 1-9 (1.762 g, 4.71 mmol), 1,3,5,7-tetramethyl-6-phenyl-2,4,8-trioxa-6-phosphaadamantane (0.134 g, 0.459 mmol), tris(dibenzylideneacetone)dipalladium(0) (0.108 g, 0.118 mmol), and potassium phosphate (0.833 g, 3.92 mmol) in dioxane (24 mL) and water (6 mL) was degassed and back-filled with nitrogen several times. The reaction mixture was heated at 60° C. for 16 hours. The reaction mixture was partitioned between water and ethyl acetate and filtered. The filtrate was separated. The aqueous layer was extracted with additional ethyl acetate 3 times. The combined organic layers were washed with saturated aqueous sodium chloride, dried over anhydrous magnesium sulfate, filtered, and concentrated. The residue was purified by flash chromatography (silica gel eluted with 5% methanol in ethyl acetate) to give a second batch of title compound. Total title compound isolated 1.79 g (99% yield). ¹H NMR (501 MHz, DMSO-d₆) δ ppm 12.72 (s, 1H), 8.08 (dd, J=9.1, 2.7 Hz, 2H), 8.03 (d, J=2.8 Hz, 1H), 7.44 (s, 1H), 7.36-7.29 (m, 2H), 7.22-7.14 (m, 3H), 7.12-7.05 (m, 1H), 6.55 (s, 1H), 3.56 (s, 3H), 1.44 (s, 9H); MS (ESI⁺) m/z 461.3 (M+H)⁺.

### Example 1-12

### tert-butyl 2-methyl-10-nitro-3-oxo-7-phenyl-3,4,6,7-tetrahydro-2H-2,4,7-triazadibenzo[cd,f]azulene-5-carboxylate

A mixture of Example 1-11 (5.2 g, 11.29 mmol) and paraformaldehyde (1.017 g, 33.9 mmol) in acetic acid (100 mL) was stirred at 80° C. for 2 hours. Additional paraformaldehyde (0.678 g, 22.6 mmol) was added, and the reaction mixture was stirred at 80° C. for another 3 hours. The reaction mixture was diluted with water (˜100 mL), and the precipitate was collected by filtration and washed with hexane to give the title compound (5.3 g, 82% yield). MS (APCI⁺) m/z 473.0 (M+H)⁺.

### Example 1-13

### 2-methyl-10-nitro-3-oxo-7-phenyl-3,4,6,7-tetrahydro-2H-2,4,7-triazadibenzo[cd]azulene-5-carboxylic acid

Trifluoroacetic acid (10 mL, 130 mmol) was added to a solution of Example 1-12 (2.6 g, 5.4 mmol) in dichloromethane (20 mL) at ambient temperature, and the resulting mixture was stirred at ambient temperature for 4 hours. The volatiles were removed under reduced pressure. The title compound (2.30 g, 99% yield) was obtained after triturating with water (80 mL), filtering, and drying under high vacuum. ¹H NMR (400 MHz, DMSO-d₆) δ ppm 12.53 (s, 1H), 8.74 (d, J=2.7 Hz, 1H), 8.20 (dd, J=8.7, 2.7 Hz, 1H), 7.98 (s, 1H), 7.57 (d, J=8.7 Hz, 1H), 7.04 (dd, J=8.6, 7.4 Hz, 2H), 6.60 (t, J=7.3 Hz, 1H), 6.48 (d, J=8.0 Hz, 2H), 5.12 (s, 2H), 3.59 (s, 3H); MS (ESI⁺) m/z 417.3 (M+H)⁺.

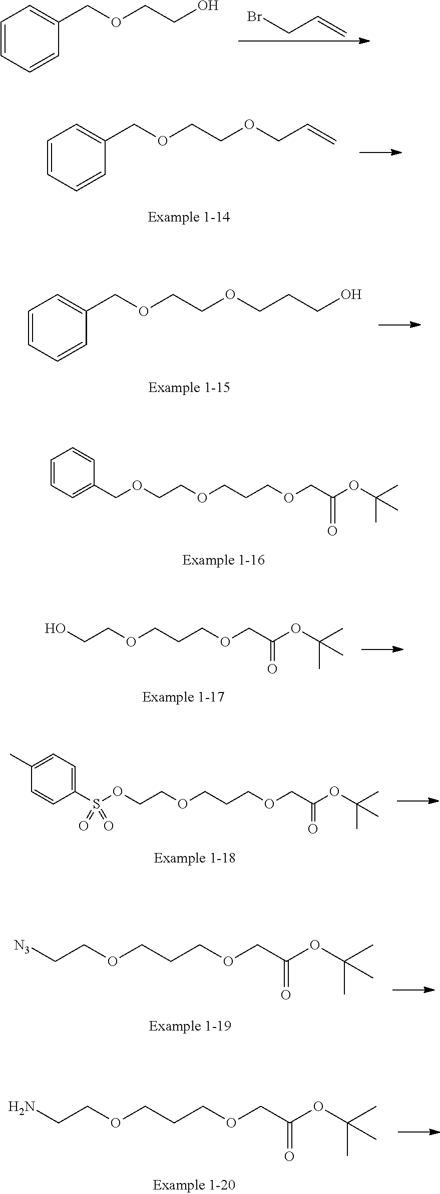

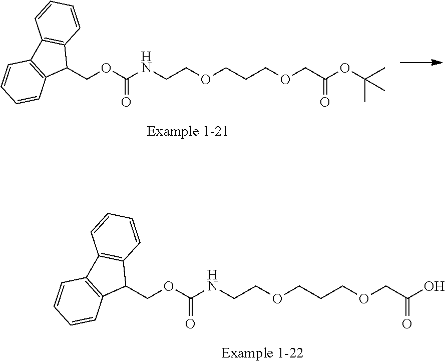 [SMILES: CC(C)(C)OC(=O)COCCCOCCNC(=O)OCC1c2ccccc2-c2ccccc21 ; O=C(O)COCCCOCCNC(=O)OCC1c2ccccc2-c2ccccc21]

### Example 1-14

### ({2-1[(prop-2-en-1-yl)oxy]ethoxy}methyl)benzene

60% sodium hydride (7.88 g, 197 mmol) and 3-bromoprop-1-ene (17.1 mL, 197 mmol) at 0° C. were carefully added to a solution of 2-(benzyloxy)ethanol (23.3 mL, 164 mmol) in tetrahydrofuran (160 mL). The reaction mixture was stirred at ambient temperature for 4 hours, quenched with water, and partitioned with ethyl acetate. The organic layer was washed with saturated aqueous sodium chloride, dried with anhydrous sodium sulfate, filtered and concentrated. The residue was purified by flash chromatography (silica gel, 5-10% ethyl acetate in heptanes) to provide the title compound (27.4 g, 87% yield). ¹H NMR (400 MHz, DMSO-d₆) δ ppm 7.38-7.22 (m, 5H), 5.86 (ddt, J=17.3, 10.5, 5.3 Hz, 1H), 5.23 (dq, J=17.3, 1.8 Hz, 1H), 5.16-5.08 (m, 1H), 4.47 (s, 2H), 3.94 (dt, J=5.3, 1.5 Hz, 2H), 3.59-3.49 (m, 4H).

### Example 1-15

### 3-[2-(benzyloxy)ethoxy]propan-1-ol

Borane-methyl sulfide complex (13.5 mL, 143 mmol) at 0° C. was added to a solution of Example 1-14 (27.4 g, 143 mmol) in toluene (140 mL). The mixture was stirred at ambient temperature for 3 hours and carefully quenched at 0° C. with methanol (140 mL), followed by 30% hydrogen peroxide (19.39 g, 171 mmol), followed by 2 M aqueous sodium hydroxide (178 mL, 356 mmol). The reaction mixture was stirred at ambient temperature for 2 hours and then partitioned between ethyl acetate and water. The organic layer was washed with 10% aqueous sodium bisulfite and saturated aqueous sodium chloride, dried with anhydrous sodium sulfate, filtered, and concentrated under reduced pressure. The residue was purified by flash chromatography (silica gel, 10-40% ethyl acetate in heptanes) to provide the title compound (13.8 g, 46% yield). ¹H NMR (501 MHz, DMSO-d₆) δ ppm 7.34 (dt, J=6.7, 1.1 Hz, 1H), 7.34-7.26 (m, 3H), 7.29-7.22 (m, 1H), 4.47 (s, 2H), 4.37 (t, J=5.2 Hz, 1H), 3.56-3.40 (m, 8H), 1.63 (p, J=6.4 Hz, 2H).

### Example 1-16

### tert-butyl {3-[2-(benzyloxy)ethoxy]propoxy}acetate

A mixture of Example 1-15 (8.9 g, 42.3 mmol), tert-butyl bromoacetate (16.51 g, 85 mmol), tetrabutylammonium chloride (11.76 g, 42.3 mmol) and 10 M aqueous sodium hydroxide (46.6 mL, 466 mmol) in dichloromethane (47.0 mL) was stirred vigorously for 8 hours. Additional tert-butyl bromoacetate (16.51 g, 85 mmol) was added, and the mixture was stirred for 48 hours. The mixture was concentrated under reduced pressure to remove dichloromethane and partitioned between 200 mL each of ethyl acetate and water. The organic layer was washed with 100 mL of saturated aqueous sodium chloride, dried over anhydrous sodium sulfate, filtered, and concentrated. Purification by flash chromatography (silica gel, 120 g column, 5-100% ethyl acetate in heptanes) provided the title compound (9.1 g, 56% yield). ¹H NMR (400 MHz, CDCl₃) δ ppm 1.47 (s, 9H), 1.88-1.95 (m, 2H), 3.57-3.63 (m, 8H), 3.94 (s, 2H), 4.57 (s, 2H), 7.27-7.35 (m, 5H).

### Example 1-17

### tert-butyl[3-(2-hydroxyethoxy)propoxy]acetate

Example 1-16 (11.73 g, 36.2 mmol) in methanol (100 mL) was added to 10% Pd/C, dry (2.30 g, 2.16 mmol) in a 250 mL stainless steel pressure bottle flushed with argon. The reactor was purged with argon and shaken at the standard rate under 20 psi of hydrogen with no external heating for 18 hours. The mixture was filtered through a polypropylene membrane and the filtrate was concentrated under reduced pressure to provide the title compound (8.49 g, 99% yield). ¹H NMR (400 MHz, CDCl₃) δ ppm 1.48 (s, 9H), 1.87-1.93 (m, 2H), 2.36 (br, 1H), 3.56-3.58 (m, 2H), 3.60-3.64 (m, 4H), 3.72-3.74 (m, 2H), 3.96 (s, 2H).

### Example 1-18

### tert-butyl (3-{2-[(4-methylbenzene-1-sulfonyl)oxy]ethoxy}propoxy)acetate

To a solution of Example 1-17 (4.85 g, 20.7 mmol) in pyridine (12 mL) was added (p-toluenesulfonyl chloride (4.34 g, 22.8 mmol). The reaction mixture was stirred for 1 hour and then partitioned between ethyl acetate and water. The organic layer was washed sequentially with cold aqueous 1 M HCl followed by saturated aqueous sodium chloride, dried over anhydrous sodium sulfate, filtered and concentrated. Purification by flash chromatography (silica gel, 5-60% ethyl acetate in heptanes) provided the title compound (5.88 g, 68% yield). ¹H NMR (400 MHz, CDCl₃) δ ppm 1.48 (s, 9H), 1.78-1.85 (m, 2H), 2.45 (s, 3H), 3.49-3.56 (m, 4H), 3.62 (t, J=4.8 Hz, 2H), 3.93 (s, 2H), 4.15 (t, J=4.8 Hz, 2H), 7.35 (d, J=8.0 Hz, 2H), 7.80 (d, J=8.4 Hz, 2H).

### Example 1-19

### tert-butyl[3-(2-azidoethoxy)propoxy]acetate

A mixture of Example 1-18 (5.88 g, 15.14 mmol) and sodium azide (1.181 g, 18.16 mmol) in N,N-dimethylformamide (20 mL) was stirred at 70° C. for 3 hours, cooled, and partitioned between ethyl acetate and water. The organic layer was washed with saturated aqueous sodium chloride, dried over anhydrous sodium sulfate, filtered, and concentrated under reduced pressure. Purification by flash chromatography (silica gel, 5-50% ethyl acetate in heptanes) provided the title compound (3.6 g, 89% yield). ¹H NMR (400 MHz, CDCl₃) δ ppm 1.48 (s, 9H), 1.89-1.94 (m, 2H), 3.36 (t, J=5.0 Hz, 2H), 3.59-3.64 (m, 6H), 3.96 (s, 2H).

### Example 1-20

### tert-butyl[3-(2-aminoethoxy)propoxy]acetate

A mixture of Example 1-19 (3.2 g, 12.3 mmol) and triphenylphosphine (4.86 g, 18.5 mmol) in tetrahydrofuran (36 mL)/water (1.1 mL) was stirred at ambient temperature for 5 hours. The reaction mixture was partitioned between ethyl acetate and water. The organic layer was washed with saturated aqueous sodium chloride, dried over anhydrous sodium sulfate, filtered and concentrated under reduced pressure. Purification by flash chromatography (silica, 1-10% methanol/3% ammonia in dichloromethane) provided the title compound (2.5 g, 87% yield). ¹H NMR (400 MHz, CDCl₃) δ ppm 1.48 (s, 9H), 1.87-1.94 (m, 2H), 2.85 (t, J=5.2 Hz, 2H), 3.45-3.48 (m, 2H), 3.57 (t, J=6.4 Hz, 2H), 3.61 (t, J=6.4 Hz, 2H), 3.95 (s, 2H).

### Example 1-21

### tert-butyl 1-(9H-fluoren-9-yl)-3-oxo-2,7,11-trioxa-4-azatridecan-13-oate

N-ethyl-N-isopropylpropan-2-amine (0.56 mL, 3.2 mmol) was added to a solution of Example 1-20 (0.500 g, 2.143 mmol) in dichloromethane (9 mL). The reaction mixture was cooled to 0° C., and 9-fluorenylmethyl chloroformate (0.61 g, 2.36 mmol) was added dropwise as a solution in dichloromethane (3.0 mL). The ice-water bath was removed, and the reaction mixture was stirred at ambient temperature for 48 hours. The mixture was quenched with saturated aqueous ammonium chloride, and the layers were separated. The aqueous layer was extracted with dichloromethane. The combined organic layers were washed with saturated aqueous sodium bicarbonate and saturated aqueous sodium chloride, dried over anhydrous sodium sulfate, filtered and concentrated under reduced pressure. Purification by flash chromatography (silica gel, 0-50% ethyl acetate in heptanes) provided the title compound (0.64 g, 66% yield). ¹H NMR (400 MHz, DMSO-d₆) δ ppm 7.85 (dt, J=7.5, 0.9 Hz, 2H), 7.65 (d, J=7.4 Hz, 2H), 7.38 (tt, J=7.5, 1.0 Hz, 2H), 7.33-7.22 (m, 3H), 4.25 (d, J=7.0 Hz, 2H), 4.18 (d, J=6.8 Hz, 1H), 3.87 (s, 2H), 3.42 (dt, J=16.1, 6.5 Hz, 4H), 3.33 (s, 1H), 3.09 (q, J=5.9 Hz, 2H), 1.69 (p, J=6.5 Hz, 2H), 1.37 (s, 9H); MS (ESI⁺) m/z 478.1 (M+Na)⁺.

### Example 1-22

### 1-(9H-fluoren-9-yl)-3-oxo-2,7,11-trioxa-4-azatridecan-13-oic acid

Example 1-21 (0.64 g, 1.4 mmol) in formic acid (14 mL) was heated at 60° C. for 2 hours. The volatiles were removed under reduced pressure, and the residue was azeotroped with toluene (3×25 mL) to provide the title compound (0.55 g, 98% yield). ¹H NMR (400 MHz, DMSO-d₆) δ ppm 12.57 (s, 1H), 7.85 (d, J=7.5 Hz, 2H), 7.65 (d, J=7.4 Hz, 2H), 7.38 (td, J=7.5, 1.2 Hz, 2H), 7.33-7.23 (m, 3H), 4.26 (d, J=6.9 Hz, 2H), 4.17 (t, J=6.9 Hz, 1H), 3.93 (s, 2H), 3.45 (d, J=12.8 Hz, 1H), 3.43-3.32 (m, 4H), 3.09 (q, J=5.9 Hz, 2H), 1.70 (p, J=6.5 Hz, 2H); MS (ESI⁺) m/z 400.0 (M+H)⁺.

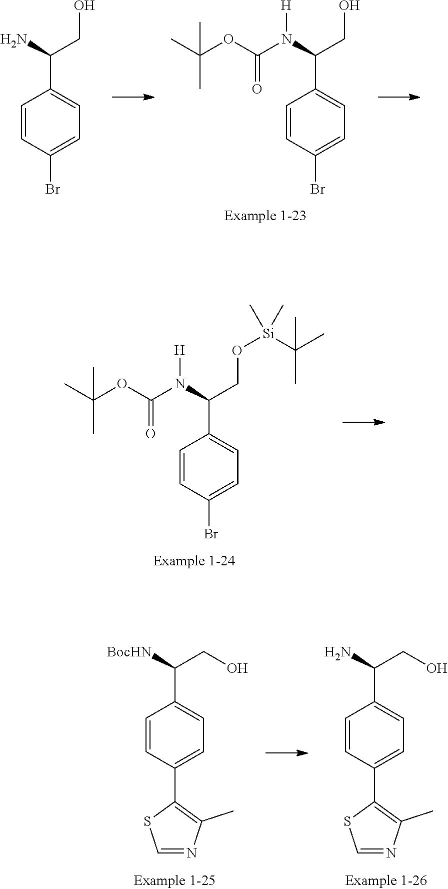 [SMILES: N[C@@H](CO)c1ccc(Br)cc1 ; CC(C)(C)OC(=O)N[C@@H](CO)c1ccc(Br)cc1 ; CC(C)(C)OC(=O)N[C@@H](CO[Si](C)(C)C(C)(C)C)c1ccc(Br)cc1 ; Cc1ncsc1-c1ccc([C@H](CO)NC(=O)OC(C)(C)C)cc1 ; Cc1ncsc1-c1ccc([C@@H](N)CO)cc1]

### Example 1-23

### tert-butyl [(1R)-1-(4-bromophenyl)-2-hydroxyethyl]carbamate

Di-tert-butyl dicarbonate (9.65 mL, 41.6 mmol) and saturated aqueous sodium bicarbonate (50 mL) were added to a solution of (2R)-2-amino-2-(4-bromophenyl)ethan-1-ol hydrochloride (10 g, 39.6 mmol) in tetrahydrofuran (100 mL) at 20° C. The mixture was stirred at 20° C. for 3 hours, quenched with water (150 mL), and extracted with ethyl acetate (200 mL). The combined organic layers were washed with brine (100 mL×2) and dried over anhydrous sodium sulfate. The solution was filtered and concentrated to afford the title compound (12 g, 91% yield). MS (ESI⁺) m/z 338.7 (M+Na)⁺.

### Example 1-24

### tert-butyl [(1R)-1-(4-bromophenyl)-2-{[tert-butyl(dimethyl)silyl]oxy}ethyl]carbamate

Tert-butyldimethylchlorosilane (7.15 g, 47.4 mmol) was added to a solution of Example 1-23 (12 g, 38 mmol) in N,N-dimethylformamide (180 mL) at 20° C., the, imidazole (6.46 g, 95 mmol) was added. The reaction mixture was stirred at ambient temperature for 8 hours, quenched with saturated aqueous NH₄Cl (300 mL), and extracted with ethyl acetate (300 mL). The combined organic phases were washed with brine (150 mL×2), dried over anhydrous sodium sulfate, and concentrated in vacuo. The residue was purified by flash column chromatography (silica gel, petroleum ether/ethyl acetate=50:1) to give the title compound (15 g, 87% yield). ¹H NMR (400 MHz, DMSO-d₆) δ ppm 7.56 (d, J=8.4 Hz, 2H), 7.33 (d, J=8.4 Hz, 2H), 4.63-4.60 (m, 1H), 3.70-3.65 (m, 2H), 1.42 (s, 9H), 0.86 (s, 9H), 0.01 (d, J=9.2 Hz, 6H); MS (ESI) m/z 452.7 (M+Na)⁺.

### Example 1-25

### tert-butyl {(1R)-2-hydroxy-1-[4-(4-methyl-1,3-thiazol-5-yl)phenyl]ethyl}carbamate

Potassium carbonate (4.82 g, 34.8 mmol), palladium(II) acetate (0.209 g, 0.929 mmol), tricyclohexylphosphonium tetrafluoroborate (0.684 g, 1.86 mmol) and pivalic acid (0.712 g, 6.97 mmol) were placed in a screw-cap vial equipped with a magnetic stir bar. N,N-Dimethylformamide (77.5 mL) was added. Example 1-24 (10.0 g, 23.2 mmol) and 4-methylthiazole (2.75 mL, 30.2 mmol) were added. The mixture was bubbled with nitrogen for 3 minutes. The reaction mixture was then vigorously stirred at 100° C. for 16 hours. The solution was then cooled to ambient temperature, diluted with ethyl acetate (150 mL×2), washed with brine (100 mL×3), dried over Na₂SO₄, filtered, and concentrated. The crude product was dissolved in tetrahydrofuran (100 mL), and then 1 M tetrabutylammonium fluoride in tetrahydrofuran (25 mL) was added. The mixture was stirred for 3 hours, diluted with ethyl acetate (150 mL×2), washed with saturated ammonium chloride (100 mL×5), dried over anhydrous sodium sulfate, filtered, and concentrated. This material was purified by flash column chromatography (silica gel, ethyl acetate/petroleum ether- 1/10) to afford the title compound (7.0 g, 86% yield). ¹H NMR (400 MHz, DMSO-d₆) δ ppm 8.99 (s, 1H), 7.44 (d, J=8.0 Hz, 2H), 7.38 (d, J=8.4 Hz, 2H), 7.29 (d, J=8.4 Hz, 1H), 4.83 (t, J=5.6 Hz, 1H), 4.57-4.55 (m, 1H), 3.51 (t, J=6.4 Hz, 2H), 2.46 (s, 3H), 1.37 (s, 9H); MS(ESI) m/z 335.7 (M+H)⁺.

### Example 1-26

### (2R)-2-amino-2-[4-(4-methyl-1,3-thiazol-5-yl)phenyl]ethan-1-ol hydrochloride

A solution of Example 1-25 (6.0 g, 18 mmol) in 1,4-dioxane (120 mL) and 4 N HCl/dioxane (40 mL) was stirred at 20° C. for 16 hours. The volatiles were removed under reduced pressure to obtain the title compound (4.8 g, 94% yield). MS(ESI) m/z 235.7 (M+H)⁺.

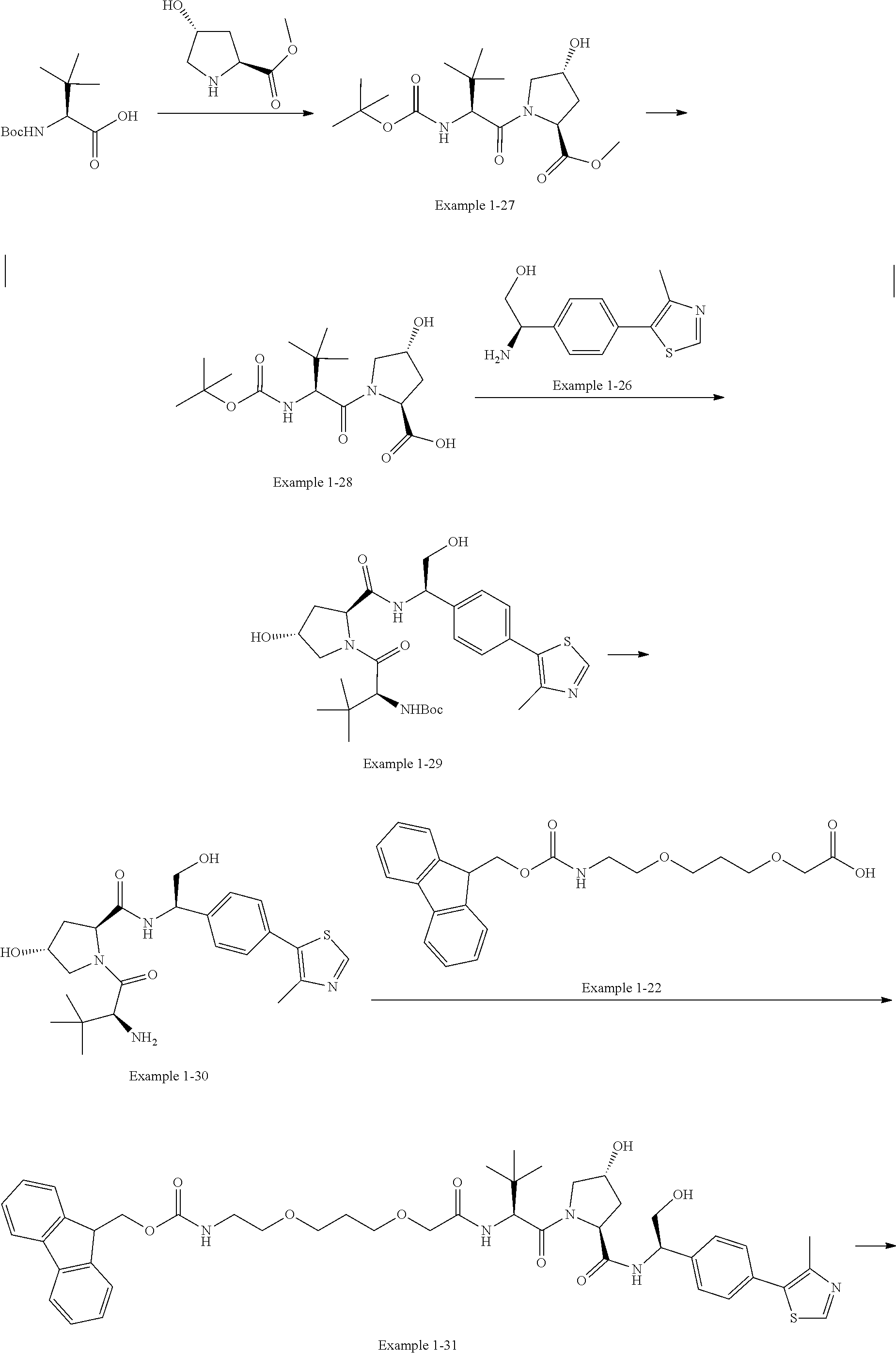

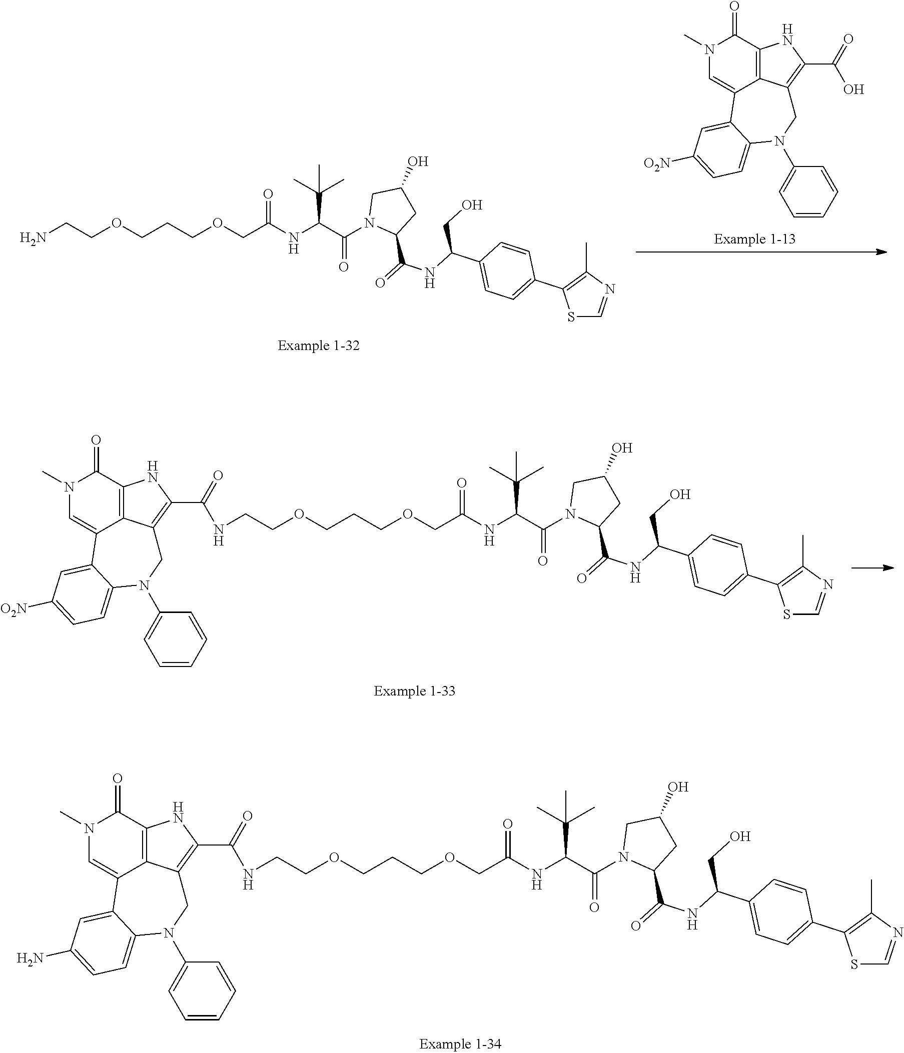 [SMILES: Cc1ncsc1-c1ccc([C@H](CO)NC(=O)[C@@H]2C[C@@H](O)CN2C(=O)[C@@H](NC(=O)COCCCOCCN)C(C)(C)C)cc1 ; Cn1cc2c3c(c(C(=O)O)[nH]c3c1=O)CN(c1ccccc1)c1ccc([N+](=O)[O-])cc1-2 ; Cc1ncsc1-c1ccc([C@H](CO)NC(=O)[C@@H]2C[C@@H](O)CN2C(=O)[C@@H](NC(=O)COCCCOCCNC(=O)c2[nH]c3c(=O)n(C)cc4c3c2CN(c2ccccc2)c2ccc([N+](=O)[O-])cc2-4)C(C)(C)C)cc1 ; Cc1ncsc1-c1ccc([C@H](CO)NC(=O)[C@@H]2C[C@@H](O)CN2C(=O)[C@@H](NC(=O)COCCCOCCNC(=O)c2[nH]c3c(=O)n(C)cc4c3c2CN(c2ccccc2)c2ccc(N)cc2-4)C(C)(C)C)cc1]

### Example 1-27

### methyl N-(tert-butoxycarbonyl)-3-methyl-L-valyl-(4R)-4-hydroxy-L-prolinate

1-((Dimethylamino)(dimethyliminio)methyl)-1H-[1,2,3]triazolo[4,5-b]pyridine 3-oxide hexafluorophosphate(V) (41.6 g, 109 mmol, HATU) was added to a mixture of N-(tert-butoxycarbonyl)-3-methyl-L-valine (23 g, 99 mmol), methyl (4R)-4-hydroxy-L-prolinate hydrochloride (18.06 g, 99 mmol) and N,N-diisopropylethylamine (60.8 mL, 348 mmol) in N,N-dimethylformamide (230 mL) at 0° C. The mixture was stirred at 20° C. for 16 hours. The reaction mixture was quenched with water (500 mL) and extracted with ethyl acetate (1000 mL). The combined organic layers were washed with 5% citric acid (300 mL×3), saturated NaHCO₃ solution (300 mL×2) and brine (300 mL×4), and dried over anhydrous sodium sulfate. The organic solution was filtered and concentrated to afford the title compound (35 g, 93% yield). ¹H NMR (400 MHz, DMSO-d₆) δ ppm 6.58 (d, J=9.2 Hz, 1H), 5.28 (d, J=3.6 Hz, 1H), 4.48-4.34 (m, 2H), 4.22 (d, J=9.2 Hz, 1H), 3.79-3.59 (m, 5H), 2.22-2.10 (m, 1H), 2.02-1.88 (m, 1H), 1.44 (s, 9H), 0.99 (s, 9H); MS(ESI) m/z 381.7 (M+Na)⁺.

### Example 1-28

### N-(tert-butoxycarbonyl)-3-methyl-L-valyl-(4R)-4-hydroxy-L-proline

A mixture of Example 1-27 (10 g, 28 mmol) and lithium hydroxide hydrate (5.85 g, 139 mmol) in tetrahydrofuran (100 mL) and water (50 mL) was stirred at 20° C. for 4 hours. The mixture was neutralized with 1 N hydrochloric acid to pH=4. The resulting suspension was filtered, and washed with water (15 mL), filtered, and dried under house vacuum at ambient temperature to give the title compound (8.8 g, 87% yield). MS (ESI) m/z 367.1 (M+Na)⁺.

### Example 1-29

### N-(tert-butoxycarbonyl)-3-methyl-L-valyl-(4R)-4-hydroxy-N-{(1R)-2-hydroxy-1-[4-(4-methyl-1,3-thiazol-5-yl)phenyl]ethyl}-L-prolinamide

1-((Dimethylamino)(dimethyliminio)methyl)-1H-[1,2,3]triazolo[4,5-b]pyridine 3-oxide hexafluorophosphate(V) (6.68 g, 17.6 mmol, HATU) was added to a mixture of Example 1-28 (5.5 g, 16 mmol), Example 1-26 (4.32 g, 16.0 mmol) and N,N-diisopropylethylamine (9.76 mL, 55.9 mmol) in N,N-dimethylformamide (60 mL) at 0° C. The mixture was stirred at 20° C. for 5 hours. The reaction mixture was diluted with water (200 mL) and then extracted with ethyl acetate (200 mL×3). The combined organic fractions were washed with brine (100 mL×3), dried over anhydrous sodium sulfate, filtered, and concentrated in vacuo. The residue was purified by sequential silica gel column chromatography (dichloromethane/methanol=20/1) and reverse phase chromatography (RP-C18 cartridge, 40-60 μm, 60 Å) (H₂O(0.01% NH₄HCO₃)(A)/methanol(B), gradient from 5-60% of B at 0 minute-35 minutes, then 60-80% of B at 35 minutes-56 minutes, to give the title compound (6.94 g, 76% yield). ¹H NMR (400 MHz, CDCl₃) δ ppm 8.68 (s, 1H), 7.52 (d, J=8.4 Hz, 1H), 7.43 (s, 4H), 5.23 (d, J=9.2 Hz, 1H), 5.21-5.16 (m, 1H), 4.64 (t, J=8.2 Hz, 1H), 4.49 (brs, 1H), 4.22 (d, J=9.2 Hz, 1H), 4.07 (d, J=11.6 Hz, 1H), 3.99-3.91 (m, 1H), 3.89-3.81 (m, 1H), 3.74-3.67 (m, 1H), 3.65-3.58 (m, 1H), 3.50-3.47 (m, 1H), 2.52 (s, 3H), 2.34-2.24 (m, 1H), 2.20-2.10 (m, 1H), 1.41 (s, 9H), 1.04 (s, 9H); MS(ESI) m/z 560.8 (M+H)⁺.

### Example 1-30

### 3-methyl-L-valyl-(4R)-4-hydroxy-N-{(1R)-2-hydroxy-1-[4-(4-methyl-1,3-thiazol-5-yl)phenyl]ethyl}-L-prolinamide trifluoroacetate

A round bottom flask with stir bar was charged with Example 1-29 (176 mg, 0.314 mmol), 2,2,2-trifluoroacetic acid (1.0 mL, 13 mmol) and dichloromethane (4.0 mL). The solution was stirred at ambient temperature for 90 minutes. The reaction mixture was concentrated in vacuo, chased with three portions of toluene, then vacuum dried overnight. The title compound was carried forward without purification assuming a quantitative yield. ¹H NMR (501 MHz, DMSO-d₆) δ ppm 8.98 (s, 1H), 7.45-7.32 (m, 2H), 7.30-7.09 (m, 2H), 5.24 (dtd, J=13.1, 7.5, 5.4 Hz, 1H), 4.69-4.52 (m, 1H), 4.02 (q, J=5.6 Hz, 1H), 3.91 (q, J=5.5 Hz, 1H), 3.81 (ddd, J=12.7, 10.9, 3.9 Hz, 1H), 3.69-3.54 (m, 1H), 3.54-3.46 (m, 1H), 2.44 (d, J=2.3 Hz, 2H), 2.28 (s, 3H), 2.17-2.03 (m, 1H), 1.01 (s, 9H); MS (ESI⁺) m/z 461.4 (M+H)⁺.

### Example 1-31

### N-[1-(9H-fluoren-9-yl)-3,13-dioxo-2,7,11-trioxa-4-azatridecan-13-yl]-3-methyl-L-valyl-(4R)-4-hydroxy-N-{(1R)-2-hydroxy-1-[4-(4-methyl-1,3-thiazol-5-yl)phenyl]ethyl}-L-prolinamide

A flask with stir bar was charged with Example 1-22 (0.759 g, 1.90 mmol) and 2-(3H-[1,2,3]triazolo[4,5-b]pyridin-3-yl)-1,1,3,3-tetramethylisouronium hexafluorophosphate(V) (0.928 g, 2.44 mmol) in N,N-dimethylformamide (9.39 mL) to give a colorless solution. Example 1-30 (1.002 g, 1.878 mmol) was added as a solution in N,N-dimethylformamide (9.39 mL). N-Ethyl-N-isopropylpropan-2-amine (2.60 mL, 15.0 mmol) was added. The reaction mixture was stirred at ambient temperature for 1.5 hours. The reaction mixture was partitioned between ethyl acetate (200 mL) and brine (100 mL). The aqueous layer was extracted with additional ethyl acetate (200 mL). The combined organic layers were washed with brine, dried over anhydrous sodium sulfate, filtered, and concentrated. The residue was chromatographed on a 40 g Teledyne ISCO RediSep Rf Gold® silica cartridge eluted with 10-100% 3:1 ethyl acetate:ethanol/heptanes to provide the title compound (1.422 g, 90%). ¹H NMR (400 MHz, DMSO-d₆) δ ppm 8.94 (s, 1H), 8.38 (d, J=8.0 Hz, 1H), 7.85 (dd, J=7.6, 1.0 Hz, 2H), 7.66 (d, J=7.4 Hz, 2H), 7.44-7.32 (m, 4H), 7.29 (td, J=7.5, 3.0 Hz, 4H), 5.10 (d, J=3.5 Hz, 1H), 4.82 (dt, J=8.3, 6.2 Hz, 1H), 4.74 (dd, J=6.4, 5.1 Hz, 1H), 4.55-4.40 (m, 2H), 4.40-4.23 (m, 4H), 3.92-3.82 (m, 2H), 3.57 (q, J=5.1, 4.1 Hz, 2H), 3.57-3.46 (m, 2H), 3.46-3.31 (m, 3H), 3.11 (q, J=5.7 Hz, 2H), 2.42 (s, 3H), 2.05 (s, 2H), 2.11-1.98 (m, 1H), 1.76 (tt, J=12.7, 5.6 Hz, 2H), 0.91 (s, 9H); MS (ESI⁺) m/z 842.6 (M+H)⁺.

### Example 1-32

### N-{[3-(2-aminoethoxy)propoxy]acetyl}-3-methyl-L-valyl-(4R)-4-hydroxy-N-{(1R)-2-hydroxy-1-[4-(4-methyl-1,3-thiazol-5-yl)phenyl]ethyl}-L-prolinamide

A 100 mL flask with stir bar was charged with a solution of Example 1-31 (1.42 g, 1.686 mmol) in N,N-dimethylformamide (17 mL) was treated with diethylamine (0.26 mL, 2.510 mmol). The reaction mixture was stirred at ambient temperature overnight. The volatiles were removed under reduced pressure, and the residue was loaded onto a 20 g Teledyne ISCO RediSep Rf Gold® silica cartridge and eluted with a gradient of 0-20% ammonia saturated methanol/dichloromethane to provide the title compound (0.683 g, 65%). ¹H NMR (400 MHz, DMSO-d₆) δ ppm 8.95 (s, 1H), 8.40 (d, J=8.0 Hz, 1H), 7.45-7.32 (m, 2H), 4.87-4.77 (m, 1H), 4.54-4.42 (m, 1H), 4.06 (m, 1H), 3.95-3.82 (m, 1H), 3.64-3.47 (m, 2H), 3.44 (t, J=6.4 Hz, 1H), 3.32 (t, J=5.7 Hz, 1H), 3.40-3.16 (m, 13H), 2.64 (t, J=5.7 Hz, 1H), 2.42 (s, 1H), 2.03 (ddd, J=10.2, 7.8, 2.2 Hz, 1H), 1.81-1.70 (m, 1H), 0.91 (s, 9H); MS (ESI⁺) m/z 620.4 (M+H)⁺.

### Example 1-33

### 3-methyl-N-[(3-{2-[(2-methyl-10-nitro-3-oxo-7-phenyl-3,4,6,7-tetrahydro-2H-2,4,7-triazadibenzo[cd, f]azulene-5-carbonyl)amino]ethoxy}propoxy)acetyl]-L-valyl-(4R)-4-hydroxy-N-{(1R)-2-hydroxy-1-[4-(4-methyl-1,3-thiazol-5-yl)phenyl]ethyl}-L-prolinamide

A mixture of Example 1-13 (153 mg, 0.367 mmol), Example 1-32 (263 mg, 0.424 mmol), ((3H-[1,2,3]triazolo[4,5-b]pyridin-3-yl)oxy)tri(pyrrolidin-1-yl)phosphonium hexafluorophosphate(V) (231 mg, 0.443 mmol) and N-ethyl-N-isopropylpropan-2-amine (0.250 μL, 1.43 μmol) in N,N-dimethylformamide (3.6 mL) was stirred at ambient temperature for 90 minutes. The reaction mixture was diluted with ethyl acetate (100 mL), washed with brine (2×100 mL), dried over anhydrous sodium sulfate, filtered and concentrated to provide the title compound (0.369 g, 99% yield). ¹H NMR (400 MHz, DMSO-d₆) δ ppm 8.94 (s, 1H), 8.70 (d, J=2.7 Hz, 1H), 8.43 (m, 1H), 8.36 (d, J=8 Hz, 1H), 8.16 (dd, J=8.6, 2.7 Hz, 1H), 7.93 (m, 1H), 7.54 (d, J=8.6 Hz, 1H), 7.43-7.27 (m, 4H), 7.02-6.94 (m, 2H), 6.55 (t, J=7.3 Hz, 1H), 6.49-6.42 (m, 2H), 5.09 (d, J=3.5 Hz, 2H), 4.82 (q, J=6.6 Hz, 2H), 4.73 (dd, J=6.4, 5.1 Hz, 2H), 4.54-4.35 (m, 3H), 4.25 (s, 2H), 3.90-3.81 (m, 3H), 3.61-3.45 (m, 10H), 2.42 (s, 3H), 2.08-1.98 (m, 1H), 1.81 (d, J=6.4 Hz, 2H), 0.90 (s, 9H); MS (ESI⁺) m/z 1018.2 (M+H)⁺.

### Example 1-34

### N-[(3-{2-1[(10-amino-2-methyl-3-oxo-7-phenyl-3,4,6,7-tetrahydro-2H-2,4,7-triazadibenzo[cd, f]azulene-5-carbonyl)amino]ethoxy}propoxy)acetyl]-3-methyl-L-valyl-(4R)-4-hydroxy-N-{(1R)-2-hydroxy-1-[4-(4-methyl-1,3-thiazol-5-yl)phenyl]ethyl}-L-prolinamide

A mixture Example 1-33 (369 mg, 0.362 mmol), ammonium chloride (107 mg, 2.00 mmol), and iron powder, <10 micron (292 mg, 5.23 mmol) in tetrahydrofuran (8.00 mL), ethanol (8.00 mL), and water (1.6 mL) was heated at 90° C. for 150 minutes. After cooling to ambient temperature, the reaction mixture was filtered through a pad of diatomaceous earth, and the filter pad was washed with 5% methanol/dichloromethane. The filtrate was concentrated, and the residues chromatographed on a 4 g silica cartridge eluted with 0-10% ammonia saturated methanol/dichloromethane to provide the title compound (218 mg, 61%). ¹H NMR (501 MHz, DMSO-d₆) δ ppm 12.13 (s, 1H), 8.95 (s, 1H), 8.40 (m, 2H), 7.48 (s, 1H), 7.38 (m, 4H), 7.32 (m, 1H), 6.96 (m, 2H), 6.87 (dd, J=8.5, 7.0 Hz, 2H), 6.60 (dd, J=8.3, 2.5 Hz, 1H), 6.39 (t, J=7.2 Hz, 1H), 6.33 (d, J=8.2 Hz, 2H), 5.84 (d, J=16.9 Hz, 1H), 5.16 (s, 2H), 5.12 (dd, J=3.6, 1.3 Hz, 1H), 4.83 (q, J=6.6 Hz, 1H), 4.76 (t, J=5.8 Hz, 1H), 4.52 (d, J=9.5 Hz, 1H), 4.48 (t, J=8.2 Hz, 1H), 4.31 (d, J=2.5 Hz, 1H), 4.28 (m, 2H), 3.89 (m, 2H), 3.62-3.54 (m, 4H), 3.44 (m, 1H), 3.32 (s, 3H), 2.43 (s, 3H), 2.05 (m, 1H), 1.82 (t, J=6.4 Hz, 1H), 1.76 (m, 1H), 0.92 (s, 9H); MS (ESI⁺) m/z 988.4 (M+H)⁺.

### Example 2. LD1

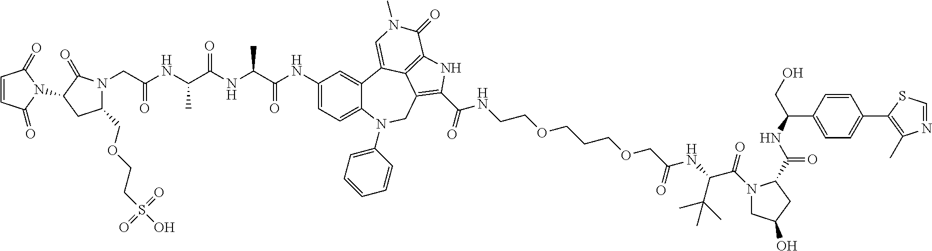 [SMILES: Cc1ncsc1-c1ccc([C@H](CO)NC(=O)[C@@H]2C[C@@H](O)CN2C(=O)[C@@H](NC(=O)COCCCOCCNC(=O)c2[nH]c3c(=O)n(C)cc4c3c2CN(c2ccccc2)c2ccc(NC(=O)[C@H](C)NC(=O)[C@H](C)NC(=O)CN3C(=O)[C@@H](N5C(=O)C=CC5=O)C[C@H]3COCCS(=O)(=O)O)cc2-4)C(C)(C)C)cc1]

### N-[(3-{2-[(10-{I[N-({(3S,5S)-3-(2,5-dioxo-2,5-dihydro-1H-pyrrol-1-yl)-2-oxo-5-[(2-sulfoethoxy)methyl]pyrrolidin-1-yl}acetyl)-L-alanyl-L-alanyl]amino}-2-methyl-3-oxo-7-phenyl-3,4,6,7-tetrahydro-2H-2,4,7-triazadibenzo[cd, f]azulene-5-carbonyl)amino]ethoxyIpropoxy)acetyl]-3-methyl-L-valyl-(4R)-4-hydroxy-N-{(1R)-2-hydroxy-1-[4-(4-methyl-1,3-thiazol-5-yl)phenyl]ethyl}-L-prolinamide

### (Formula (III), LD1)

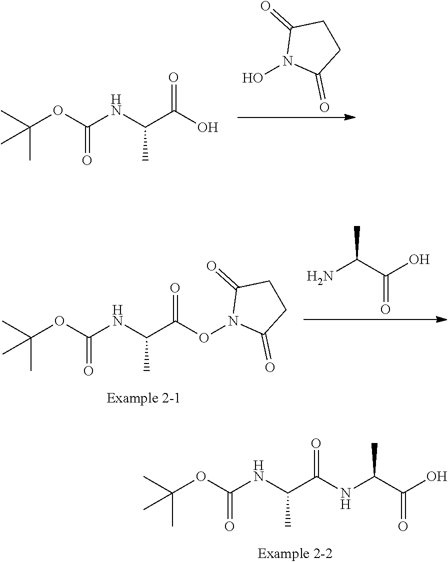

### Example 2-1

### 2,5-dioxopyrrolidin-1-yl N-(tert-butoxycarbonyl)-L-alaninate

A mixture of 1-hydroxypyrrolidine-2,5-dione (33.5 g, 291 mmol) and N-(tert-butoxycarbonyl)-L-alanine (50.0 g, 264 mmol) in dichloromethane (500 mL) was cooled to 0° C. N,N′-dicyclohexylcarbodiimide (60.0 g, 291 mmol) was added to the solution. The reaction mixture was stirred at 25° C. for about 16 hours, filtered, and washed with additional dichloromethane (3×100 mL). The filtrate was concentrated under reduced pressure to give the title compound (54 g, 71% yield). ¹H NMR (400 MHz, DMSO-d₆) δ ppm 7.60 (d, J=7.3 Hz, 1H), 4.39 (p, J=7.3 Hz, 1H), 2.78 (s, 4H), 1.37 (s, 12H).

### Example 2-2

### N-(tert-butoxycarbonyl)-L-alanyl-L-alanine

L-alanine (6.22 g, 69.9 mmol) and sodium bicarbonate (11.74 g, 140 mmol) in water (150 mL) were added to a solution of Example 2-1 (20.0 g, 69.9 mmol) in 1,2-dimethoxyethane (150 mL). The reaction mixture was stirred at 25° C. for about 16 hours. The volatile solvent was removed under reduced pressure. The pH of the remaining solution was adjusted to 1 with aqueous citric acid. The precipitate was collected by filtration, washed with additional water, and dried to give the title compound (16 g, 88% yield). ¹H NMR (400 MHz, DMSO-d₆) δ ppm 7.96 (d, J=7.3 Hz, 1H), 6.83 (d, J=7.5 Hz, 1H), 4.20 (p, J=7.2 Hz, 1H), 4.09-3.89 (m, 1H), 1.37 (s, 9H), 1.27 (d, J=7.3 Hz, 3H), 1.17 (d, J=7.1 Hz, 3H).

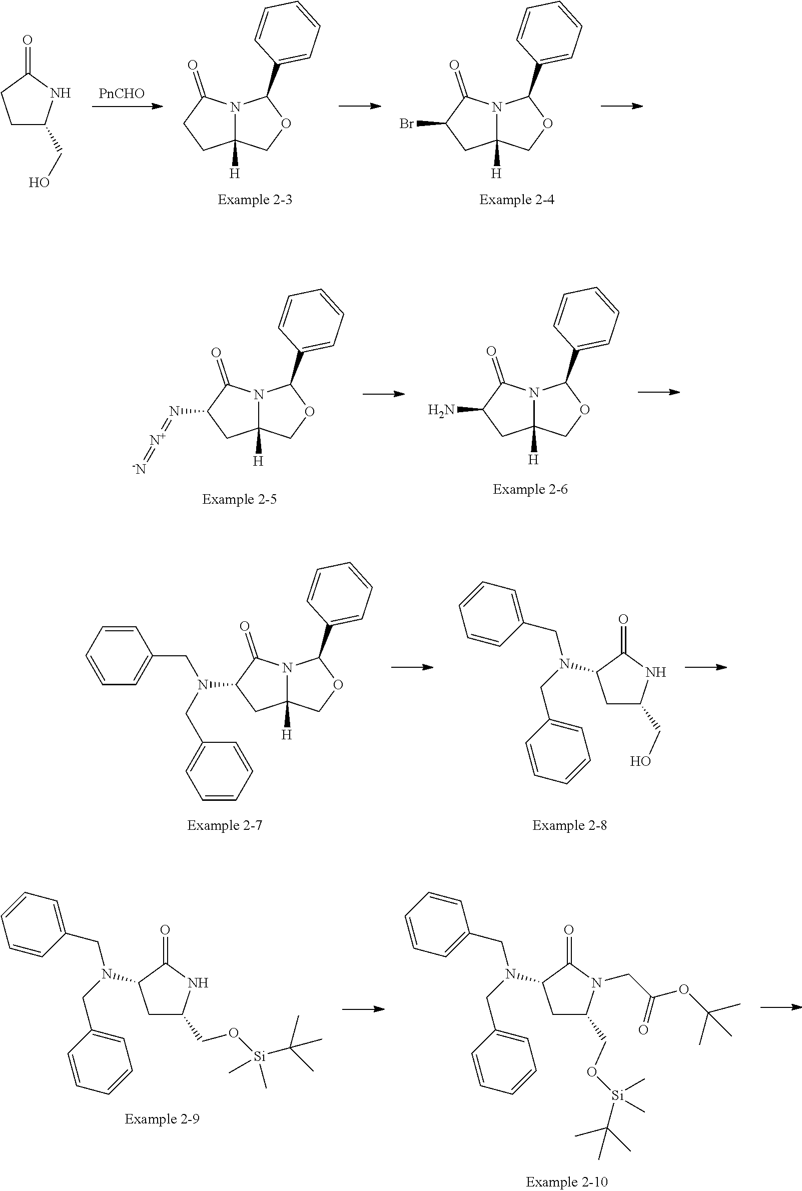

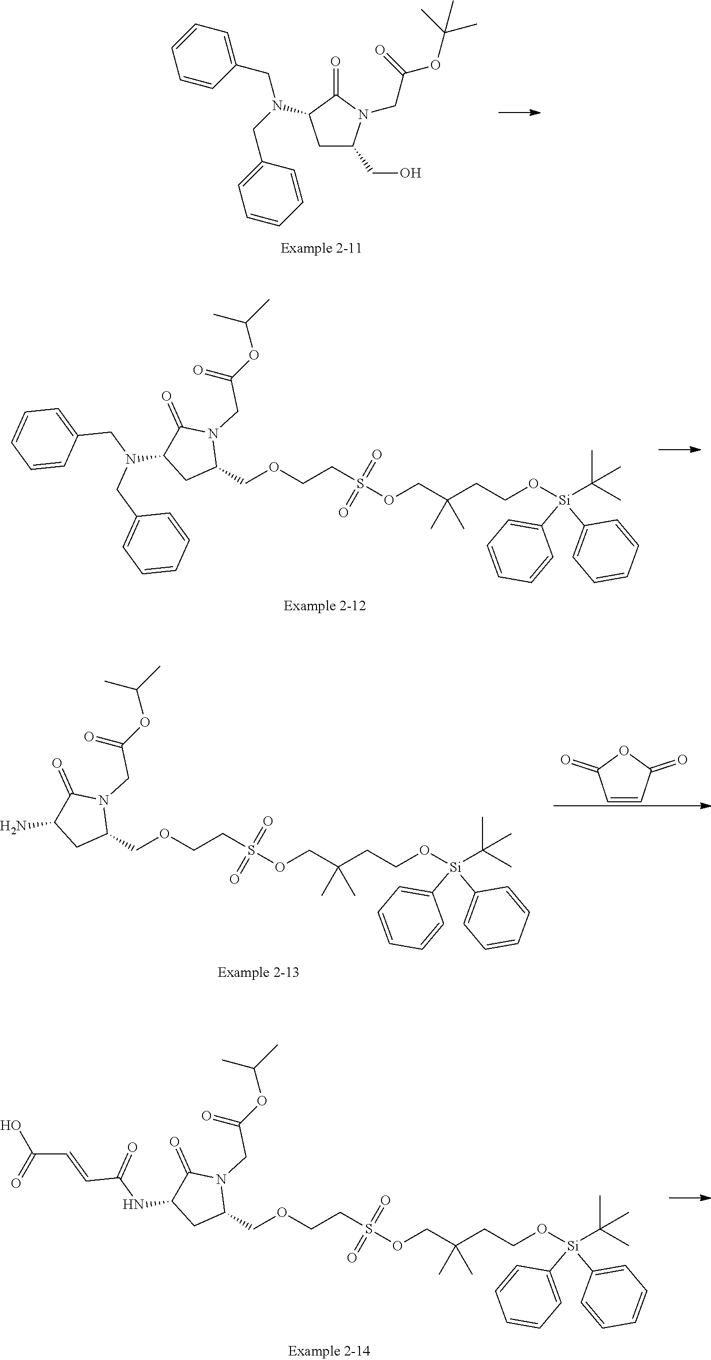 [SMILES: CC(C)(C)OC(=O)CN1C(=O)[C@@H](N(Cc2ccccc2)Cc2ccccc2)C[C@H]1CO ; CC(C)OC(=O)CN1C(=O)[C@@H](N(Cc2ccccc2)Cc2ccccc2)C[C@H]1COCCS(=O)(=O)OCC(C)(C)CCO[Si](c1ccccc1)(c1ccccc1)C(C)(C)C ; CC(C)OC(=O)CN1C(=O)[C@@H](N)C[C@H]1COCCS(=O)(=O)OCC(C)(C)CCO[Si](c1ccccc1)(c1ccccc1)C(C)(C)C ; O=C1C=CC(=O)O1 ; CC(C)OC(=O)CN1C(=O)[C@@H](NC(=O)/C=C/C(=O)O)C[C@H]1COCCS(=O)(=O)OCC(C)(C)CCO[Si](c1ccccc1)(c1ccccc1)C(C)(C)C]

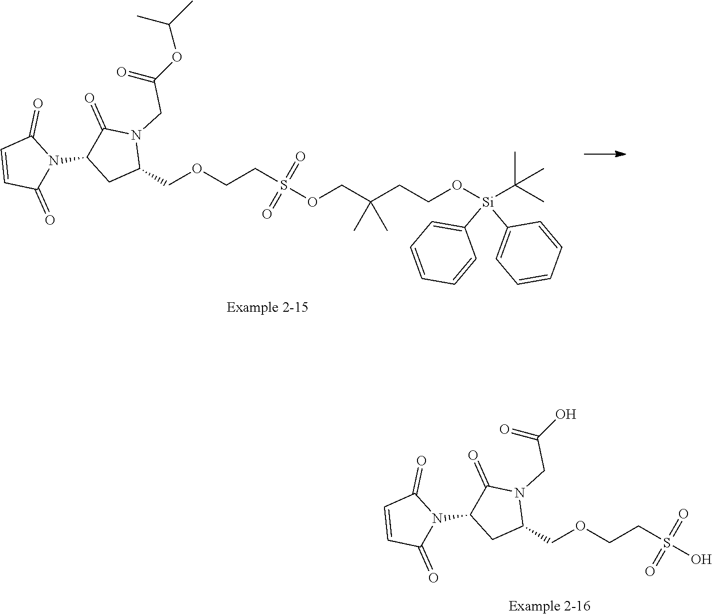 [SMILES: CC(C)OC(=O)CN1C(=O)[C@@H](N2C(=O)C=CC2=O)C[C@H]1COCCS(=O)(=O)OCC(C)(C)CCO[Si](c1ccccc1)(c1ccccc1)C(C)(C)C ; O=C(O)CN1C(=O)[C@@H](N2C(=O)C=CC2=O)C[C@H]1COCCS(=O)(=O)O]

### Example 2-3

### (3R,7aS)-3-phenyltetrahydro-3H,5H-pyrrolo[1,2-c][11,3]oxazol-5-one

A mixture of (S)-5-(hydroxymethyl)pyrrolidin-2-one (25 g, 0.22 mmol), benzaldehyde (25.5 g, 0.24 mmol) and p-toluenesulfonic acid monohydrate (0.50 g, 0.0026 mmol) in toluene (300 mL) was heated under reflux using a Dean-Stark trap under a drying tube for 16 hours. The reaction mixture was cooled to ambient temperature, and the solvent was decanted from the insoluble materials. The solvent mixture was washed with saturated aqueous sodium bicarbonate mixture and saturated aqueous sodium chloride. The organic layer was dried over anhydrous sodium sulfate, filtered and concentrated under reduced pressure. The residue was purified by flash chromatography (silica gel, 35/65 heptane/ethyl acetate) to give the title compound (35.3 g, 80% yield). ¹H NMR (400 MHz, CDCl₃) δ ppm 7.47 (dd, J=8.0, 1.7 Hz, 2H), 7.43-7.34 (m, 2H), 7.38-7.26 (m, 1H), 6.36 (s, 1H), 4.25 (dd, J=8.0, 6.3 Hz, 1H), 4.17 (ddd, J=13.8, 7.7, 5.7 Hz, 1H), 3.51 (t, J=8.0 Hz, 1H), 2.83 (ddd, J=17.4, 10.1, 9.0 Hz, 1H), 2.57 (ddd, J=17.4, 10.0, 3.8 Hz, 1H), 2.40 (dddd, J=13.7, 10.3, 7.5, 3.8 Hz, 1H), 1.96 (dtd, J=13.5, 9.5, 5.4 Hz, 1H); MS (DCI) m/z 204.0 (M+H)⁺.

### Example 2-4

### (3R,6R,7aS)-6-bromo-3-phenyltetrahydro-3H,5H-pyrrolo[1,2-c][1,3]oxazol-5-one

To a mixture of Example 2-3 (44.6 g, 0.22 mmol) in tetrahydrofuran (670 mL) cooled to −77° C. under nitrogen was added lithium bis(trimethylsilyl)amide (1.0 M in hexanes, 250 mL) dropwise over 40 minutes, keeping the internal temperature less than −73° C. The reaction mixture was stirred at −77° C. for 2 hours, and then bromine (12.5 mL, 0.24 mmol) was added dropwise over 20 minutes, keeping the internal temperature less than −64° C. The reaction mixture was stirred at −77° C. for 75 minutes and was then quenched by the addition of cold 10% aqueous sodium thiosulfate (150 mL) to the −77° C. reaction mixture. The reaction mixture was warmed to ambient temperature and partitioned between half-saturated aqueous ammonium chloride mixture and ethyl acetate. The layers were separated, and the organic layer was washed with water and saturated aqueous sodium chloride, dried over anhydrous sodium sulfate, filtered and concentrated under reduced pressure. The residue was purified by flash chromatography (silica gel, eluted with a gradient of 80/20, 75/25, and 70/30 heptane/ethyl acetate) to give the title compound (19.3 g, 31.2%). ¹H NMR (400 MHz, CDCl₃) δ ppm 7.50-7.32 (m, 5H), 6.33 (s, 1H), 4.53 (dd, J=6.9, 1.3 Hz, 1H), 4.48-4.37 (m, 1H), 4.30 (ddd, J=8.4, 6.4, 0.5 Hz, 1H), 3.63 (dd, J=8.4, 7.2 Hz, 1H), 2.67 (ddd, J=14.6, 6.2, 1.3 Hz, 1H), 2.50 (dt, J=14.7, 6.9 Hz, 1H); MS (DCI) m/z 299.0 and 301.0 (M+NH₃+H)⁺.

### Example 2-5

### (3R,6S,7aS)-6-azido-3-phenyltetrahydro-3H,5H-pyrrolo[1,2-c][1,3]oxazol-5-one

To a mixture of Example 2-4 (19.3 g, 0.068 mmol) in N,N-dimethylformamide (100 mL) was added sodium azide (13.5 g, 0.208 mmol). The reaction mixture was heated to 60° C. for 2.5 hours. The reaction mixture was cooled to ambient temperature and partitioned between water (500 mL) and ethyl acetate (200 mL). The layers were separated, and the organic layer was washed with saturated aqueous sodium chloride. The combined aqueous layers were back extracted with ethyl acetate (50 mL). The combined organic layers were dried with anhydrous sodium sulfate, filtered, and concentrated under reduced pressure. The residue was purified by silica gel chromatography, eluted with 78/22 heptane/ethyl acetate, to give the title compound (13.5 g, 81%). ¹H NMR (400 MHz, CDCl₃) δ ppm 7.50-7.41 (m, 2H), 7.45-7.32 (m, 3H), 6.38 (s, 1H), 4.56 (dd, J=10.5, 8.5 Hz, 1H), 4.33 (dd, J=8.4, 6.2 Hz, 1H), 4.06 (ddd, J=13.9, 7.5, 6.4 Hz, 1H), 3.62 (t, J=8.0 Hz, 1H), 2.78 (ddd, J=13.0, 8.5, 6.6 Hz, 1H), 1.81 (ddd, J=13.0, 10.4, 7.4 Hz, 1H); MS (DCI) m/z 262.0 (M+NH₃+H)⁺.

### Example 2-6

### (3R,6S,7aS)-6-amino-3-phenyltetrahydro-3H,5H-pyrrolo[1,2-c][1,3]oxazol-5-one

To a mixture of Example 2-5 (13.5 g, 0.553 mmol) in tetrahydrofuran (500 mL) and water (50 mL) was added polymer-supported triphenylphosphine (55 g, 3 mmol PPh₃/g, Aldrich #366455). The reaction mixture was mechanically stirred overnight at ambient temperature. The reaction mixture was filtered through diatomaceous earth, eluting sequentially with ethyl acetate and toluene. The filtrate was concentrated under reduced pressure. The residue was dissolved in dichloromethane (100 mL), dried over anhydrous sodium sulfate, and filtered. The filtrate was concentrated to give the title compound which was used in the subsequent step without further purification (11.3 g, 94% yield). ¹H NMR (400 MHz, CDCl₃) δ ppm 7.52-7.21 (m, 5H), 6.34 (s, 1H), 4.28 (dd, J=8.4, 6.3 Hz, 1H), 4.10-3.93 (m, 2H), 3.59 (dd, J=8.4, 7.2 Hz, 1H), 2.88-2.77 (m, 1H), 1.69-1.56 (m, 1H); MS (DCI) m/z 219.0 (M+H)⁺.

### Example 2-7

### (3R,6S,7aS)-6-(dibenzylamino)-3-phenyltetrahydro-3H,5H-pyrrolo[1,2-c][1,3]oxazol-5-one

To a mixture of Example 2-6 (11.3 g, 0.0518 mmol) in N,N-dimethylformamide (100 mL) was added potassium carbonate (7.0 g, 0.051 mmol), potassium iodide (4.2 g, 0.025 mmol), and benzyl bromide (14.5 mL, 0.122 mmol). The reaction mixture was stirred at ambient temperature overnight and then partitioned between water and ethyl acetate. The layers were separated, and the organic layer was washed with saturated aqueous sodium chloride. The combined aqueous layers were back extracted with ethyl acetate. The combined organic layers were dried over anhydrous sodium sulfate, filtered and concentrated under reduced pressure. The residue was purified by flash chromatography (silica gel, eluted with a gradient of 10% to 15% ethyl acetate in heptane) then titrated with heptane and air dried to give the title compound (14.3 g, 69.3% yield). ¹H NMR (400 MHz, CDCl₃) δ ppm 7.50-7.39 (m, 5H), 7.43-7.22 (m, 9H), 4.29 (dd, J=8.3, 6.2 Hz, 1H), 4.11 (dd, J=11.3, 8.7 Hz, 1H), 4.07 (s, 1H), 4.04 (s, 1H), 3.92 (qd, J=7.4, 6.2 Hz, 1H), 3.74 (d, J=13.9 Hz, 2H), 3.53 (t, J=8.0 Hz, 1H), 2.50 (ddd, J=12.7, 8.6, 7.0 Hz, 1H), 1.92 (ddd, J=12.8, 11.3, 7.5 Hz, 1H); MS (DCI) m/z 399.1 (M+H)⁺.

### Example 2-8

### (3S,5S)-3-(dibenzylamino)-5-(hydroxymethyl)pyrrolidin-2-one

To a mixture of Example 2-7 (13 g, 0.033 mmol) in tetrahydrofuran (130 mL) was added para-toluene sulfonic acid monohydrate (12.4 g, 0.0652 mmol) and water (50 mL), and the reaction mixture was heated to 65° C. for 6 days. The reaction mixture was cooled to ambient temperature and quenched by the addition of saturated aqueous sodium bicarbonate and ethyl acetate. The layers were separated, and the organic layer was washed with saturated aqueous sodium chloride. The combined aqueous layers were back extracted with ethyl acetate. The combined organic layers were dried over anhydrous sodium sulfate, filtered, and concentrated under reduced pressure. The residue was triturated with heptane (150 mL) and air dried to give the title compound (9.3 g, 92% yield). ¹H NMR (400 MHz, CDCl₃) δ ppm 7.43 (d, J=8.2 Hz, 4H), 7.31 (t, J=7.8 Hz, 4H), 7.27-7.19 (m, 2H), 7.10 (s, 1H), 3.90 (d, J=13.7 Hz, 2H), 3.77-3.51 (m, 6H), 3.43 (dd, J=11.0, 7.1 Hz, 1H), 2.14 (ddd, J=12.6, 8.9, 6.9 Hz, 1H), 1.76 (ddd, J=12.7, 10.4, 8.7 Hz, 1H); MS (DCI) m/z 311.1 (M+H)⁺.

### Example 2-9

### (3S,5S)-5-({[tert-butyl(dimethyl)silyl]oxy}methyl)-3-(dibenzylamino)pyrrolidin-2-one

To a mixture of Example 2-8 (9.3 g, 30 mmol) and 1H-imidazole (2.2 g, 32 mmol) in N,N-dimethylformamide was added tert-butylchlorodimethylsilane (11.2 mL, 32.2 mmol, 50 weight % in toluene), and the reaction mixture was stirred overnight. The reaction mixture was partitioned between water and ethyl ether. The layers were separated, and the organic layer was washed with saturated aqueous sodium chloride. The combined aqueous layers were back extracted with diethyl ether. The combined organic layers were dried over anhydrous sodium sulfate, filtered, and concentrated under reduced pressure. The residue was purified by flash chromatography (silica gel, eluted with 35% ethyl acetate in heptane) to give the title compound (12.77 g, 99% yield). ¹H NMR (501 MHz, CDCl₃) δ ppm 7.50-7.43 (m, 4H), 7.37-7.30 (m, 4H), 7.29-7.22 (m, 2H), 6.00 (s, 1H), 4.01 (d, J=13.8 Hz, 2H), 3.75-3.66 (m, 4H), 3.56 (dddd, J=10.7, 7.0, 5.2, 2.2 Hz, 1H), 3.41 (dd, J=10.1, 7.9 Hz, 1H), 2.19 (dddd, J=12.9, 9.2, 6.8, 0.6 Hz, 1H), 1.71 (ddd, J=12.9, 10.5, 8.8 Hz, 1H), 1.30 (dt, J=4.0, 2.0 Hz, 1H), 0.90 (s, 9H), 0.98-0.80 (m, 2H), 0.07 (d, J=2.8 Hz, 6H); MS (DCI) m/z 425.1 (M+H)⁺.

### Example 2-10

### tert-butyl [(3S,5S)-5-({[tert-butyl(dimethyl)silyl]oxy}methyl)-3-(dibenzylamino)-2-oxopyrrolidin-1-yl]acetate

To a mixture of Example 2-9 (4.50 g, 10.6 mmol) in tetrahydrofuran (45 mL) stirring at 0° C. under nitrogen was added 95% sodium hydride (0.32 g, 12.67 mmol) in two portions. The cooled mixture was stirred for 40 minutes, and then tert-butyl 2-bromoacetate (4.22 g, 21.7 mmol, 3.2 mL) was added. The reaction mixture was warmed to ambient temperature and stirred overnight. The reaction mixture was quenched by the addition of water and ethyl acetate. The layers were separated, and the organic layer was washed with saturated aqueous sodium chloride. The combined aqueous layers were back extracted with ethyl acetate. The combined organic layers were dried over anhydrous sodium sulfate, filtered and concentrated under reduced pressure. The residue was purified by flash chromatography (silica gel, eluted with a gradient of 5-12% ethyl acetate in heptane) to give the title compound (5.3 g, 94% yield). ¹H NMR (400 MHz, CDCl₃) δ ppm 7.46-7.35 (m, 4H), 4.36 (d, J=17.5 Hz, 1H), 3.93 (d, J=13.8 Hz, 2H), 3.85-3.50 (m, 7H), 2.11 (ddd, J=12.8, 9.3, 6.8 Hz, 1H), 1.42 (s, 9H), 0.83 (s, 9H); MS (DCI) m/z 539.2 (M+H)⁺.

### Example 2-11

### tert-butyl [(3S,5S)-3-(dibenzylamino)-5-(hydroxymethyl)-2-oxopyrrolidin-1-yl]acetate

To a mixture of Example 2-10 (5.3 g, 9.8 mmol) in tetrahydrofuran (25 mL) was added tetrabutylammonium fluoride (11 mL, 11 mmol, 1.0 M in 95/5 tetrahydrofuran/water). The reaction mixture was stirred at ambient temperature for one hour and then quenched by the addition of saturated aqueous ammonium chloride mixture, water and ethyl acetate. The layers were separated, and the organic layer was washed with saturated aqueous sodium chloride. The combined aqueous layers were back extracted with ethyl acetate. The combined organic layers were dried over anhydrous sodium sulfate, filtered and concentrated under reduced pressure. The residue was purified by flash chromatography (silica gel, eluted with 35% ethyl acetate in heptane) to give the title compound (4.17 g, 100% yield). ¹H NMR (501 MHz, CDCl₃) δ ppm 7.50-7.41 (m, 4H), 7.36-7.28 (m, 4H), 7.28-7.21 (m, 2H), 4.40 (d, J=17.6 Hz, 1H), 3.96 (d, J=13.7 Hz, 2H), 3.78 (d, J=13.8 Hz, 3H), 3.71 (t, J=9.8 Hz, 1H), 3.61 (d, J=10.2 Hz, 1H), 3.55-3.45 (m, 2H), 3.40 (ddt, J=8.4, 7.5, 2.1 Hz, 1H), 2.36 (ddd, J=13.1, 9.9, 8.4 Hz, 1H), 2.12 (ddd, J=13.1, 9.6, 7.5 Hz, 1H), 1.51 (s, 9H); MS (DCI) m/z 425.1 (M+H)⁺.

### Example 2-12

### tert-butyl [(3S,5S)-3-(dibenzylamino)-2-oxo-5-(8,8,13,13-tetramethyl-5,5-dioxo-12,12-diphenyl-2,6,11-trioxa-5λ⁶-thia-12-silatetradecan-1-yl)pyrrolidin-1-yl]acetate

To a mixture of Example 2-11 (4.7 g, 11 mmol) in dimethyl sulfoxide (14 mL) was added a mixture of 4-((tert-butyldiphenylsilyl)oxy)-2,2-dimethylbutyl ethenesulfonate (14.5 g, 32.5 mmol, prepared as described in Organic and Biomolecular Chemistry 2007, 5(1), 132-138, S. Seeburger et al.) in dimethyl sulfoxide (14 mL). Potassium carbonate (2.60 g, 18.8 mmol) and water (0.028 g, 1.6 mmol, 0.028 mL) were added, and the reaction mixture was heated at 60° C. under nitrogen for one day. The reaction mixture was cooled to ambient temperature, and then quenched by the addition of saturated aqueous sodium chloride mixture, water and diethyl ether. The layers were separated, and the organic layer was washed with saturated aqueous sodium chloride. The combined aqueous layers were back extracted with diethyl ether. The combined organic layers were dried over anhydrous sodium sulfate, filtered and concentrated under reduced pressure. The residue was purified by flash chromatography (silica gel, eluted with a gradient of 15-25% ethyl acetate in heptane) to give the title compound (5.3 g, 55% yield). ¹H NMR (400 MHz, CDCl₃) δ ppm 7.71-7.63 (m, 2H), 7.43 (tdt, J=6.0, 4.3, 1.7 Hz, 4H), 7.39 (d, J=6.3 Hz, 1H), 7.35-7.27 (m, 2H), 7.27-7.19 (m, 1H), 4.20 (d, J=17.3 Hz, 0H), 4.00-3.88 (m, 2H), 3.88-3.77 (m, 1H), 3.80-3.66 (m, 2H), 3.67 (d, J=6.0 Hz, 1H), 3.55-3.48 (m, 1H), 3.27 (t, J=6.6 Hz, 1H), 2.18 (ddd, J=12.9, 9.3, 7.2 Hz, 0H), 1.69-1.55 (m, 2H), 1.44 (s, 4H), 1.05 (s, 4H), 0.96 (s, 3H); MS (ESI⁺) m/z 871.2 (M+H)⁺.

### Example 2-13

### tert-butyl [(3S,5S)-3-amino-2-oxo-5-(8,8,13,13-tetramethyl-5,5-dioxo-12,12-diphenyl-2,6,11-trioxa-5λ⁶-thia-12-silatetradecan-1-yl)pyrrolidin-1-yl]acetate

Example 2-12 (0.873 g, 0.50 mmol) was dissolved in ethyl acetate (5 mL) and methanol (15 mL), and palladium hydroxide on carbon (20% by weight,0.18 g) was added. The reaction mixture was stirred under a hydrogen atmosphere (30 psi) at ambient temperature for 30 hours and then at 50° C. for one hour. The reaction mixture was cooled to ambient temperature, filtered, and concentrated to give the title compound (0.673 g, 97% yield). ¹H NMR (400 MHz, CDCl₃) δ ppm 7.72-7.64 (m, 4H), 7.50-7.35 (m, 6H), 4.32 (d, J=17.5 Hz, 1H), 3.99 (s, 2H), 3.96-3.79 (m, 4H), 3.76 (t, J=6.6 Hz, 2H), 3.62-3.46 (m, 3H), 3.32 (t, J=6.5 Hz, 2H), 2.50 (ddd, J=12.7, 8.7, 6.8 Hz, 1H), 2.09 (s, 3H), 1.62 (t, J=6.7 Hz, 2H), 1.49-1.41 (m, 2H), 1.07 (s, 10H), 0.99 (d, J=2.7 Hz, 7H), 0.90 (s, 1H); MS (ESI⁺) m/z 691.0 (M+H)⁺.

### Example 2-14

### 4-{[(3S,5S)-1-(2-tert-butoxy-2-oxoethyl)-2-oxo-5-(8,8,13,13-tetramethyl-5,5-dioxo-12,12-diphenyl-2,6,11-trioxa-5λ⁶-thia-12-silatetradecan-1-yl)pyrrolidin-3-yl]amino}-4-oxobut-2-enoic acid

Maleic anhydride (0.100 g, 1.02 mmol) was dissolved in dichloromethane (0.90 mL), and a mixture of Example 2-13 (0.65 g, 0.941 mmol) in dichloromethane (0.90 mL) was added dropwise. The mixture was then heated at 40° C. for 2 hours. The reaction mixture was directly purified by flash chromatography (silica gel eluted with a gradient of 1.0-2.5% methanol in dichloromethane containing 0.2% acetic acid). After concentrating the title compound-bearing fractions, toluene (10 mL) was added, and the mixture was concentrated again to give the title compound (0.563 g, 76% yield). ¹H NMR (400 MHz, CDCl₃) δ ppm 7.82 (d, J=7.1 Hz, 1H), 7.72-7.65 (m, 4H), 7.50-7.37 (m, 6H), 7.32-7.24 (m, 1H), 7.20 (d, J=7.6 Hz, 1H), 6.43-6.31 (m, 2H), 4.73 (dt, J=9.8, 7.2 Hz, 1H), 4.40 (d, J=17.6 Hz, 1H), 4.02 (s, 2H), 4.00 (s, 1H), 3.89 (tt, J=11.1, 5.8 Hz, 2H), 3.78 (d, J=6.5 Hz, 2H), 3.74 (d, J=8.9 Hz, 1H), 3.63 (dd, J=10.2, 2.3 Hz, 1H), 3.52 (dd, J=10.3, 4.3 Hz, 1H), 3.39-3.22 (m, 2H), 2.81 (ddd, J=13.6, 9.8, 8.0 Hz, 1H), 2.38 (s, 1H), 1.79 (dt, J=13.6, 6.8 Hz, 1H), 1.62 (t, J=6.6 Hz, 2H), 1.49 (s, 9H), 1.07 (s, 9H), 1.00 (s, 5H), 0.99 (s, 1H); MS (ESI⁻) m/z 787.3 (M⁻H)⁻.

### Example 2-15

### tert-butyl [(3S,5S)-3-(2,5-dioxo-2,5-dihydro-1H-pyrrol-1-yl)-2-oxo-5-(8,8,13,13-tetramethyl-5,5-dioxo-12,12-diphenyl-2,6,11-trioxa-5λ⁶-thia-12-silatetradecan-1-yl)pyrrolidin-1-yl]acetate

Example 2-14 (0.56 g, 0.71 mmol) was slurried in toluene (7 mL), and triethylamine (0.16 g, 1.6 mmol, 0.22 mL) and sodium sulfate (0.525 g, 3.70 mmol) were added. The reaction mixture was heated under reflux under a nitrogen atmosphere for 6 hours, and the reaction mixture was then stirred at ambient temperature overnight. The reaction mixture was filtered, and rinsed with ethyl acetate. The eluent was concentrated under reduced pressure, and the residue was purified by flash chromatography (silica gel, eluted with 45/55 heptane/ethyl acetate) to give the title compound (0.363 g, 66% yield). ¹H NMR (501 MHz, CDCl₃) δ ppm 7.72-7.66 (m, 4H), 7.50-7.43 (m, 2H), 7.45-7.36 (m, 4H), 6.74 (s, 2H), 4.84 (dd, J=10.6, 9.7 Hz, 1H), 4.43 (d, J=17.4 Hz, 1H), 4.09-3.99 (m, 1H), 4.00 (s, 2H), 3.91 (d, J=9.2 Hz, 1H), 3.91-3.84 (m, 2H), 3.76 (t, J=6.7 Hz, 2H), 3.72 (dd, J=10.1, 7.5 Hz, 1H), 3.59 (dd, J=10.0, 3.1 Hz, 1H), 3.35 (t, J=6.3 Hz, 2H), 2.43 (ddd, J=12.5, 9.7, 7.1 Hz, 1H), 1.97 (ddd, J=12.5, 10.6, 9.1 Hz, 1H), 1.66-1.59 (m, 4H), 1.49 (s, 9H), 1.29 (s, 2H), 1.11-1.05 (m, 1H), 1.07 (s, 9H), 1.00 (s, 6H), 0.94-0.88 (m, 1H), 0.91-0.83 (m, 1H), 0.86 (s, 1H); MS (ESI⁺) m/z 771.6 (M+H)⁺.

### Example 2-16

### {(3S,5S)-3-(2,5-dioxo-2,5-dihydro-1H-pyrrol-1-yl)-2-oxo-5-[(2-sulfoethoxy)methyl]pyrrolidin-1-yl}acetic acid

Example 2-15 (1.2 g, 1.6 mmol) was dissolved in trifluoroacetic acid (22.2 g, 195 mmol, 15 mL) and heated to 65-70° C. under nitrogen overnight. The trifluoroacetic acid was removed under reduced pressure. The residue was dissolved in acetonitrile (2.5 mL) and purified by preparative reverse-phase liquid chromatography on a Phenomenex® Luna® C18(2) AXIA™ column (250×50 mm, 10 μm particle size) using a gradient of 5-75% acetonitrile containing 0.1% trifluoroacetic acid in water over 30 minutes, to give the title compound.(0.30 g, 51% yield). ¹H NMR (400 MHz, methanol-d₄) δ ppm 6.85 (d, J=1.0 Hz, 2H), 4.86 (t, J=10.1 Hz, 1H), 4.30 (dd, J=17.6, 8.0 Hz, 1H), 4.20 (dd, J=21.6, 17.6 Hz, 1H), 4.00 (dh, J=12.3, 3.2 Hz, 1H), 3.89-3.72 (m, 2H), 3.72-3.56 (m, 2H), 3.15-3.07 (m, 1H), 3.11-3.02 (m, 1H), 2.66 (s, 1H), 2.52-2.39 (m, 1H), 2.00-1.86 (m, 1H); MS (ESI⁻) m/z 375.2 (M−H)⁻.

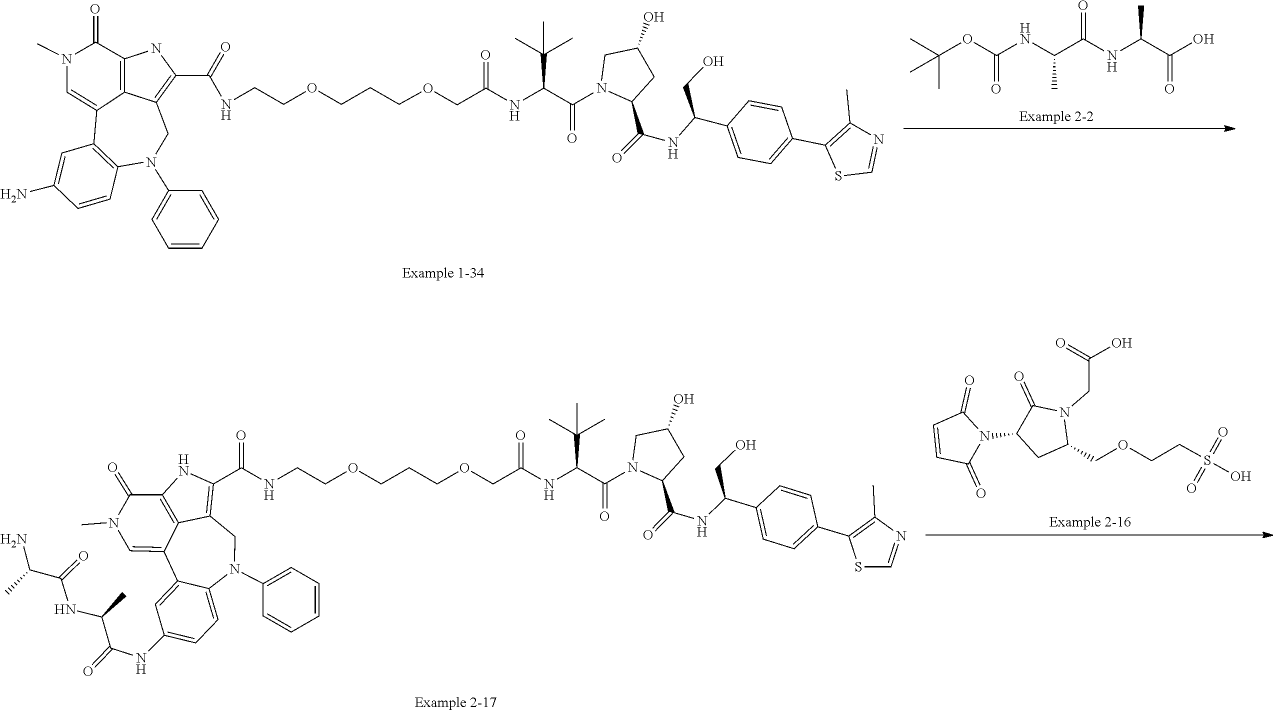

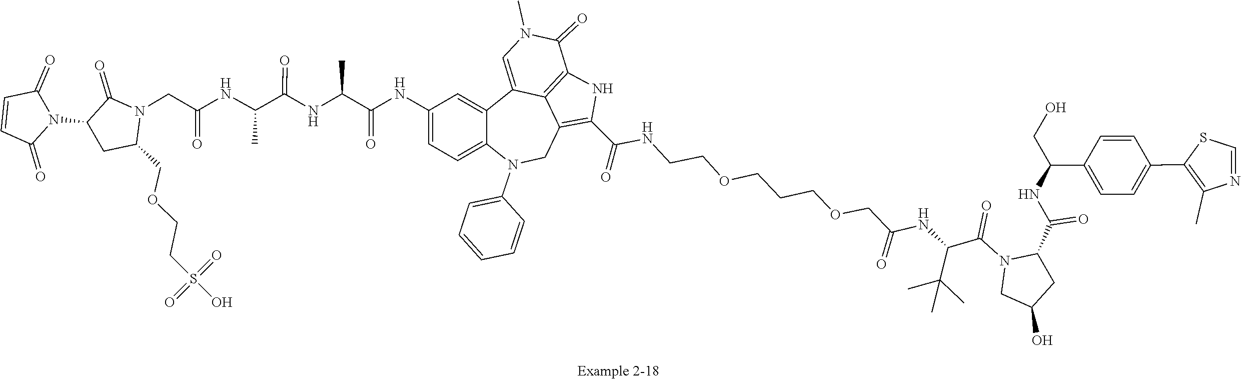 [SMILES: Cc1ncsc1-c1ccc([C@H](CO)NC(=O)[C@@H]2C[C@@H](O)CN2C(=O)[C@@H](NC(=O)COCCCOCCNC(=O)c2[nH]c3c(=O)n(C)cc4c3c2CN(c2ccccc2)c2ccc(NC(=O)[C@H](C)NC(=O)[C@H](C)NC(=O)CN3C(=O)[C@@H](N5C(=O)C=CC5=O)C[C@H]3COCCS(=O)(=O)O)cc2-4)C(C)(C)C)cc1]

### Example 2-17

### N-({3-[2-({10-[(L-alanyl-L-alanyl)amino]-2-methyl-3-oxo-7-phenyl-3,4,6,7-tetrahydro-2H-2,4,7-triazadibenzo[cd, f]azulene-5-carbonyl}amino)ethoxy]propoxy}acetyl)-3-methyl-L-valyl-(4R)-4-hydroxy-N-{(1R)-2-hydroxy-1-[4-(4-methyl-1,3-thiazol-5-yl)phenyl]ethyl}-L-prolinamide

A mixture of Example 1-34 (164 mg, 0.166 mmol), Example 2-2 (60.9 mg, 0.234 mmol), 1-((dimethylamino)(dimethyliminio)methyl)-1H-[1,2,3]triazolo[4,5-b]pyridine 3-oxide hexafluorophosphate(V) (86 mg, 0.226 mmol, HATU), and 2,6-lutidine (60 μL, 0.515 mmol) in N,N-dimethylformamide (2 mL) and dichloromethane (4 mL) was stirred at ambient temperature for 1 h. The mixture was concentrated by rotary evaporation, then treated with 2,2,2-trifluoroacetic acid (2 mL, 26.0 mmol) in 4 mL of dichloromethane for 40 minutes at ambient temperature. The reaction mixture was concentrated, dissolved in 1:1 dimethyl sulfoxide:methanol (2 mL), and purified by preparative reverse-phase HPLC (Method A), providing 161.3 mg (72%) of the title compound (arbitrarily assigned as the bis trifluoroacetic acid salt). ¹H NMR (500 MHz, DMSO-d₆) δ ppm 12.27 (s, 1H), 10.35 (s, 1H), 8.99 (s, 1H), 8.74 (d, J=7.0 Hz, 1H), 8.51-8.38 (m, 1H), 8.08 (d, J=5.3 Hz, 2H), 7.99 (d, J=33.3 Hz, 1H), 7.78-7.59 (m, 1H), 7.54 (s, 1H), 7.47-7.27 (m, 4H), 6.94 (dd, J=8.7, 7.2 Hz, 2H), 6.48 (t, J=7.2 Hz, 1H), 6.43-6.33 (m, 2H), 5.92 (d, J=17.0 Hz, 1H), 4.86 (dt, J=8.4, 6.3 Hz, 1H), 4.62-4.43 (m, 2H), 4.40-4.21 (m, 2H), 3.98-3.84 (m, 12H), 3.64-3.54 (m, 5H), 2.46 (s, 3H), 2.08 (s, 3H), 1.85 (q, J=6.5 Hz, 2H), 1.79 (td, J=8.7, 4.4 Hz, 1H), 1.49-1.32 (m, 6H), 0.94 (s, 9H); MS (ESI⁻) m/z 1128.5 (M−H)⁻.

### Example 2-18

### N-[(3-{2-[(10-{[N-({(3S,5S)-3-(2,5-dioxo-2,5-dihydro-1H-pyrrol-1-yl)-2-oxo-5-[(2-sulfoethoxy)methyl]pyrrolidin-1-yl}acetyl)-L-alanyl-L-alanyl]amino}-2-methyl-3-oxo-7-phenyl-3,4,6,7-tetrahydro-2H-2,4,7-triazadibenzo[cd, f]azulene-5-carbonyl)amino]ethoxy}propoxy)acetyl]-3-methyl-L-valyl-(4R)-4-hydroxy-N-{(1R)-2-hydroxy-1-[4-(4-methyl-1,3-thiazol-5-yl)phenyl]ethyl}-L-prolinamide

Example 2-16 (10 mg, 0.027 mmol), N-ethyl-N-isopropylpropan-2-amine (20 μL, 0.116 mmol), and 2-(3H-[1,2,3]triazolo[4,5-b]pyridin-3-yl)-1,1,3,3-tetramethylisouronium hexafluorophosphate(V) (11 mg, 0.029 mmol) were combined in N,N-dimethylformamide (0.5 mL). The reaction mixture was stirred at ambient temperature for 5 minutes, then added to a solution of Example 2-17 (37.2 mg, 0.027 mmol) and N-ethyl-N-isopropylpropan-2-amine (20 μL, 0.116 mmol) in N,N-dimethylformamide (0.5 mL). The mixture was stirred at ambient temperature for 1 hour, diluted with 2 mL of 1:1 dimethyl sulfoxide/methanol, filtered through a syringe filter (0.45 μm polypropylene), and then purified by preparative HPLC (Method A, eluted with 20-80% acetonitrile in water/0.1% trifluoroacetic acid) to give the title compound (15 mg, 35%). ¹H NMR (400 MHz, DMSO-d₆) δ ppm 12.18 (s, 1H), 9.93 (s, 1H), 9.85 (s, 1H), 8.95 (s, 1H), 8.44-8.35 (m, 2H), 8.22 (d, J=9.3 Hz, 1H), 8.03 (d, J=13.5 Hz, 2H), 7.70 (d, J=10.0 Hz, 1H), 7.55 (d, J=27.2 Hz, 1H), 7.46-7.28 (m, 5H), 7.23 (d, J=8.5 Hz, 1H), 7.03 (d, J=3.8 Hz, 2H), 6.88 (t, J=7.8 Hz, 2H), 6.42 (t, J=7.3 Hz, 1H), 6.37 (m, 1H), 5.87 (d, J=17.0 Hz, 1H), 4.82 (q, J=6.7 Hz, 1H), 4.69 (m, 1H), 4.56-4.40 (m, 2H), 4.42-4.29 (m, 2H), 4.27-4.08 (m, 2H), 3.97-3.82 (m, 3H), 3.70-3.40 (bd, 14H), 2.78 (t, J=6.7 Hz, 2H), 2.42 (s, 3H), 2.03 (dd, J=12.9, 8.2 Hz, 1H), 1.90-1.69 (m, 3H), 1.31 (d, J=7.2 Hz, 3H), 1.27-1.19 (m, 5H), 0.90 (s, 9H); MS (ESI⁻) m/z 1486.8 (M−H)⁻.

### Example 3. In Vitro Binding Assay

A TR-FRET binding assay was used to assess the binding properties of BET degraders. A comparative BET family bromodomain inhibitor, MS417 ((Zhang G et al. Journal of Biological Chemistry 2012, Vol 287, 28840-28851), CAS Registry number 916489-36-6) was used. MS417 was Alexa647-labeled and diluted in FRET assay buffer (20 mM sodium phosphate, pH 6.0, 50 mM NaCl, 1 mM ethylenediaminetetraacetic acid disodium salt dihydrate, 0.01% Triton X-100, 1 mM DL-dithiothreitol) for use. The target proteins for the assay were BRD4 (UniProt accession number: 060885-1) polypeptides. The components of the binding assay were the His-tagged BRD4 Bromo domain1 (K57-E168) (SEQ ID NO: 18) or the His-tagged BRD4 Bromodomain 2 (E352-M457) (SEQ ID NO: 19), a europium-conjugated anti-His antibody (Invitrogen/ThermoFisher), Example 1 (BETd1), and the Alexa647-conjugated MS417. After a one-hour equilibration at ambient temperature, TR-FRET ratios were determined using an Envision multi-label plate reader (PerkinElmer, Waltham, MA, USA) at a europium excitation wavelength of 340 nm and Alexa647 probe emission was monitored at 665 nm. BETd1 displayed an IC₅₀ of 0.0102 μM against BRD4 bromo domain1 and 0.0506 μM against BRD4 bromo domain2.

### Example 4. CD33 mAb1

A rat antibody that binds to human CD33 protein, human monocytes, cynomolgus monkey monocytes, and recombinant HEK-293T cells expressing CD33 was isolated using hybridoma technology. The rat antibody was used to generate mAb1.

mAb1 is a recombinant, humanized, IgG1 kappa monoclonal antibody that binds to CD33. mAb1 consists of humanized complementarity determining regions of the chimeric rat antibody fused with human immunoglobulin gamma 1 heavy chain (Y296C, N297A) and kappa light chain constant regions.

Protein engineering was carried out for site specific conjugation. The following are found in the CH2 domain of the heavy chain, in the Fc region: Y296C is the site of conjugation; N297A removes the native glycosylation site. Conjugation at a cysteine (Y296C), in the absence of glycan (N297A), can direct the attached linker-drug into the pocket normally occupied by the glycan. Sequestration of the linker-drug in this pocket decreases the apparent hydrophobicity of the ADC, indicating the linker-drug is shielded from the aqueous environment. The resulting variant was selected as mAb1 [mutations: G54A CDR-H2; S62A CDR-H2; G28D CDR-L1; S93E CDR-L3]. The antibodies are human IgG1/k isotype, with z, non-a allotype.

Representative lots of unconjugated anti-human CD33 antibody mAb1 were produced via stable CHO pool expression and purified.

Peptide mapping was carried out to determine modifications or deletions to the amino acids present in the heavy and light chains of mAb1. Following denaturation and reduction, the mAb was subjected to digestion using the proteolytic enzyme, Lys-C, which cleaves peptide bonds on the C-terminal end of lysine. Digested samples were analyzed using reduced reverse phase liquid chromatography-mass spectrometry (RPLC-MS) with Orbitrap MS under denaturing conditions to monitor the major fragments for post translational modifications.

Analysis of three batches of mAb1 peptide fragments following proteolytic digestion showed that two species of the heavy chain were present. Quantitation was performed based on the signal intensity of the extracted ion chromatogram of the digested peptide with and without the C-terminal lysine. Only a small percentage (1.7 percent) of the heavy chain of mAb1 consisted of heavy chains with a C-terminal lysine, as is shown in SEQ ID No: 9. The predominant species of the heavy chain in mAb1 lacked the C-terminal lysine, as is shown in SEQ ID No: 10. The light chain-specific fragments following mAb1 digestion were consistent across lots as expected from the encoded primary amino acid sequence, as is shown in SEQ ID No: 11.

### Example 5. mAb1 Specific Binding to CD33-Expressing HEK-293 Cells

To determine the binding of the anti-human CD33 mAb1, flow cytometry analysis was used. Control and CD33-expressing HEK-293 cells were harvested from flasks when approximately 80% confluent using Cell Dissociation Buffer (Life Technologies/ThermoFisher, Carlsbad, CA, USA). Cells were washed once in PBS/1% FBS (FACS buffer), resuspended at 1.5-2.0×10⁶ cells/mL in FACS buffer and transferred to a round-bottom plate (Corning) at 100 μL/well. Ten microliters of a 10× concentration of tested antibodies were added and plates were incubated at 4° C. for 2 hours. Wells were washed twice with FACS buffer and resuspended in 50 mL of 1:500 anti-human IgG Ab (AlexaFluor 488, Invitrogen/ThermoFisher, Carlsbad, CA, USA) diluted in FACS buffer. Plates were incubated at 4° C. for 1 hour and washed twice with FACS buffer. Cells were resuspended in 100 mL of PBS/1% formaldehyde and analyzed on a Becton Dickinson (Franklin Lakes, NJ, USA) LSRII flow cytometer.

A negative control human anti-CMV IgG mAb2 and mAb1 were tested for CD33 specific binding in a FACS titration experiment on control and CD33 expressing HEK-293 cells. The anti-human CD33 mAb1 binds specifically to the CD33-expressing HEK-293 cells but not the control HEK-293 cells. No binding was observed with the control mAb2 that recognizes CMV (cytomegalovirus), which is not present in the model used. The antibody mAb2 having a heavy chain sequence as set forth in SEQ ID NO: 14 and a light chain sequence as set forth in SEQ ID NO: 15 was tested.

### Example 6. Conjugation Procedure to Produce mAb1-LD2 ADC

mAb1 was conjugated to a linker drug (Example 2-1, LD1) to form antibody-drug conjugate mAb1-LD2, a representative ADC of Formula (I).

The antibody from Example 4, mAb1, was reduced with an excess of reducing agent, 4-(diphenylphosphino)benzoic acid (DpHPBA), as described below and then conjugated with an excess of drug-linker.

To remove oxygen from the reaction solution, the reaction headspace was swept with nitrogen and mixed until the solution concentrations measured <1 ppm. To reduce the antibody, 2.5 molar equivalents of 4-(diphenylphosphino)benzoic acid in dimethyl sulfoxide (0.3 w/w %) was added and mixed at 4° C. overnight (16-24 hours). The reduced antibody was reacted with linker-drug LD1 from Example 2-1 (2.05 molar equivalents as a 0.8 w/w % solution in dimethyl sulfoxide) and reacted at 20° C. for approximately 1 hour. The reaction mixture was purified by hydrophobic interaction chromatography. Product fractions were concentrated and buffer and pH adjusted to pH 8.5 to effect opening of the linker ring (held for 48 hours at 20° C.). The ring-opened product solution was sterile filtered and stored at −80° C.

### Example 7. Conjugation Procedure to Produce mAb1-LD3 ADC

mAb1 was conjugated to a linker drug (Example 2-1, LD1) to form antibody-drug conjugate mAb1-LD3, a representative ADC of Formula (VI). To reduce the CD33 antibody with HC:Y296C, N297A mutation (mAb1), 3 molar equivalents of 4-(diphenylphosphino)benzoic acid was added into the aqueous antibody solution, and the reaction mixture was vortexed gently. The reaction mixture was incubated at 4° C. overnight (16-24 hours). For the conjugation reaction, 2.1-2.5 molar equivalents of linker-drug (Example 2-1, LD1) (dissolved as 10 mM stock solution in N,N-dimethylacetamide (DMA),) was added to the reduced antibody and gently mixed, followed by incubation at 4° C. for 1 hour. The conjugate was purified and filtered through a 0.2 μM filter (Acrodisc Syringe Filter, Pall Corporation, Port Washington, NY, USA) and stored at 4° C.

### Example 8. Characterization of DAR (Drug Antibody Ratio) and Percentage of Aggregation of ADCs Determinations

DAR determination by LC-MS analysis mAb1-LD2: Reduced subunit analysis was performed using a ThermoFisher Vanquish LC coupled to an Orbitrap Fusion Lumos mass spectrometer (Waltham, MA). The ADC was reduced using 32 mM (final concentration)dithiothreitol at room temperature for 30 minutes, separated using a BioResolve RP mAb Polyphenyl column (2.1×150 mm, 2.7 μm dp, 450 Å pore size, Waters Corporation, Milford, MA, USA). Mobile phase A was 0.085% formic acid and 0.015% trifluoroacetic acid in H₂O, mobile phase B was 0.085% formic acid and 0.015% trifluoroacetic acid in acetonitrile, and flow rate was 0.4 mL/minute. The extracted intensity vs. m/z spectrum was deconvoluted using Protein Metrics Byos Intact Module (Dotmatics, Boston, MA, USA) to determine the mass of each reduced antibody fragment. DAR was calculated from the deconvoluted spectrum by summing intensities of the unmodified and modified peaks for the heavy chain, normalized by multiplying intensity by the number of drugs attached. The summed, normalized intensities were divided by the sum of the intensities, and the summing results for two heavy chains produced a final average DAR value for the full ADC. Native intact analysis was performed using a Waters UHPLC H-Class Acquity coupled to a Xevo G2-XS Q-TOF mass spectrometer. The ADC was separated by Acquity UPLC Protein BEH SEC column (4.6×150 mm, 1.7 μm dp, 200 Å pore size, Waters), and the mobile phase of 100 mM ammonium acetate in 10% isopropanol was used. The flow rate was 0.2 mL/minute, and the mass spectrum was acquired by MassLynx software (Waters). The extracted mass spectrum was deconvoluted using Protein Metrics Byos Intact Module (Dotmatics) and to determine the DAR profile. DAR 2 is the dominant form of mAb1-LD2.

DAR determination by LC-MS analysis mAb1-LD3: Analysis was performed using an Agilent 1100 HPLC system interfaced to an Agilent LC/MSD TOF 6220 ESI mass spectrometer. The ADC was reduced with 25 mM (final concentration) Bond-Breaker® TCEP solution (Thermo Scientific, Rockford, IL, USA), loaded onto a Protein Microtrap (Michrom Bioresources, Auburn, CA, USA) desalting cartridge, and eluted with a gradient of 10% B to 75% B in 0.2 minutes at ambient temperature. Mobile phase A was H₂O with 0.1% formic acid, mobile phase B acetonitrile with 0.1% formic acid, and flow rate was 0.2 mL/minute. Electrospray-ionization time-of-flight mass spectra of the co-eluting light and heavy chains were acquired using Agilent MassHunter™ acquisition software (Agilent, Santa Clara, CA, USA). The extracted intensity vs. m/z spectrum was deconvoluted using the Maximum Entropy feature of MassHunter™ software to determine the mass of each reduced antibody fragment. DAR was calculated from the deconvoluted spectrum by summing intensities of the naked and modified peaks for the light chain and heavy chain, normalized by multiplying intensity by the number of drugs attached. The summed, normalized intensities were divided by the sum of the intensities, and the summing results for two light chains and two heavy chains produced a final average DAR value for the full ADC of about 2.

### Example 9. ADC Characterization

Two representative lots of ADC were made by conjugation at the engineered Y296C cysteine of antibody produced via stable CHO pool expression using a selective reduction protocol. These lots were characterized to determine drug-antibody ratio (DAR) and specificity of conjugation by mass spectroscopy, aggregate content by size exclusion chromatography (SEC), and apparent hydrophobicity by hydrophobic interaction chromatography (HIC). The starting antibody lots were mostly monomeric (˜94%), and the engineered cysteines were approximately 90% fully capped with either cysteine or glutathione. The resultant ADCs retained high monomer content (˜99%), with DAR2 as the predominant form. The conjugation was highly selective as witnessed by >95% of the heavy chains having the expected single modification of 1 molecule of LD2 per heavy chain.

Confirmation of the conjugation sites of mAb1-LD2 was accomplished by Lys-C digestion of reduced and alkylated antibody followed by reversed-phase HPLC separation of the resulting peptides. The conjugated peptides were identified using electrospray ionization mass spectrometry (ESI-MS) and compared with the theoretical monoisotopic molecular weights. The heavy chain fragment (HC 289-317) has a theoretical mass of 4862.29 Da and an observed mass of 4862.30 Da confirming the expected conjugation site at C296.

### Example 10. Cell Culture

AML cell lines described in the following examples were originally obtained from ATCC (American Type Culture Collection) or DSMZ (Deutsche Sammlung von Mikroorganismen und Zellkulturen). All AML cell lines were grown in RPMI-1640+10% FBS (Gibco/Thermo Fisher, Waltham, MA, USA) and were maintained in culture for less than two months after thawing. Primary AML cells were maintained in StemSpan™ SFEM media (STEMCELL™ Technologies, Vancouver, CA)+cytokine cocktail.

### Example 11. Anti-Proliferative Activity of mAb1-LD2, mAb1-LD3 ADCs in AML Cell Lines Cell Proliferation Assay Following Treatment with mAb3-LD3, mAb1-LD2, and mAb1-LD3

Cells were plated into 384-well plates in their respective culture media and incubated at 37° C. at an atmosphere of 5% CO₂. A serial 1:3 dilution ranging from 30 nM to 0.005 nM of ADCs were prepared. The ADCs used included a control ADC, which was an anti-tetanus toxin mAb conjugated to the BET degrader linker drug LD1 and designated mAb3-LD3. mAb3 has a heavy chain sequence as set forth in SEQ ID NO: 16 and a light chain sequence as set forth in SEQ ID NO: 17.

In addition to mAb3-LD3, the ADCs mAb1-LD2, and mAb1-LD3 as described previously were used in these assays. The diluted ADCs were added to the plates after 24 hours growth and cell cultures were incubated for three additional days at 37° C. The Celltiter-Glo® assay (Promega, Madison, WI, USA) was performed according to manufacturer's instructions to determine cell proliferation. A luminescence signal from each well was acquired using the Victor 3 plate reader (PerkinElmer, Waltham, MA, USA), and the data was analyzed using the GraphPad Prism software (Dotmatics).

TABLE 5
Proliferation of AML cell lines following
treatment with ADCs, EC₅₀ (nM)*

| AML Cell Line | mAb3-LD3 | mAb1-LD2 | mAb1-LD3 |
|---|---|---|---|
| AML2 | 12.29 | <0.005 | <0.005 |
| MOLM13 | 14.38 | 0.005 | 0.005 |
| MV411 | 9.29 | 0.005 | <0.005 |
| THP1 | 20.27 | 0.039 | 0.073 |

*EC₅₀: the concentration (or dose) effective in producing 50% of the maximal response

mAb3-LD3 was largely inactive at preventing proliferation of AML cell lines, whereas, both mAb1 ADCs significantly limited cell proliferation in the AML cell lines tested.

BRD4 Quantitation as Affected by mAb3-LD3, mAb1-LD2, and mAb1-LD3

Cells were plated into 96-well plates in their respective culture media and incubated at 37° C. in an atmosphere of 5% CO₂ overnight. A serial 1:3 dilution of ADCs was prepared and added to the plate the next day. After incubation with ADCs for 18-hours, the cells were frozen and stored at −80° C. for later assay. On the day of MSD protein assay (Mesoscale Discovery, Rockville, MD, USA), cells were lysed on ice with 100 μL of lysis buffer (RIPA buffer: Thermo Scientific, protease inhibitor cocktail tablet: Roche, and PMSF) added per well and the amounts of BRD4 protein in each well were determined using the anti-BRD4 rabbit mAb capture antibody (Fortis Life Sciences, Waltham, MA, USA) following the MSD manufacturer's suggested protocol using an MSD plate reader (Meso Scale Discovery), and the data was analyzed using the GraphPad Prism software (Dotmatics).

TABLE 6
BRD4 quantitation in AML cell lines following treatment by ADCs
and measured by MSD: expressed as DC₅₀ (nM)* concentration.

| AML Cell Line | mAb3-LD3 | mAb1-LD2 | mAb1-LD3 |
|---|---|---|---|
| AML2 | >10 | 0.041 | 0.047 |
| MOLM13 | >10 | 0.038 | 0.043 |
| MV411 | 5.802 | 0.020 | 0.021 |
| THP1 | 4.142 | 0.016 | 0.018 |

*DC₅₀ is the half maximal degradation concentration (See Semenova E, et al. Flexible Fitting of PROTAC Concentration-Response Curves with Changepoint Gaussian Processes. SLAS Discovery 2021 October; 26(9): 1212-1224).

The negative control anti-tetanus toxin mAb3-LD3 was largely inactive at affecting BRD4 protein in AML cell lines, whereas, both mAb1 ADCs significantly decreased the presence of BRD4 in the AML cell lines tested.

### Example 12. mAb1-LD3 Activity in Patient Derived Xenograft (“PDX”) Systemic Models

For the engraftment study, AML patient PBMC (peripheral blood mononuclear cell) samples were used to intra-hepatically inoculate sub-lethally irradiated neonate immunodeficient CIEA-NOG mice (Taconic Biosciences, Rensselaer, NY, USA). After 12 weeks, engraftment was confirmed in surrogate mice, and the remaining cohorts were screened and randomized into treatment groups. After five weeks of treatment, the presence of human cells in bone marrow was assessed by flow cytometry.

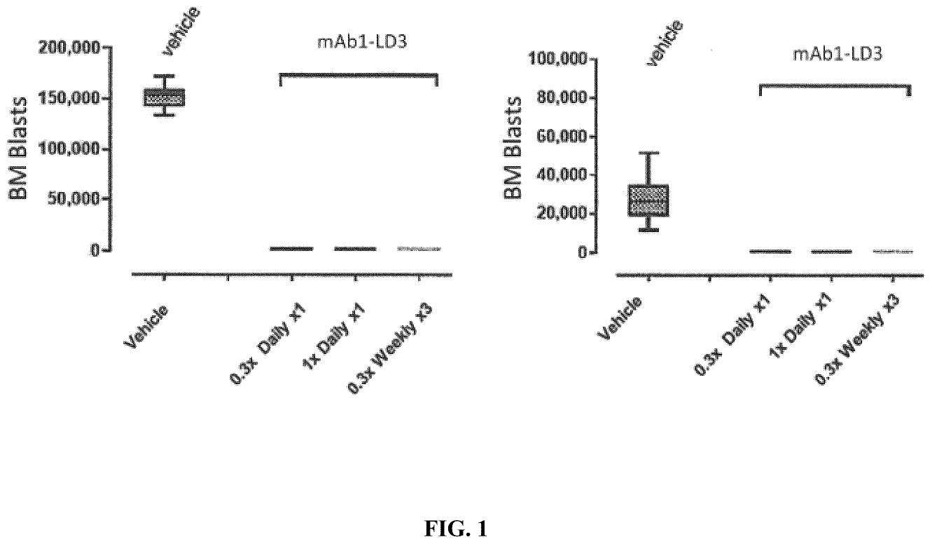

The anti-proliferative activity of mAb1-LD3 was evaluated on AML patient isolates by PDX transplants of PBMC into immunosuppressed mice. Mice were dosed with mAb1-LD3 ADC in three separate cohorts. The proliferation of human cells in the bone marrow of the mice in each cohort was found to be inhibited when compared to a negative control. The results of mAb1-LD3 are depicted in FIG. 1.

### Example 13. Efficacy Displayed by mAb1-LD2 and mAb1-LD3 in CDX Mouse Models

Engraftment model: female NSG mice are purchased from Jackson Laboratories (Bar Harbor, ME, USA) and used for tumor engraftment models with a bioluminescent readout, as described below. Mice are 6-8 weeks of age and weigh between 21-24 grams at the initiation of the study. Food and water are provided ad libitum, and vivarium rooms are kept on a 12 hour light phase: 12 hour dark phase schedule. All animal studies are conducted in accordance with the guidelines established by the AbbVie Institutional Animal Care and Use Committee.

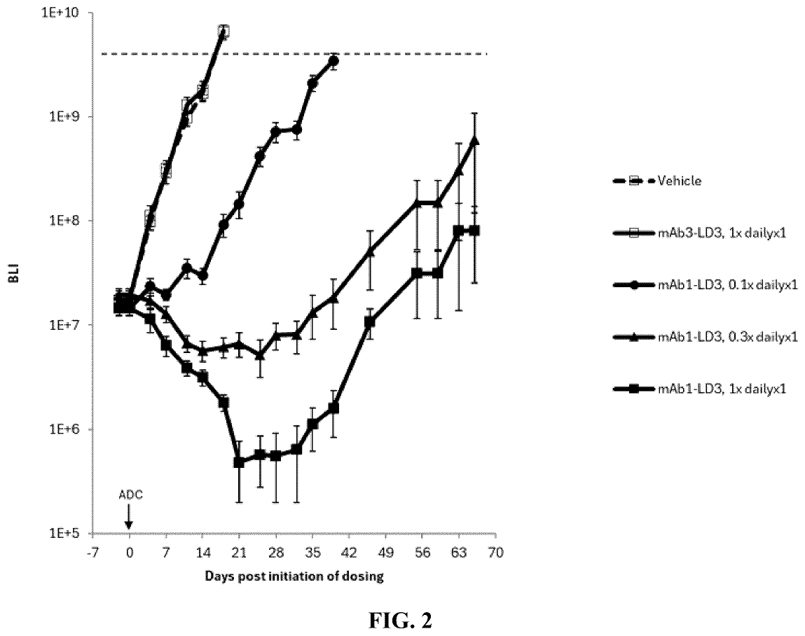

In the systemic engraftment model, the Red-Fluc stable F.luciferase-expressing cell lines are generated by infecting the parental AML cell lines (MOLM13 from DSMZ, Braunschweig, DE, and THP-1 and MV 4-11, both from the ATCC, Manassas, VA, USA) with the RediFect Red-FLuc-Puromycin lentiviral particles (PerkinElmer, Waltham, MA), creating stable cell lines which are validated for engraftment parameters and for stability of signal. Mice are then inoculated with MOLM13-Red-Fluc (1×10⁶ cells), THP1-Red-Fluc (1×10⁶ cells) and MV4-11-Red-Fluc (5×10⁶ cells) intravenously into the tail vein with 0.2 mL inoculum of culture media. To measure tumor engraftment, bioluminescence (BLI) signals are obtained 10 minutes after IP injection of luciferin (150 mg/kg) using a LagoX® imaging system (Spectral Instruments Imaging, Tucson, AZ, USA). Signal intensity is determined by examining regions of interest around the whole image of the mouse. Engraftment data are quantified using LagoX® software and mice are allocated at 3×10⁶ signal into treatment and control groups, with 9 mice per study group. Tumor volume measurements are estimated based on normalized BLI measurements, as BLI=(Treated mouse BLI−Background at size match BLI). Mice are administered either mAb1-LD2 or mAb1-LD3 as a single intraperitoneal dose, at three different predetermined doses. BLI signal is monitored at least twice weekly with the mice in dorsal and ventral positions. Mice are removed from the study and humanely euthanized when a BLI signal of 1×10¹⁰ BLI was observed. The results for mAb1-LD3 are depicted in FIG. 2.

SEQUENCE LISTING TABLE

| SEQ | | |
|---|---|---|
| ID NO: | Description | Sequence |
| 1 | mAb1 CDR-H1 | GFTLSDYAMA |
| 2 | mAb1 CDR-H2 | TISYDAGRTYYRDAVKG |
| 3 | mAb1 CDR-H3 | PPGVYYGSY |
| 4 | mAb1 CDR-L1 | RASQDISNFLN |
| 5 | mAb1 CDR-L2 | YTSNLQS |
| 6 | mAb1 CDR-L3 | QQYDESPPT |
| 7 | mAb1 Heavy Chain | EVQLVESGGGVVQPGRSLRLSCAASGFTLSDYAMAW |
| | Variable Region | VRQAPGKGLEWVATISYDAGRTYYRDAVKGRFTISRD |
| | | NSKNTLYLQMNSLRAEDTAVYYCERPPGVYYGSYWG |
| | | QGTMVTVSS |
| 8 | mAb1 Light Chain | DIQMTQSPSSLSASVGDRVTITCRASQDISNFLNWYQQ |
| | Variable Region | KPGKVPKLLIYYTSNLQSGVPSRFSGSGSGTDFTLTISS |
| | | LQPEDVATYYCQQYDESPPTFGQGTKLEIK |
| 9 | mAb1 Heavy Chain | EVQLVESGGGVVQPGRSLRLSCAASGFTLSDYAMAW |
| | | VRQAPGKGLEWVATISYDAGRTYYRDAVKGRFTISRD |
| | | NSKNTLYLQMNSLRAEDTAVYYCERPPGVYYGSYWG |
| | | QGTMVTVSSASTKGPSVFPLAPSSKSTSGGTAALGCLV |
| | | KDYFPEPVTVSWNSGALTSGVHTFPAVLQSSGLYSLSS |
| | | VVTVPSSSLGTQTYICNVNHKPSNTKVDKKVEPKSCD |
| | | KTHTCPPCPAPELLGGPSVFLFPPKPKDTLMISRTPEVT |
| | | CVVVDVSHEDPEVKFNWYVDGVEVHNAKTKPREEQC |
| | | ASTYRVVSVLTVLHQDWLNGKEYKCKVSNKALPAPIE |
| | | KTISKAKGQPREPQVYTLPPSREEMTKNQVSLTCLVKG |
| | | FYPSDIAVEWESNGQPENNYKTTPPVLDSDGSFFLYSK |
| | | LTVDKSRWQQGNVFSCSVMHEALHNHYTQKSLSLSP |
| | | GK |
| 10 | mAb1 Heavy Chain | EVQLVESGGGVVQPGRSLRLSCAASGFTLSDYAMAW |
| | lacking C- | VRQAPGKGLEWVATISYDAGRTYYRDAVKGRFTISRD |
| | terminal lysine | NSKNTLYLQMNSLRAEDTAVYYCERPPGVYYGSYWG |
| | | QGTMVTVSSASTKGPSVFPLAPSSKSTSGGTAALGCLV |
| | | KDYFPEPVTVSWNSGALTSGVHTFPAVLQSSGLYSLSS |
| | | VVTVPSSSLGTQTYICNVNHKPSNTKVDKKVEPKSCD |
| | | KTHTCPPCPAPELLGGPSVFLFPPKPKDTLMISRTPEVT |
| | | CVVVDVSHEDPEVKFNWYVDGVEVHNAKTKPREEQC |
| | | ASTYRVVSVLTVLHQDWLNGKEYKCKVSNKALPAPIE |
| | | KTISKAKGQPREPQVYTLPPSREEMTKNQVSLTCLVKG |
| | | FYPSDIAVEWESNGQPENNYKTTPPVLDSDGSFFLYSK |
| | | LTVDKSRWQQGNVFSCSVMHEALHNHYTQKSLSLSP |
| | | G |
| 11 | mAb1 Light Chain | DIQMTQSPSSLSASVGDRVTITCRASQDISNFLNWYQQ |
| | | KPGKVPKLLIYYTSNLQSGVPSRFSGSGSGTDFTLTISS |
| | | LQPEDVATYYCQQYDESPPTFGQGTKLEIKRTVAAPSV |
| | | FIFPPSDEQLKSGTASVVCLLNNFYPREAKVQWKVDN |
| | | ALQSGNSQESVTEQDSKDSTYSLSSTLTLSKADYEKHK |
| | | VYACEVTHQGLSSPVTKSFNRGEC |
| 12 | mAb1 Heavy Chain: | atggagttcggcctcagctggctgtttctggtcgctattctgaaaggcgtgcagtgtgaa |
| | DNA Sequence | gtccagctggtggagtccggaggaggagtggtccagcccggaaggtctctgaggct |
| | | gagctgtgccgctagcggatttacactgagcgactacgccatggcttgggtcagacaa |
| | | gcccccggcaaaggactggagtgggtggctacaatctcctacgacgctggaaggac |
| | | atactatagggacgccgtgaaaggaaggttcacaatctctagggacaactccaagaa |
| | | cacactgtacctccagatgaactctctgagagccgaggatactgccgtctactactgcg |
| | | aaaggcctcccggcgtctactatggcagctactggggacaaggcacaatggtgactg |
| | | tgtccagcgctagcacaaaaggcccatccgtctttcctctggcccctagcagcaaaag |
| | | cacttccggcggaactgctgctctcggatgtctggtcaaggattacttcccagagccag |
| | | tgacagtgagctggaacagcggagctctcacttccggcgtgcatactttcccagccgt |
| | | cctccaatccagcggactgtattctctgtcctccgtggtcactgtgccttccagcagcct |
| | | cggcacacagacttacatctgcaatgtgaatcacaagccttccaacacaaaggtcgac |
| | | aagaaggtcgagcctaaaagctgtgacaagactcacacatgccctccttgtccagctc |
| | | cagagctgctgggcggaccatccgtgttcctcttccctcctaagcctaaggacactctg |
| | | atgatctctaggactccagaggtcacatgtgtcgtggtcgacgtcagccacgaggacc |
| | | cagaggtgaaattcaactggtacgtcgacggcgtggaggtgcacaatgccaaaacta |
| | | agcctagggaggagcaatgtgccagcacatatagggtcgtgtccgtgctgacagtgc |
| | | tgcaccaagactggctcaacggaaaggagtacaagtgtaaggtctccaataaggccc |
| | | tcccagctccaatcgagaagactatctccaaggccaagggccagcctagggaacca |
| | | caagtgtacactctcccaccttctagggaagagatgacaaagaaccaagtgtctctga |
| | | cttgtctcgtcaagggcttctacccaagcgacattgctgtggagtgggaaagcaacgg |
| | | ccagccagaaaacaactacaagacaacaccaccagtgctcgacagcgatggcagct |
| | | tcttcctctactccaagctgacagtggacaagtctaggtggcagcaaggcaacgtgttc |
| | | agctgcagcgtcatgcacgaagctctccacaatcactacactcagaagtctctgtctct |
| | | gtcccccggcaagtga |
| 13 | mAb1 Light Chain: | atggacatgcgcgtgcccgcccagctgctgggcctgctgctgctgtggttccccggct |
| | DNA Sequence | cgcgatgcgacatccagatgacccagtctccatcctcactgtctgcatctgtaggagac |
| | | agagtcaccatcacttgtcgggcgagtcaggatattagcaatttcttaaattggtatcag |
| | | cagaaaccagggaaagtccctaagctcctgatctattatacatccaacttgcaaagtgg |
| | | ggtcccatcaaggttcagcggcagtggatctgggacagatttcactctcaccatcagc |
| | | agcctgcagcctgaagatgttgcaacttattactgccaacagtatgacgagagccctcc |
| | | cacttttggccaggggaccaagctggagatcaaacgtacggtggctgcaccatctgtc |
| | | ttcatcttcccgccatctgatgagcagttgaaatctggaactgcctctgttgtgtgcctgc |
| | | tgaataacttctatcccagagaggccaaagtacagtggaaggtggataacgccctcca |
| | | atcgggtaactcccaggagagtgtcacagagcaggacagcaaggacagcacctaca |
| | | gcctcagcagcaccctgacgctgagcaaagcagactacgagaaacacaaagtctac |
| | | gcctgcgaagtcacccatcagggcctgagctcgcccgtcacaaagagcttcaacag |
| | | gggagagtgttga |
| 14 | mAb2 Heavy Chain | EEQVLESGGGLVKPGGSLRLSCAASGFTFSPYSVFWVR |
| | | QAPGKGLEWVSSINSDSTYKYYADSVKGRFTISRDNA |
| | | ENSIFLQMNSLRAEDTAVYYCARDRSYYAFSSGSLSD |
| | | YYYGLDVWGQGTTVIVSSASTKGPSVFPLAPSSKSTSG |
| | | GTAALGCLVKDYFPEPVTVSWNSGALTSGVHTFPAVL |
| | | QSSGLYSLSSVVTVPSSSLGTQTYICNVNHKPSNTKVD |
| | | KKVEPKSCDKTHTCPPCPAPELLGGPSVFLFPPKPKDT |
| | | LMISRTPEVTCVVVDVSHEDPEVKFNWYVDGVEVHN |
| | | AKTKPREEQYNSTYRVVSVLTVLHQDWLNGKEYKCK |
| | | VSNKALPAPIEKTISKAKGQPREPQVYTLPPSREEMTK |
| | | NQVSLTCLVKGFYPSDIAVEWESNGQPENNYKTTPPV |
| | | LDSDGSFFLYSKLTVDKSRWQQGNVFSCSVMHEALH |
| | | NHYTQKSLSLSPGK |
| 15 | mAb2 Light Chain | DIVMTQSPLSLSVTPGEPASISCRSSQSLLHTNGYNYLD |
| | | WYVQKPGQSPQLLIYLASNRASGVPDRFSGSGSGTDFT |
| | | LKISRVETEDVGVYYCMQALQIPRTFGQGTKVEIKRTV |
| | | AAPSVFIFPPSDEQLKSGTASVVCLLNNFYPREAKVQW |
| | | KVDNALQSGNSQESVTEQDSKDSTYSLSSTLTLSKAD |
| | | YEKHKVYACEVTHQGLSSPVTKSFNRGEC |
| 16 | mAb3 Heavy Chain | EVQLLESGGDLVRPGGSLRLSCAASGFSFSRYGMSWV |
| | | RQAPGKGLDWVAHISASAGATYYADSVKGRFTISRDN |
| | | SKNTLFLQMNNLRADDTAIYYCAKGGKQWLIPWFDP |
| | | WGQGTLVTVSSASTKGPSVFPLAPSSKSTSGGTAALGC |
| | | LVKDYFPEPVTVSWNSGALTSGVHTFPAVLQSSGLYS |
| | | LSSVVTVPSSSLGTQTYICNVNHKPSNTKVDKKVEPKS |
| | | CDKTHTCPPCPAPELLGGPSVFLFPPKPKDTLMISRTPE |
| | | VTCVVVDVSHEDPEVKFNWYVDGVEVHNAKTKPREE |
| | | QCASTYRVVSVLTVLHQDWLNGKEYKCKVSNKALPA |
| | | PIEKTISKAKGQPREPQVYTLPPSREEMTKNQVSLTCL |
| | | VKGFYPSDIAVEWESNGQPENNYKTTPPVLDSDGSFFL |
| | | YSKLTVDKSRWQQGNVFSCSVMHEALHNHYTQKSLS |
| | | LSPGK |
| 17 | mAb3 Light Chain | DIQMTQSPSSVSASVGDRVTIACRASQDISDRLAWYQQ |
| | | KPGKVPKVLIYGASSLQSGVPSRFSGSGSGTDFTLTINS |
| | | LQPEDFATYYCQQANSFPLTFGGGTKVEMKRTVAAPS |
| | | VFIFPPSDEQLKSGTASVVCLLNNFYPREAKVQWKVD |
| | | NALQSGNSQESVTEQDSKDSTYSLSSTLTLSKADYEKH |
| | | KVYACEVTHQGLSSPVTKSFNRGEC |
| 18 | BRD4 Bromodomain 1 | KRQTNQLQYLLRVVLKTLWKHQFAWPFQQPVDAVKL |
| | | NLPDYYKIIKTPMDMGTIKKRLENNYYWNAQECIQDF |
| | | NTMFTNCYIYNKPGDDIVLMAEALEKLFLQKINELPTE |
| | | E |
| 19 | BRD4 Bromodomain 2 | EQLKCCSGILKEMFAKKHAAYAWPFYKPVDVEALGL |
| | | HDYCDIIKHPMDMSTIKSKLEAREYRDAQEFGADVRL |
| | | MFSNCYKYNPPDHEVVAMARKLQDVFEMRFAKM |

What is claimed:

1. An anti-human CD33 bromo- and extra-terminal domain degrader (BETd) antibody-drug conjugate (ADC) comprising the following structure: 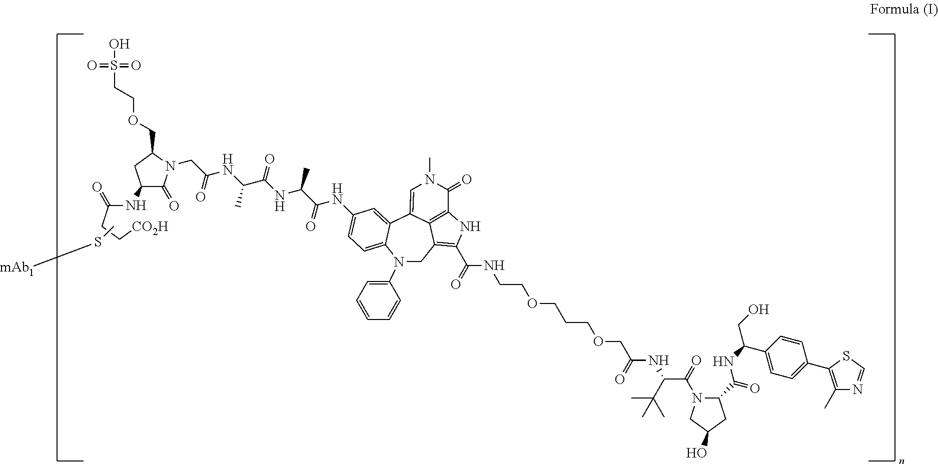
wherein n is an integer from 1 to 10, and wherein mAb1 is an IgG₁ anti-human CD33 antibody comprising:
a heavy chain variable region comprising a CDR-H1, a CDR-H2, and a CDR-H3; and a light chain variable region comprising a CDR-L1, a CDR-L2, and a CDR-L3; and wherein:
CDR-H1 has the amino acid sequence of SEQ ID NO: 1;
CDR-H2 has the amino acid sequence of SEQ ID NO: 2;
CDR-H3 has the amino acid sequence of SEQ ID NO: 3;
CDR-L1 has the amino acid sequence of SEQ ID NO: 4;
CDR-L2 has the amino acid sequence of SEQ ID NO: 5; and
CDR-L3 has the amino acid sequence of SEQ ID NO: 6.

2. The anti-human CD33 BETd ADC of claim 1, wherein mAb1 comprises:
a heavy chain variable region having the amino acid sequence of SEQ ID NO: 7; and
a light chain variable region having the amino acid sequence of SEQ ID NO: 8.

3. The anti-human CD33 BETd ADC of claim 1, wherein mAb1 comprises:
a heavy chain having the amino acid sequence of SEQ ID NO: 9 or SEQ ID NO: 10; and
a light chain having the amino acid sequence of SEQ ID NO: 11.

4. The anti-human CD33 BETd ADC of claim 3, wherein n is about 2.

5. The anti-human CD33 BETd ADC of claim 4, wherein 296C of SEQ ID NO:9 or SEQ ID NO: 10 is the site of conjugation.

6. The anti-human CD33 BETd ADC of claim 3, wherein mAb1 comprises:
a heavy chain having the amino acid sequence of SEQ ID NO: 9; and
a light chain having the amino acid sequence of SEQ ID NO: 11.

7. The anti-human CD33 BETd ADC of claim 6, wherein n is about 2.

8. The anti-human CD33 BETd ADC of claim 7, wherein 296C of SEQ ID NO:9 is the site of conjugation.

9. The anti-human CD33 BETd ADC of claim 3, wherein mAb1 comprises:
a heavy chain having the amino acid sequence of SEQ ID NO: 10; and
a light chain having the amino acid sequence of SEQ ID NO: 11.

10. The anti-human CD33 BETd ADC of claim 9, wherein n is about 2.

11. The anti-human CD33 BETd ADC of claim 10, wherein 296C of SEQ ID NO:9 is the site of conjugation.

12. An anti-human CD33 bromo- and extra-terminal domain degrader (BETd) antibody-drug conjugate (ADC) comprising the following structure: 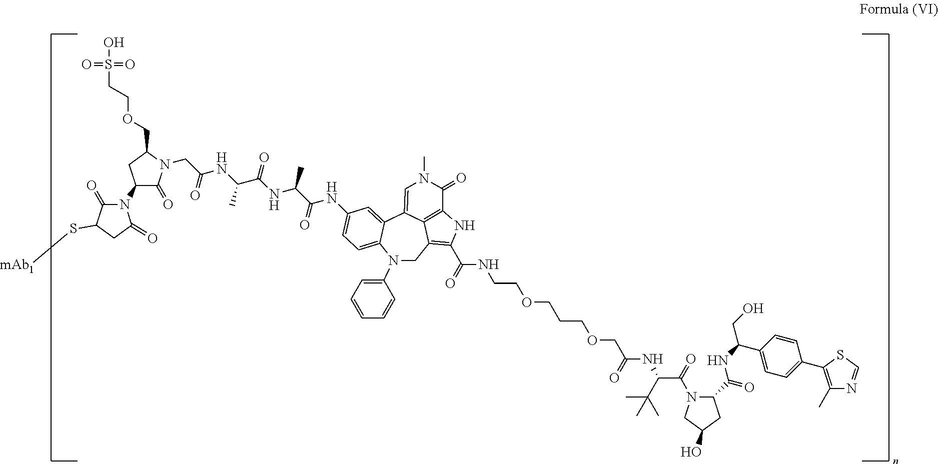
wherein n is about 2, and wherein mAb1 is an IgG₁ anti-human CD33 antibody comprising:
a heavy chain having the amino acid sequence of SEQ ID NO: 9 or SEQ ID NO: 10; and
a light chain having the amino acid sequence of SEQ ID NO: 11.

13. The anti-human CD33 BETd ADC of claim 12, wherein 296C is the site of conjugation.

14. A compound of Formula (III):

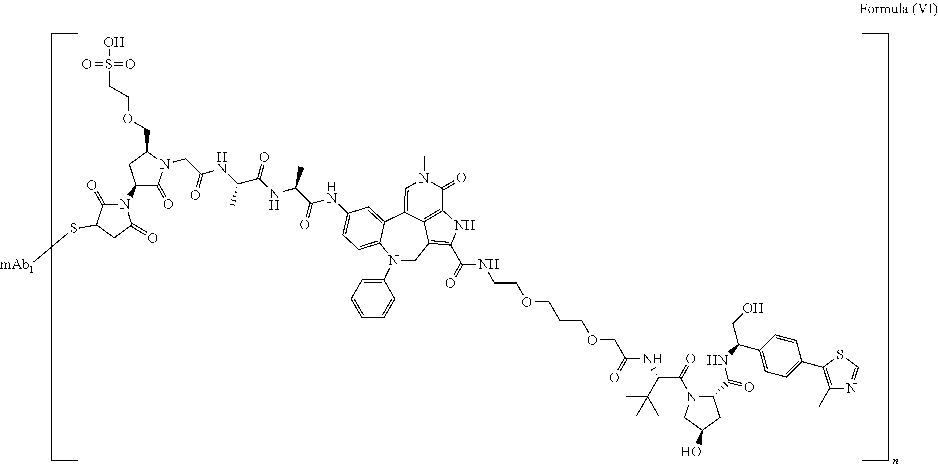

15. A pharmaceutical composition comprising an anti-human CD33 BETd ADC of claims 1-13 and further comprising a pharmaceutically acceptable carrier.

16. A method of treating acute myeloid leukemia, the method comprising administering a therapeutically effective amount of an anti-human CD33 BETd ADC of claims 1-13 to a patient in need thereof.

17. A method of treating acute myeloid leukemia, the method comprising administering a therapeutically effective amount of the pharmaceutical composition of claim 15 to a patient in need thereof.

18. An anti-human CD33 bromo- and extra-terminal domain degrader (BETd) antibody-drug conjugate (ADC) composition comprising the following structure: 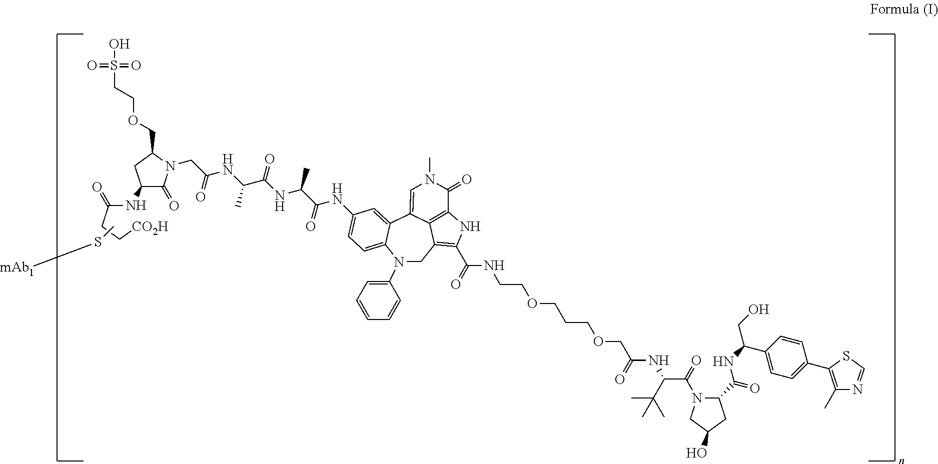
wherein n is about 2, and wherein mAb1 is an IgG₁ anti-human CD33 antibody comprising:
a heavy chain having the amino acid sequence of SEQ ID NO: 9 or SEQ ID NO: 10; and
a light chain having the amino acid sequence of SEQ ID NO: 11.

19. The anti-human CD33 BETd ADC composition of claim 18, wherein 296C is the site of conjugation.

20. A polynucleotide comprising a nucleotide sequence encoding an anti-human CD33 antibody, wherein the antibody comprises a heavy chain variable region comprising a CDR-H1, a CDR-H2, and a CDR-H3; and a light chain variable region comprising a CDR-L1, a CDR-L2, and a CDR-L3; and wherein:
CDR-H1 has the amino acid sequence of SEQ ID NO: 1;
CDR-H2 has the amino acid sequence of SEQ ID NO: 2;
CDR-H3 has the amino acid sequence of SEQ ID NO: 3;
CDR-L1 has the amino acid sequence of SEQ ID NO: 4;
CDR-L2 has the amino acid sequence of SEQ ID NO: 5; and
CDR-L3 has the amino acid sequence of SEQ ID NO: 6.

21. A method for producing an anti-human CD33 bromo- and extra-terminal domain degrader (BETd) antibody-drug conjugate (ADC) comprising the following structure: 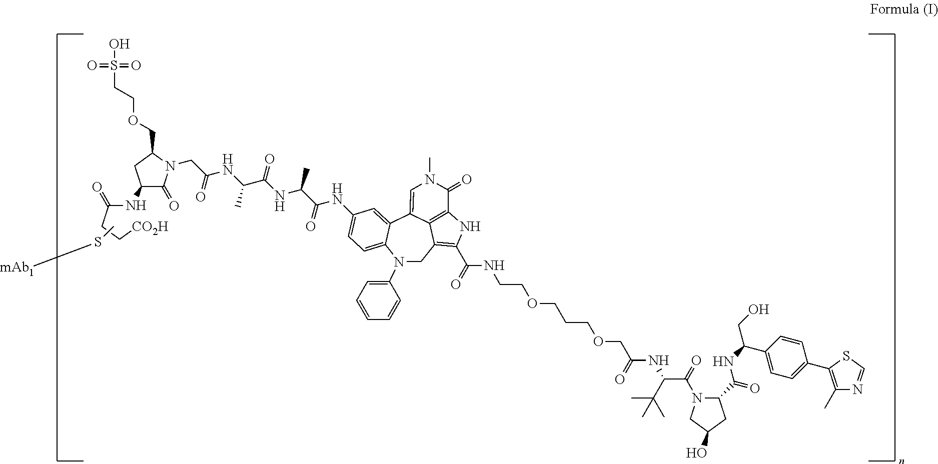
wherein n is an integer from 1 to 10, and wherein mAb1 is an IgG₁ anti-human CD33 antibody comprising:
a heavy chain having the amino acid sequence of SEQ ID NO: 9 or SEQ ID NO: 10; and
a light chain having the amino acid sequence of SEQ ID NO: 11,
wherein the method comprises reducing an antibody with an excess of reducing agent and then conjugating the antibody with an excess of drug-linker.


SEQUENCE LISTING (19 sequences)

SEQ ID NO: 1 - protein, 10 aa, synthetic construct
GFTLSDYAMA

SEQ ID NO: 2 - protein, 17 aa, synthetic construct
TISYDAGRTYYRDAVKG

SEQ ID NO: 3 - protein, 9 aa, synthetic construct
PPGVYYGSY

SEQ ID NO: 4 - protein, 11 aa, synthetic construct
RASQDISNFLN

SEQ ID NO: 5 - protein, 7 aa, synthetic construct
YTSNLQS

SEQ ID NO: 6 - protein, 9 aa, synthetic construct
QQYDESPPT

SEQ ID NO: 7 - protein, 118 aa, synthetic construct
EVQLVESGGGVVQPGRSLRLSCAASGFTLSDYAMAWVRQAPGKGLEWVATISYDAGRTYYRDAVKGRFTISRDNSKNTLYLQMNSLRAEDTAVYYCERPPGVYYGSYWGQGTMVTVSS

SEQ ID NO: 8 - protein, 107 aa, synthetic construct
DIQMTQSPSSLSASVGDRVTITCRASQDISNFLNWYQQKPGKVPKLLIYYTSNLQSGVPSRFSGSGSGTDFTLTISSLQPEDVATYYCQQYDESPPTFGQGTKLEIK

SEQ ID NO: 9 - protein, 448 aa, synthetic construct
EVQLVESGGGVVQPGRSLRLSCAASGFTLSDYAMAWVRQAPGKGLEWVATISYDAGRTYYRDAVKGRFTISRDNSKNTLYLQMNSLRAEDTAVYYCERPPGVYYGSYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQCASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 447 aa, synthetic construct
EVQLVESGGGVVQPGRSLRLSCAASGFTLSDYAMAWVRQAPGKGLEWVATISYDAGRTYYRDAVKGRFTISRDNSKNTLYLQMNSLRAEDTAVYYCERPPGVYYGSYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQCASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 214 aa, synthetic construct
DIQMTQSPSSLSASVGDRVTITCRASQDISNFLNWYQQKPGKVPKLLIYYTSNLQSGVPSRFSGSGSGTDFTLTISSLQPEDVATYYCQQYDESPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - DNA, 1404 bp, synthetic construct
  source 1..1404: mol_type = other DNA
atggagttcggcctcagctggctgtttctggtcgctattctgaaaggcgtgcagtgtgaagtccagctggtggagtccggaggaggagtggtccagcccggaaggtctctgaggctgagctgtgccgctagcggatttacactgagcgactacgccatggcttgggtcagacaagcccccggcaaaggactggagtgggtggctacaatctcctacgacgctggaaggacatactatagggacgccgtgaaaggaaggttcacaatctctagggacaactccaagaacacactgtacctccagatgaactctctgagagccgaggatactgccgtctactactgcgaaaggcctcccggcgtctactatggcagctactggggacaaggcacaatggtgactgtgtccagcgctagcacaaaaggcccatccgtctttcctctggcccctagcagcaaaagcacttccggcggaactgctgctctcggatgtctggtcaaggattacttcccagagccagtgacagtgagctggaacagcggagctctcacttccggcgtgcatactttcccagccgtcctccaatccagcggactgtattctctgtcctccgtggtcactgtgccttccagcagcctcggcacacagacttacatctgcaatgtgaatcacaagccttccaacacaaaggtcgacaagaaggtcgagcctaaaagctgtgacaagactcacacatgccctccttgtccagctccagagctgctgggcggaccatccgtgttcctcttccctcctaagcctaaggacactctgatgatctctaggactccagaggtcacatgtgtcgtggtcgacgtcagccacgaggacccagaggtgaaattcaactggtacgtcgacggcgtggaggtgcacaatgccaaaactaagcctagggaggagcaatgtgccagcacatatagggtcgtgtccgtgctgacagtgctgcaccaagactggctcaacggaaaggagtacaagtgtaaggtctccaataaggccctcccagctccaatcgagaagactatctccaaggccaagggccagcctagggaaccacaagtgtacactctcccaccttctagggaagagatgacaaagaaccaagtgtctctgacttgtctcgtcaagggcttctacccaagcgacattgctgtggagtgggaaagcaacggccagccagaaaacaactacaagacaacaccaccagtgctcgacagcgatggcagcttcttcctctactccaagctgacagtggacaagtctaggtggcagcaaggcaacgtgttcagctgcagcgtcatgcacgaagctctccacaatcactacactcagaagtctctgtctctgtcccccggcaagtga

SEQ ID NO: 13 - DNA, 711 bp, synthetic construct
  source 1..711: mol_type = other DNA
atggacatgcgcgtgcccgcccagctgctgggcctgctgctgctgtggttccccggctcgcgatgcgacatccagatgacccagtctccatcctcactgtctgcatctgtaggagacagagtcaccatcacttgtcgggcgagtcaggatattagcaatttcttaaattggtatcagcagaaaccagggaaagtccctaagctcctgatctattatacatccaacttgcaaagtggggtcccatcaaggttcagcggcagtggatctgggacagatttcactctcaccatcagcagcctgcagcctgaagatgttgcaacttattactgccaacagtatgacgagagccctcccacttttggccaggggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 14 - protein, 460 aa, synthetic construct
EEQVLESGGGLVKPGGSLRLSCAASGFTFSPYSVFWVRQAPGKGLEWVSSINSDSTYKYYADSVKGRFTISRDNAENSIFLQMNSLRAEDTAVYYCARDRSYYAFSSGSLSDYYYGLDVWGQGTTVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 219 aa, synthetic construct
DIVMTQSPLSLSVTPGEPASISCRSSQSLLHTNGYNYLDWYVQKPGQSPQLLIYLASNRASGVPDRFSGSGSGTDFTLKISRVETEDVGVYYCMQALQIPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 451 aa, synthetic construct
EVQLLESGGDLVRPGGSLRLSCAASGFSFSRYGMSWVRQAPGKGLDWVAHISASAGATYYADSVKGRFTISRDNSKNTLFLQMNNLRADDTAIYYCAKGGKQWLIPWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQCASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 214 aa, synthetic construct
DIQMTQSPSSVSASVGDRVTIACRASQDISDRLAWYQQKPGKVPKVLIYGASSLQSGVPSRFSGSGSGTDFTLTINSLQPEDFATYYCQQANSFPLTFGGGTKVEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 112 aa, Homo sapiens
KRQTNQLQYLLRVVLKTLWKHQFAWPFQQPVDAVKLNLPDYYKIIKTPMDMGTIKKRLENNYYWNAQECIQDFNTMFTNCYIYNKPGDDIVLMAEALEKLFLQKINELPTEE

SEQ ID NO: 19 - protein, 106 aa, Homo sapiens
EQLKCCSGILKEMFAKKHAAYAWPFYKPVDVEALGLHDYCDIIKHPMDMSTIKSKLEAREYRDAQEFGADVRLMFSNCYKYNPPDHEVVAMARKLQDVFEMRFAKM